(19) United States
(12) Patent Application Publication
(10) Pub. No.: US 2022/0387600 A1
(43) Pub. Date: Dec. 8, 2022

(54) CONJUGATES AND METHODS FOR TREATING LIVER FIBROSIS

(71) Applicant: GENEVANT SCIENCES GMBH, Basel (CH)

(72) Inventors: James HEYES, Vancouver (CA); Richard J. HOLLAND, Vancouver (CA); Mark WOOD, Moody (CA); Alan D. MARTIN, Vancouver (CA); Christine ESAU, Vancouver (CA); Margrit SCHWARZ, Stow, MA (US); Xin YE, Richmond (CA); Alice Hoy Lam LI, Burnaby (CA); Christopher Justin PASETKA, Langley (CA); David CROWE, Vancouver (CA); Steven TYLER, Vancouver (CA)

(73) Assignee: GENEVANT SCIENCES GMBH, Basel (CH)

(21) Appl. No.: 17/782,901

(22) PCT Filed: Dec. 7, 2020
(86) PCT No.: PCT/US2020/063617

§ 371 Date: Jun. 6, 2022

Related U.S. Application Data
(60) Provisional application No. 62/944,963, filed on Dec. 6, 2019.

(51) Int. Cl. A61K 47/54; C12N 15/113

(52) U.S. Cl. CPC A61K 47/549; C12N 15/113; C12N 2310/351; C12N 2310/14; C12N 2310/321; C12N 2310/322; C12N 2320/32

(57) ABSTRACT

Provided herein are certain nucleic acids (e.g., double stranded siRNA molecules), as well as conjugates that comprise a targeting moiety, a double stranded siRNA, and optional linking groups. Certain embodiments also provide synthetic methods useful for preparing the conjugates. The conjugates are useful to treat certain diseases, such as liver fibrosis, e.g., in the setting of NASH or ASH.

### CROSS-REFERENCE TO RELATED APPLICATION(S)

This patent application claims the benefit of priority of U.S. application Ser. No. 62/944,963, filed Dec. 6, 2019, which application is herein incorporated by reference.

### BACKGROUND

Liver fibrosis is caused by the formation of an abnormally large amount of scar tissue in the liver. Liver fibrosis occurs when the liver attempts to repair and replace damaged cells. Various disorders and drugs can damage the liver and cause fibrosis.

Nonalcoholic fatty liver disease (NAFLD) is a condition in which triglycerides accumulate in the liver. Nonalcoholic steatohepatitis (NASH) is a type of NAFLD. NASH is associated with inflammatory changes and liver cell damage. NASH is a leading cause of liver disease and often progresses to liver fibrosis, cirrhosis and hepatocellular carcinoma (HCC). Non-alcoholic steatohepatitis (NASH) and alcoholic steatohepatitis (ASH) have a similar pathogenesis and histopathology but a different etiology and epidemiology. NASH and ASH are advanced stages of non-alcoholic fatty liver disease (NAFLD) and alcoholic fatty liver disease (AFLD). Alcoholic steatohepatitis (ASH) is a chronic, progressive liver disease characterized by fibrosis of the liver as well as possible necrosis of the liver tissue, brought on by excessive, prolonged alcohol use. Women are more susceptible to the disease because alcohol metabolism is lower in women than in men.

Liver fibrosis is an important underlying cause of liver dysfunction and predicts mortality. Progression to cirrhosis and HCC leads to ultimate liver failure and thus liver transplantation is required. The current US prevalence of NASH-related fibrosis (F2 and later) is about 3.8 million patients. Doctors typically recommend weight loss to treat NAFLD and NASH. While weight loss can reduce fat in the liver, inflammation, and fibrosis, no medicines have been approved to treat NAFLD and NASH. Specifically, no medicines have been approved to treat liver fibrosis. (Clin Liver Dis. 2008 November; 12 (4):733-46, N Engl J Med. 2017 Nov. 23; 377 (21):2063-2072, J Hepatol. 2017 December; 67 (6):1265-127) Accordingly, new therapeutic treatment options are needed for the treatment of liver fibrosis, e.g., in the context of NASH or ASH.

### BRIEF SUMMARY

Accordingly, certain embodiments provide a compound of formula (I):

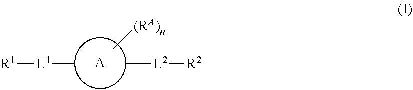

wherein:

R¹ a is targeting ligand;

L¹ is absent or a linking group;

L² is absent or a linking group;

R² is a siRNA molecule selected from a siRNA described herein, e.g., an siRNA selected from any one of siRNA 1-siRNA 119;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂alkyl-OR^{B}, C₁₋₁₀alkyl C₂₋₁₀alkenyl, and C₂₋₁₀alkynyl; wherein the C₁₋₁₀alkyl C₂₋₁₀alkenyl, and C₂₋₁₀alkynyl are optionally substituted with one or more groups independently selected from halo, hydroxy, and C₁₋₃alkoxy;

R^{B} is hydrogen or a protecting group; and

n is 0, 1, 2, 3, 4, 5, 6, 7, 8, 9, or 10;

or a salt thereof.

In certain embodiments, R¹ is —C(H)₍₃₋ₚ₎(L³-saccharide)ₚ;

wherein each L³ is independently a linking group;

p is 1, 2, or 3; and

saccharide is a monosaccharide or disaccharide

or a salt thereof.

In certain embodiments, the saccharide is:

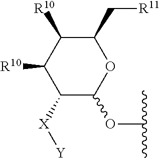

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy;

or a salt thereof.

In certain embodiments, the saccharide is selected from the group consisting of:

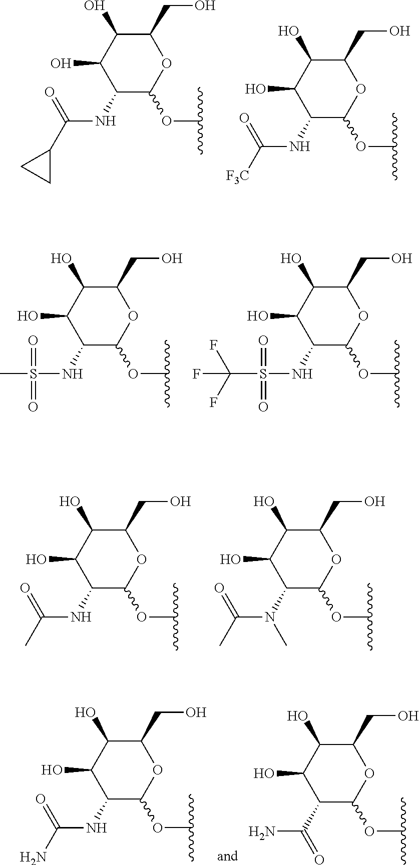

or a salt thereof.

In certain embodiments, the saccharide is:

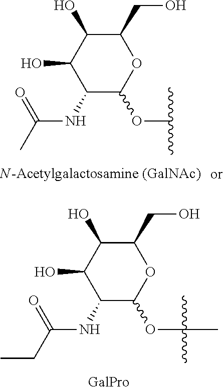 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

or a salt thereof.

In certain embodiments, the compound of formula I is selected from the group consisting of:

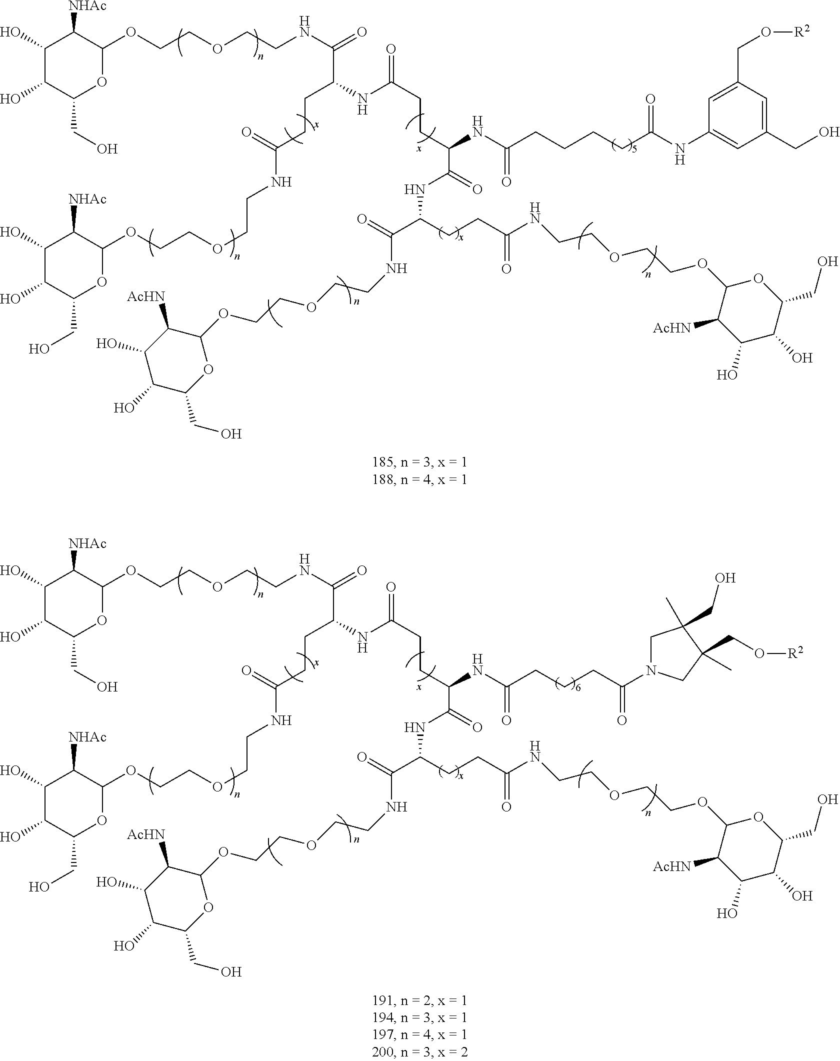

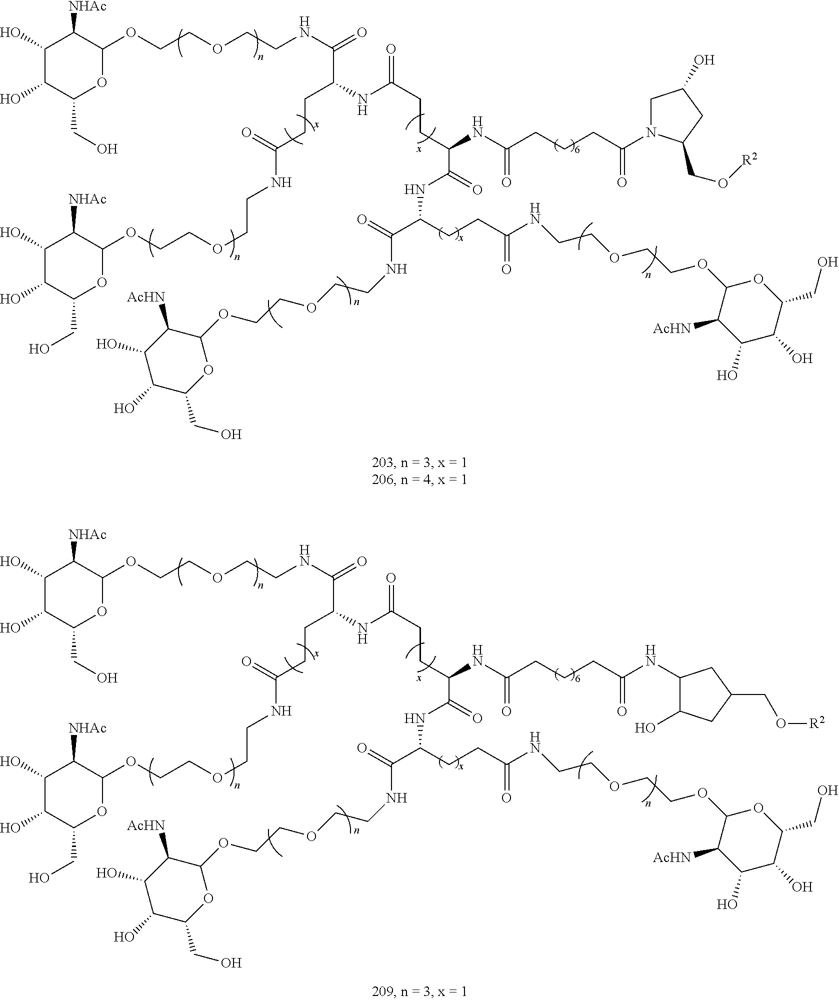

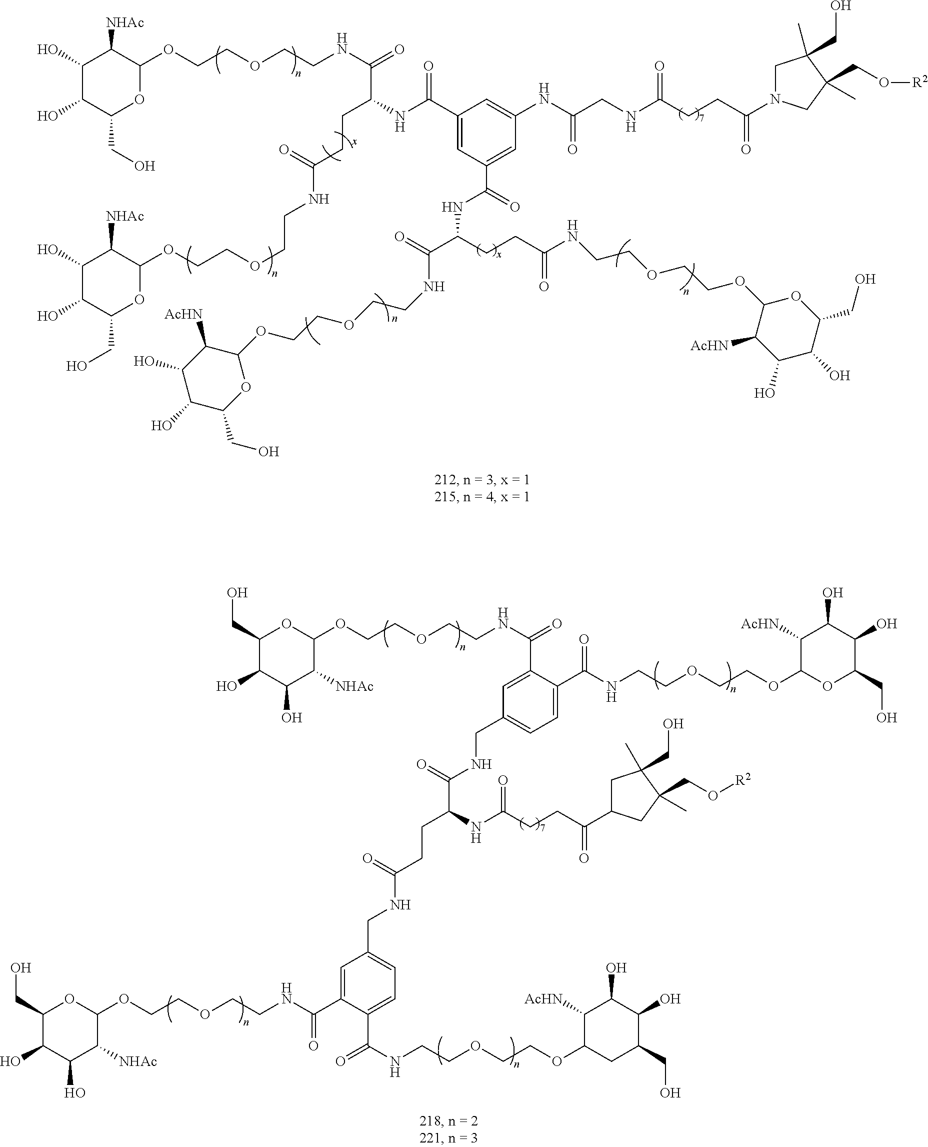

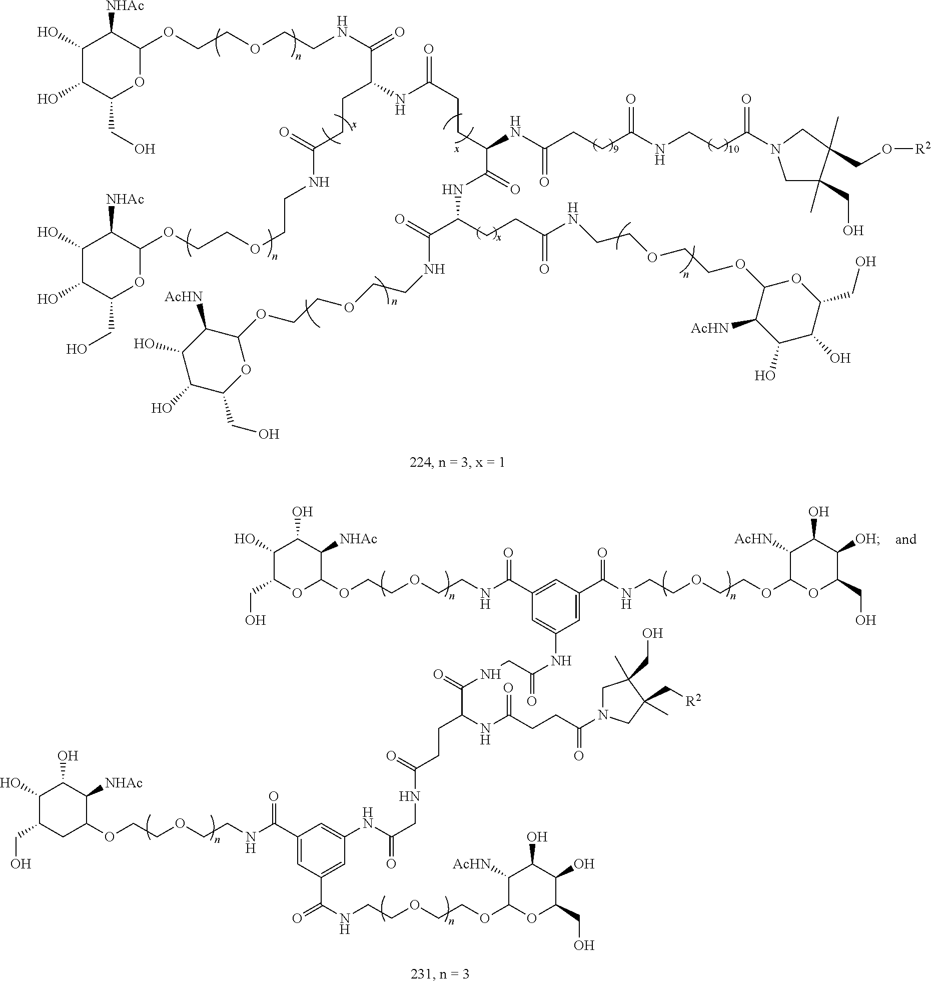

and pharmaceutically acceptable salts thereof.

In certain embodiments, the compound of formula (I) is:

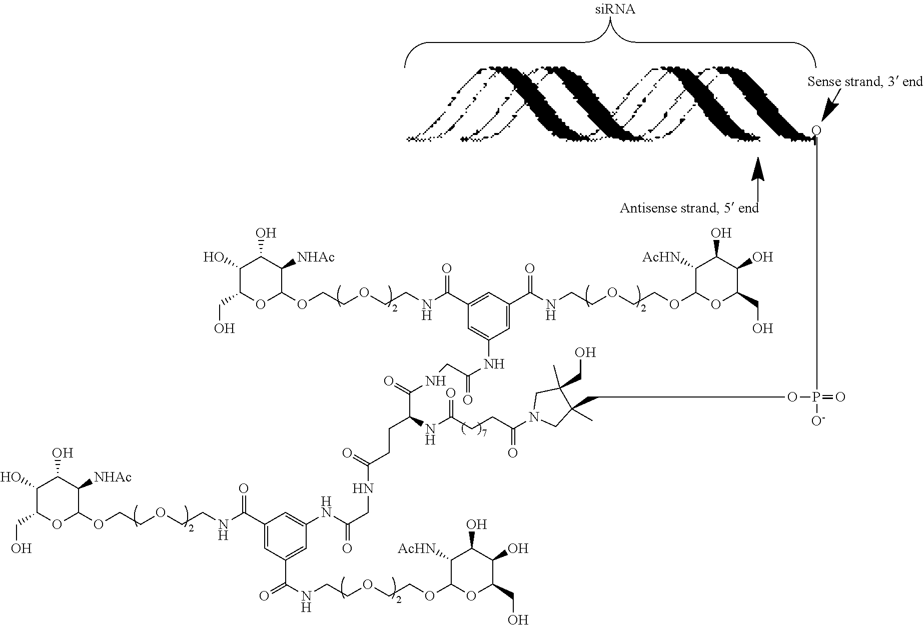

or a pharmaceutically acceptable salt thereof, wherein the siRNA depicted is selected from any one of siRNA 1-siRNA 119.

In certain embodiments, the siRNA sequence comprises chemically modified nucleotides.

In certain embodiments, the siRNA comprises at least one 2′ Ome modification or a 2′F modification.

In certain embodiments, the siRNA comprises at least one 2′ Ome modification and at least one 2′F modification.

In certain embodiments, the siRNA comprises at least one 2′ Ome modification and at least one 2′F modification.

Certain embodiments provide a method of treating liver fibrosis, comprising administering to a patient in need thereof an effective amount of a compound described herein.

Certain embodiments provide a method of treating non-alcoholic steatohepatitis (NASH), comprising administering to a patient in need thereof an effective amount of a compound described herein.

Certain embodiments provide a method of treating liver fibrosis associated with non-alcoholic steatohepatitis (NASH), comprising administering to a patient in need thereof an effective amount of a compound described herein.

Certain embodiments provide a method of treating alcoholic steatohepatitis (ASH), comprising administering to a patient in need thereof an effective amount of a compound described herein.

Certain embodiments provide a method of treating liver fibrosis associated with alcoholic steatohepatitis (ASH), comprising administering to a patient in need thereof an effective amount of a compound described herein.

Certain embodiments provide the use of an effective amount of a compound described herein to treat liver fibrosis.

Certain embodiments provide the use of an effective amount of a compound described herein to treat non-alcoholic steatohepatitis (NASH) or alcoholic steatohepatitis (ASH).

Certain embodiments provide the use of an effective amount of a compound described herein to treat liver fibrosis associated non-alcoholic steatohepatitis (NASH) or alcoholic steatohepatitis (ASH).

In certain embodiments, the compound of formula (I) is administered subcutaneously.

Certain embodiments provide a double stranded siRNA molecule selected from the group consisting of siRNA 1-siRNA 119.

Certain embodiments provide a composition comprising a double stranded siRNA molecule of claim 21.

Certain embodiments provide an invention as described herein.

In certain embodiments, provided herein are nucleic acid molecules (e.g., therapeutic double stranded siRNA molecules), as well as conjugates, compositions and methods that can be used to deliver such nucleic acids. These are useful for treating liver fibrosis, e.g., NASH-or ASH-related liver fibrosis.

Accordingly, one aspect provides a double stranded siRNA molecule selected from the group consisting of siRNA 1-siRNA 119, and to individual sense and anti sense strands thereof.

Another aspect provides GalNAc conjugates that comprise one of the siRNAs described herein, which conjugates are not limited to conjugates that comprise the ligand-linkers disclosed herein. For example, an aspect provides a GalNAc conjugate of Formula X:

A-B-C (X)

wherein A is a targeting ligand;
B is an optional linker; and
C is an siRNA molecule described herein.

Additional conjuagtes useful with the siRNA molecules described herein are described in WO 2017/177326 (PCT/CA2017/050447) and in WO 2018/191278 (PCT/US2018/026918), the disclosures of which are each incorporated by reference.

Provided herein are also synthetic intermediates and methods disclosed herein that are useful to prepare compounds of formula I.

Other objects, features, and advantages will be apparent to one of skill in the art from the following detailed description and figures.

### BRIEF DESCRIPTION OF THE FIGURES

FIG. 1. FIG. 1 provides nucleic acid sequences (e.g., siRNA sequences) of certain embodiments of the invention. FIG. 1 depicts both unmodified sense and unmodified antisense sequences and modified sense and modified antisense sequences. Certain embodiments of the invention are directed to the described modified sequences, siRNA molecules comprising the same, and conjugates comprising the same. Certain embodiments of the invention are directed to modified nucleic acid sequences that comprise the sequence of the unmodified sense and unmodified antisense sequences, which modified sequence comprises at least one chemical modification (e.g., at least one 2′ Ome modification and/or at least one 2′F modification), siRNA molecules comprising the same, and conjugates comprising the same. For the HSD17B13-targeting GalNAc-siRNA conjugates listed, sequence annotation: lower case=2′ Ome modification; upper case+f=2′F modification; phosphorothiorate bond=s; upper case alone=ribonucleotide).

FIG. 2. FIG. 2 provides in vitro activity (dual dose 10 and 0.1 nM) and target sites of siRNA conjugates of certain embodiments of the invention. GalNAc-siRNA conjugates targeting HSD17B13 were incubated with primary human hepatocytes at 0.1 or 10 nM final concentration for 48 h. The intracellular HSD17B13 mRNA levels were quantified with bDNA Quantigene assay. Certain embodiments of the invention are directed to siRNA molecules, e.g., chemically-modified siRNA molecules, and conjugates thereof, that target one of the sites described in FIG. 2.

FIG. 3. FIG. 3 depicts in vitro activity (IC50 in primary human hepatocytes) of certain siRNA conjugates of the invention. GalNAc-siRNA conjugates targeting HSD17B13 were incubated with primary human hepatocytes at 10 different doses for 48 h. The intracellular HSD17B13 mRNA levels were quantified with bDNA Quantigene assay. IC50 values were determined using 4-parameter sigmoidal curve fit.

FIG. 4: FIG. 4 depicts in vivo activity in non-human primates (NHP) of certain siRNA conjugates of the invention (see Example 1). Single doses of GalNAc-siRNA conjugates were injected into male cynomolgus monkeys subcutaneously. At 14 days post injection, the liver from each animal was collected and the hepatic HSD17B13 mRNA level was determined by RT-qPCR and normalized to the average of 3 endogenous control mRNA levels (GAPDH, Arf1 and Eif1). The ratio of HSD17B13/endogenous control was further normalized to that observed in livers of saline treated animals. Results for GalNAc-siRNA conjugates of siRNA 28, 86 and 59, from left to right, are provided.

FIG. 5. FIG. 5 depicts overall mortality stratified on fibrosis stage compared to matched controls. Interestingly, fibrosis stage but not NASH predicts mortality.

FIG. 6. FIG. 6 depicts HSD17B13 as a unique target as a 300 amino acid protein predominantly expressed in liver. While not to be bound by such, a mechanistic hypothesis of importance is related to generation of cytotoxic lipids, and overexpression correlates with NAFLD in humans. Splice variant giving rise to low-abundance truncated hepatic protein associated with reduced NASH risk, fibrosis and HCC, even in people with genetic NASH predisposition. PNPLA3 SNP is reliable genetic marker for patient segmentation leading to increased clinical probability of success. HSD17B13 may be involved in generation of lipid droplet associated lipotoxic lipids. Lipotoxic lipids are one of two pathophysiologies leading to hepatic fibrosis. Silencing HSD17B13 should reduce lipotox and reduce or halt fibrosis. Accordingly, certain embodiments of the invention are directed to siRNA molecules, e.g., chemically-modified siRNA molecules, and conjugates thereof, that target HSD17B13.

FIG. 7. FIG. 7 depicts comparative results with other comparison compounds (see WO 2019/183164). There was no statistical significance among the different leads, dosed at 3 mg/kg single dose, using the same Thermofisher Taqman qPCR assay. (ALNY−n=3, gender not specified, Day 21, normalized to GAPDH only; as compared to a representative conjugate of the invention (GalNAc-siRNA conjugates of siRNA 28)−n=4, male, Day 14, normalized to 3 endogenous genes to reduce analysis bias.

FIG. 8. FIG. 8 provides a summary of results related to certain conjugates of the invention, from top to bottom, results for GalNAc-siRNA conjugates of siRNAs 37, 28, 86, 59, 40 and 85 are provided.

FIG. 9. FIG. 9A depicts rodent safety screening results for certain conjugates of the invention using multiple doses. Results for saline and GalNAc-siRNA conjugates of siRNA 37, 28 and 86, from left to right for each, are provided in the top panels. Results for saline and GalNAc-siRNA conjugates of siRNA 59, 40 and 85, from left to right for each, are provided in the bottom panels. FIG. 9B depicts rodent safety screening results for certain conjugates of the invention using a single dose. Results for saline and GalNAc-siRNA conjugates of siRNA 37, 28 and 86, from left to right for each, are provided in the top panels. Results for saline and GalNAc-siRNA conjugates of siRNA 59, 40 and 85, from left to right for each, are provided in the bottom panels. Further, while not depicted, results of safety profiles performed in non-human primates found no adverse findings with a dosage of 3 mg/kg.

FIG. 10. FIG. 10A depicts alignments of certain siRNA of the invention with HSD17B13 variants (GalNAc-siRNA conjugates of siRNAs 28, 86 and 59, from left to right). As depicted in FIG. 10B, variants A and D are the dominant transcripts in human.

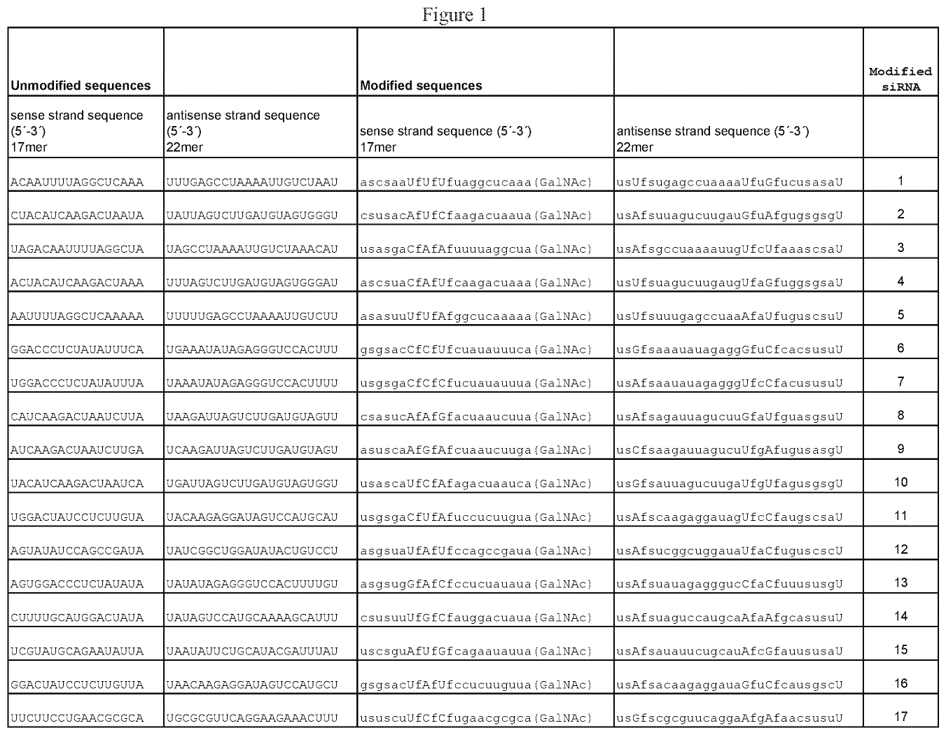

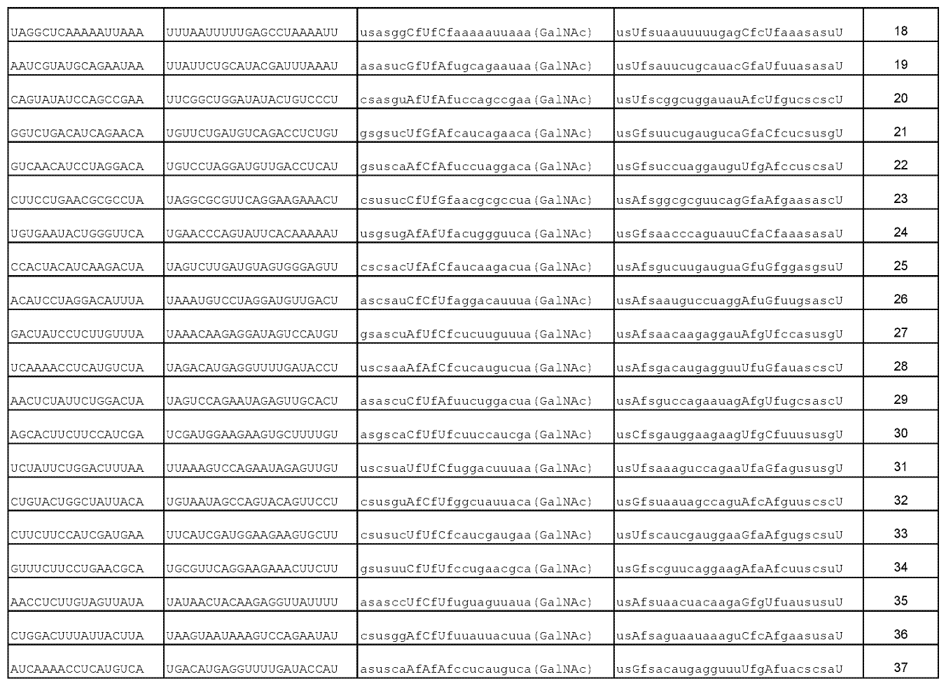

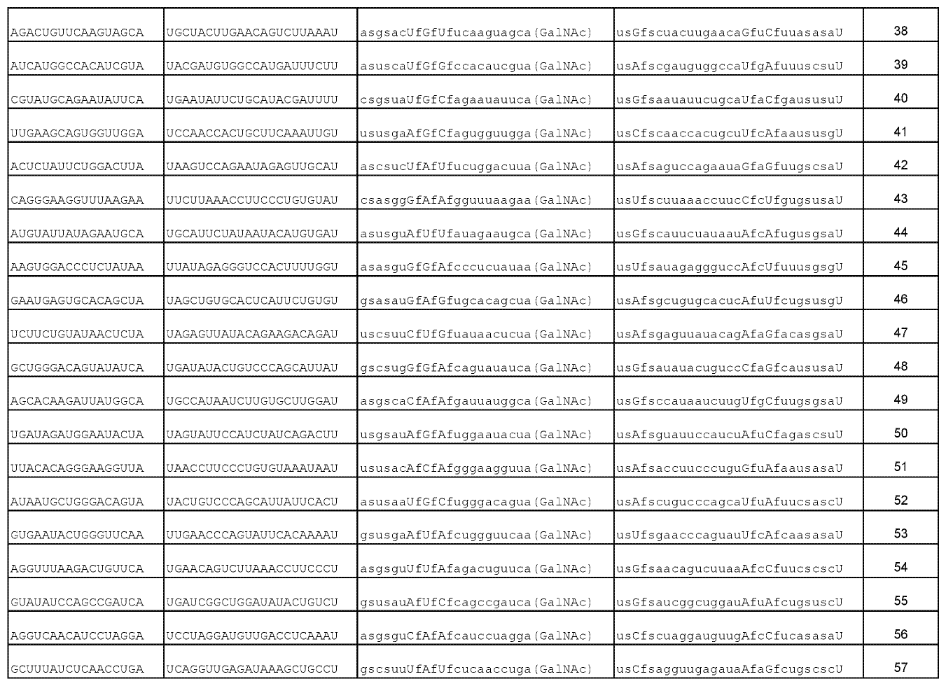

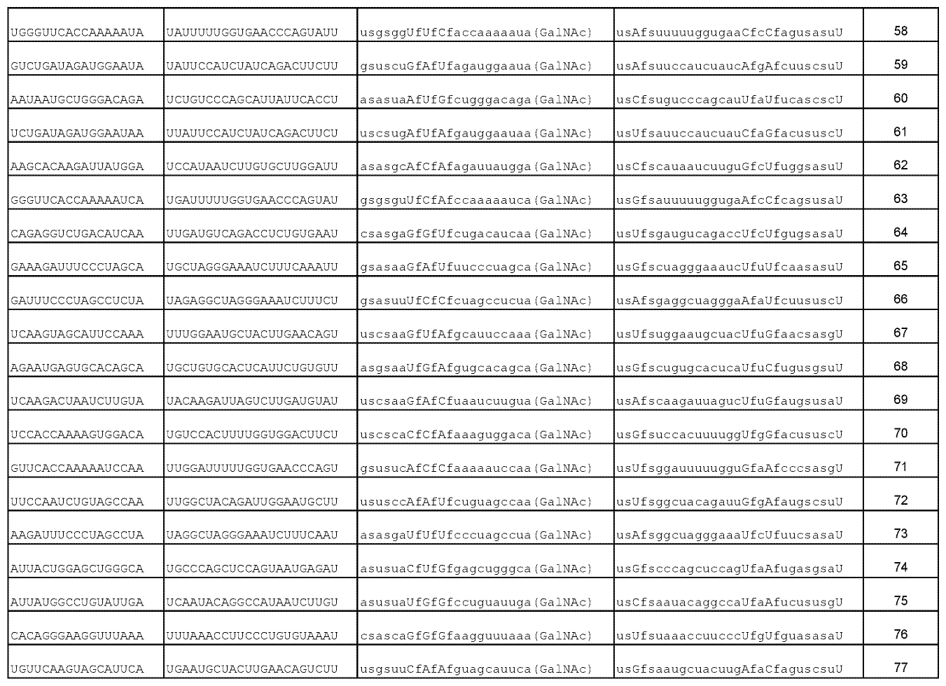

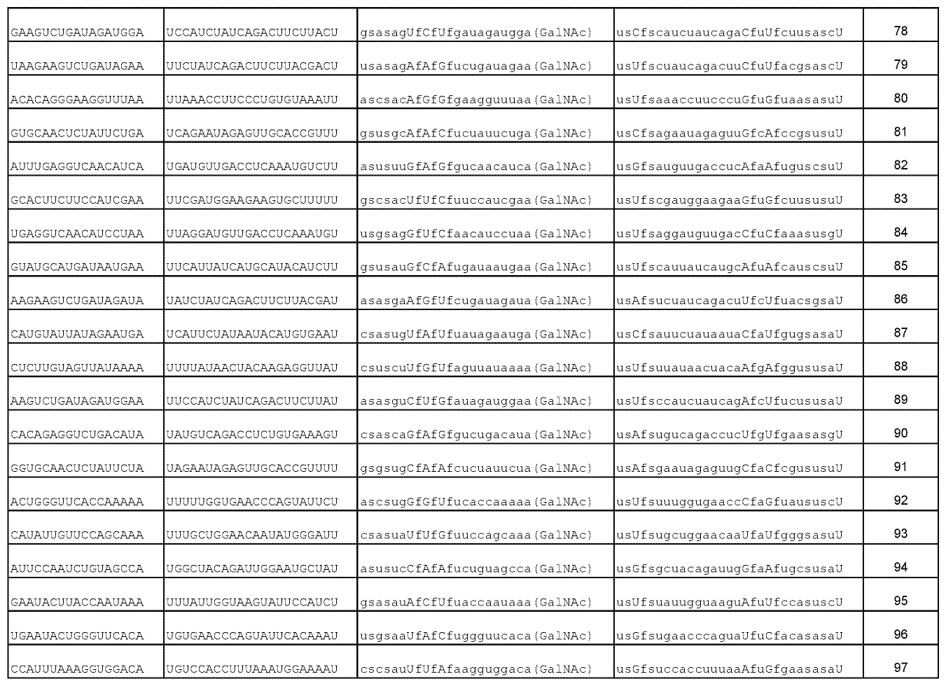

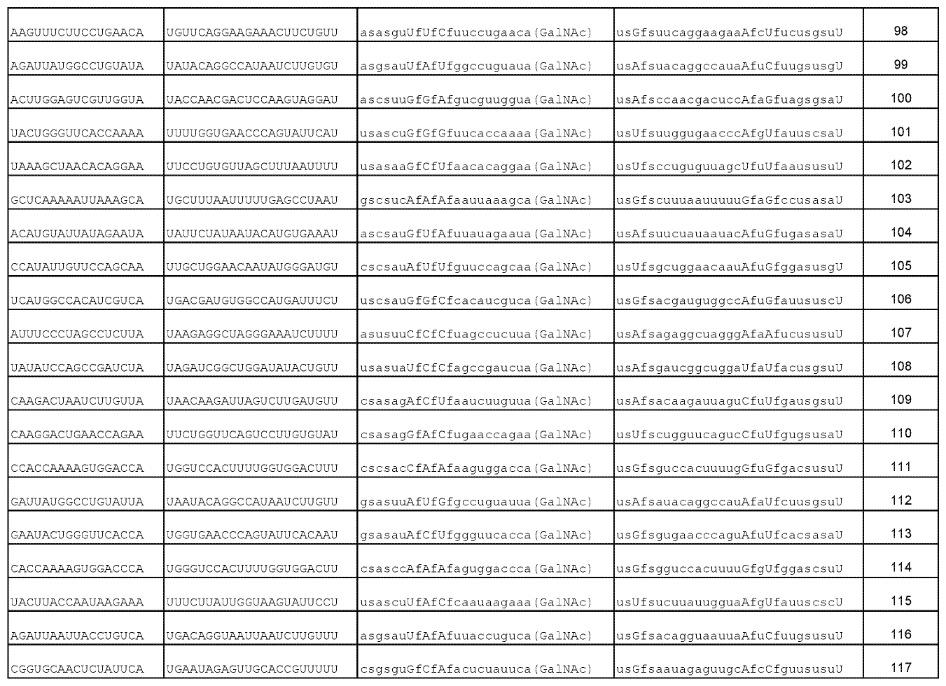

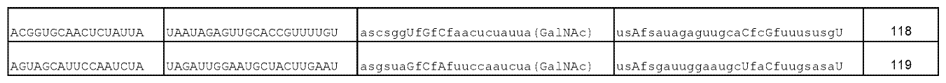

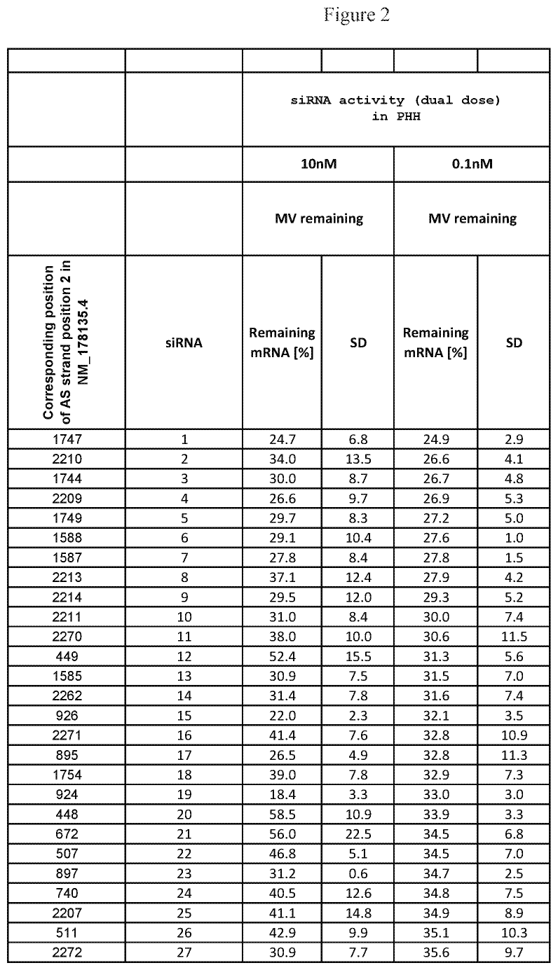

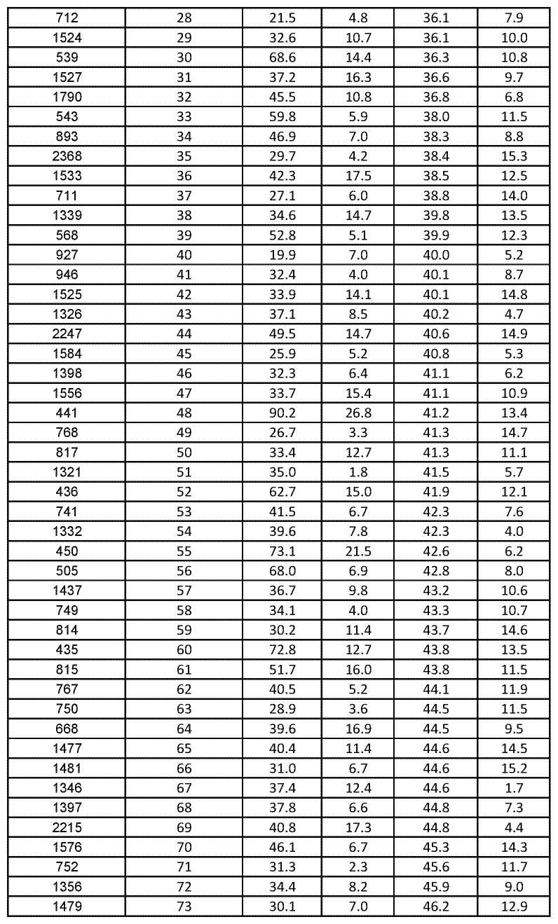

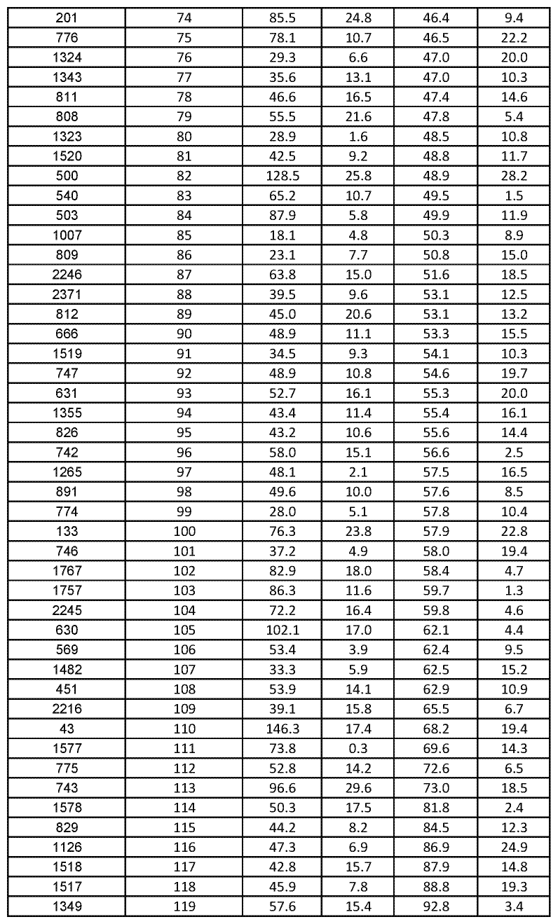

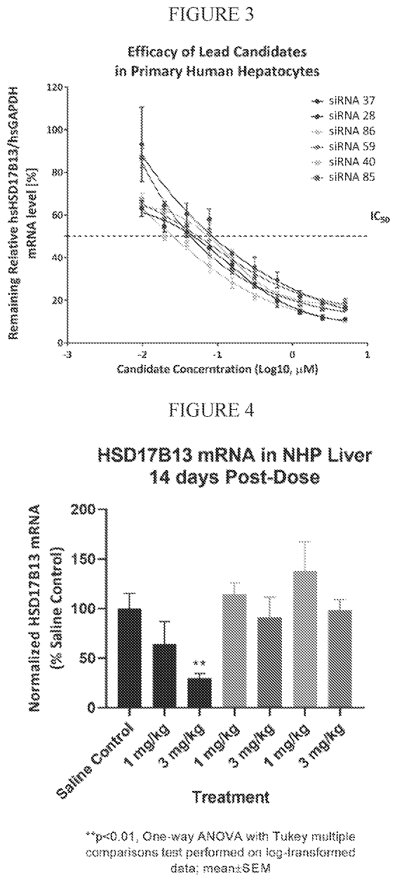

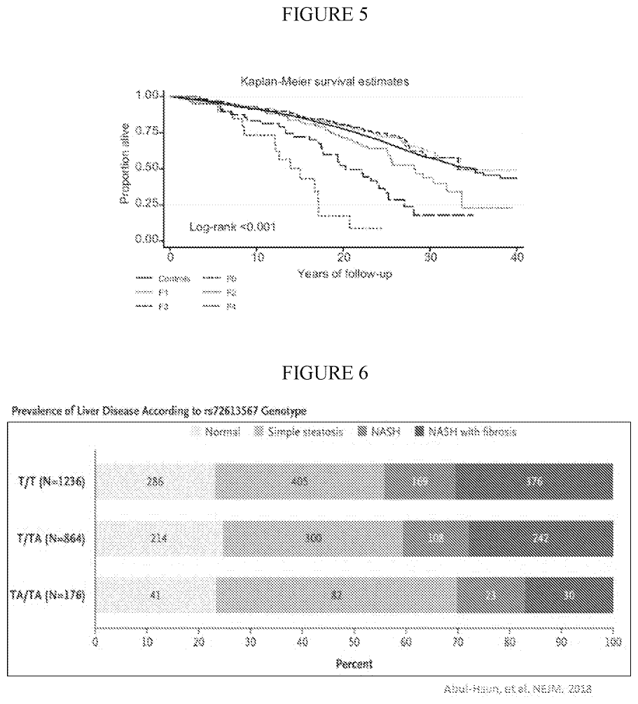

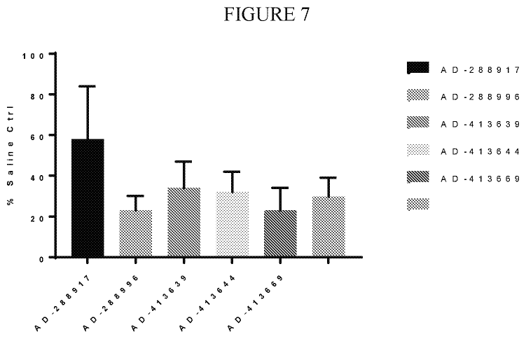

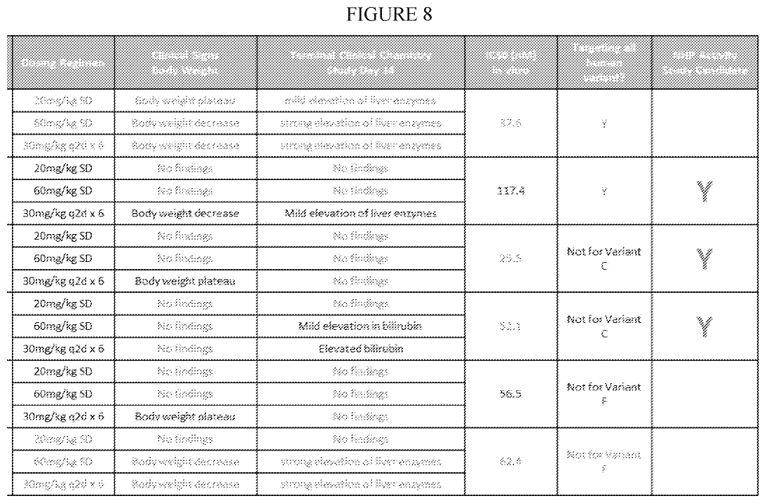

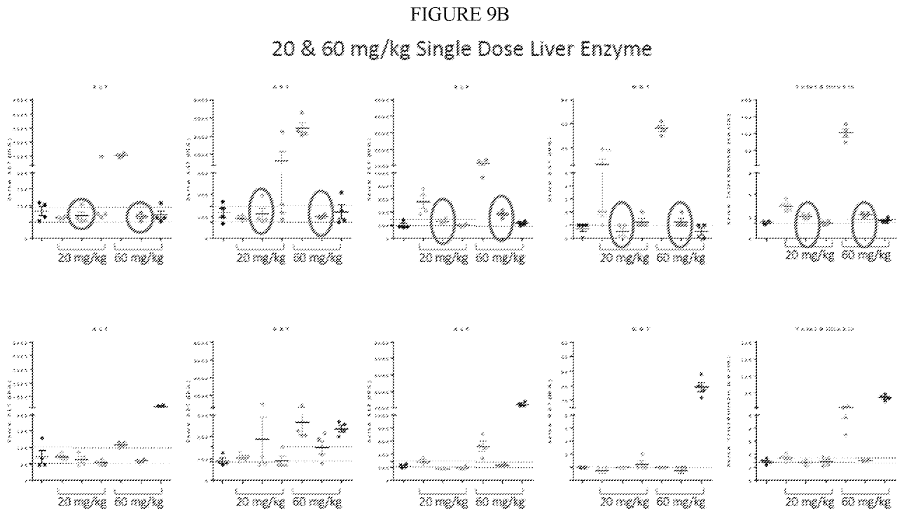

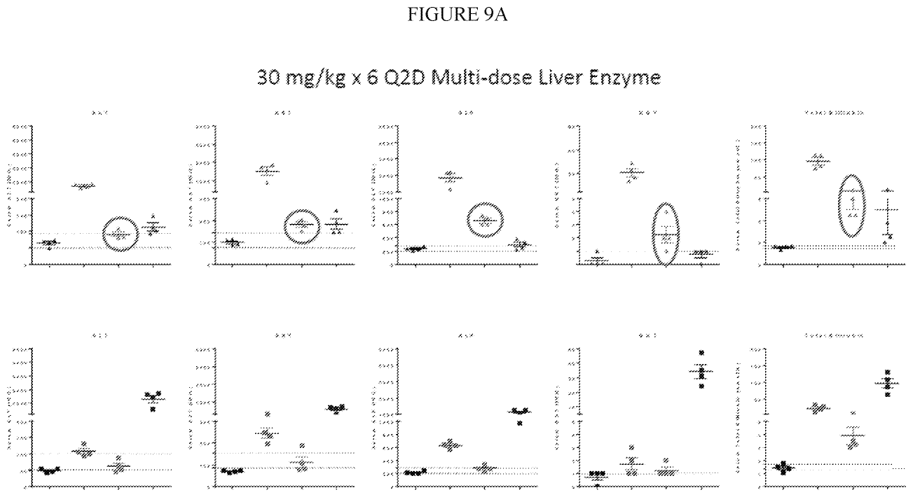

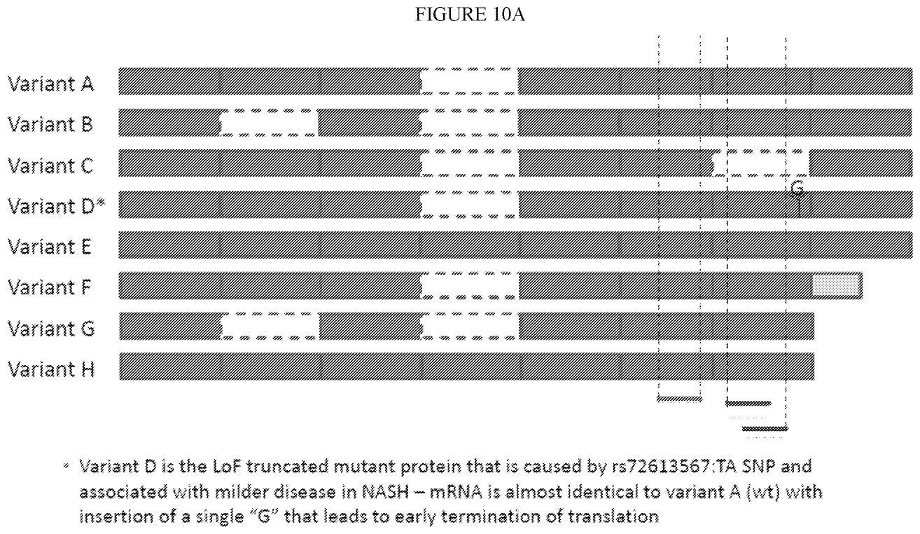

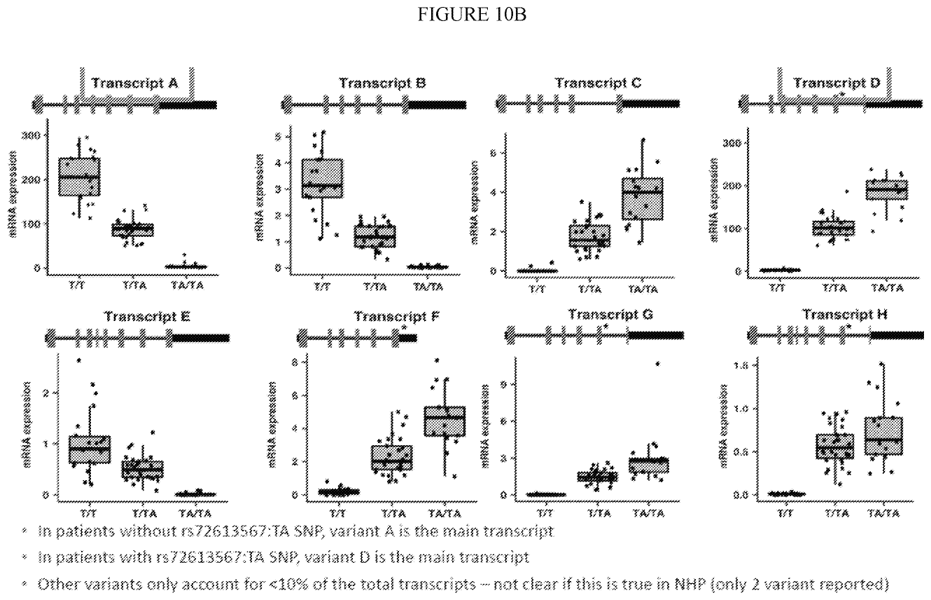

In the application, including Figures, Examples and Schemes, it is to be understood that an oligonucleotide can be, e.g., a double stranded siRNA molecule as described in FIG. 1.

### DETAILED DESCRIPTION

HSD17B13 is predominantly expressed in the liver. Its overexpression correlates with NAFLD in humans. Human genetic data showed that splice variants giving rise to low-abundance truncated hepatic protein were associated with reduced NASH risk, fibrosis and HCC, indicating that loss of function in HSD17B13 may protect liver from NASH-related fibrosis. The RNAi strategy described herein allows more targeted inhibition of target genes as compared to other methods, and in certain embodiments, at a later stage of disease. These finding provide new means to treat liver fibrosis, e.g., NASH or ASH with liver fibrosis.

As described herein, a single dose of a conjugate of the invention can efficiently reduce HSD17B13 expression in NHP. Further, comparative data demonstrated that a conjugate of the invention demonstrates comparable in vivo activity to comparator conjugates from other companies in cynomolgus monkey experiments.

Accordingly, provided herein is a compound of formula (I):

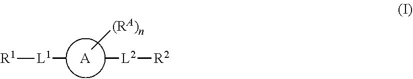

wherein:

R¹ a is targeting ligand;

L¹ is absent or a linking group;

L² is absent or a linking group;

R² is a siRNA molecule selected from any one of the siRNA disclosed herein, e.g., selected from siRNA 1-siRNA 119;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂alkyl-OR^{B}, C₁₋₁₀alkyl C₂₋₁₀alkenyl, and C₂₋₁₀alkynyl; wherein the C₁₋₁₀alkyl C₂₋₁₀alkenyl, and C₂₋₁₀alkynyl are optionally substituted with one or more groups independently selected from halo, hydroxy, and C₁₋₃alkoxy;

R^{B} is hydrogen or a protecting group; and

n is 0, 1, 2, 3, 4, 5, 6, 7, 8, 9, or 10;

or a salt thereof.

In certain embodiments, R¹ is —C(H)₍₃₋ₚ₎(L³-saccharide)ₚ;

wherein each L³ is independently a linking group;

p is 1, 2, or 3; and

saccharide is a monosaccharide or disaccharide

or a salt thereof.

In certain embodiments, the saccharide is:

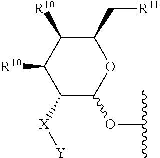

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy;

or a salt thereof.

In certain embodiments, the saccharide is selected from the group consisting of:

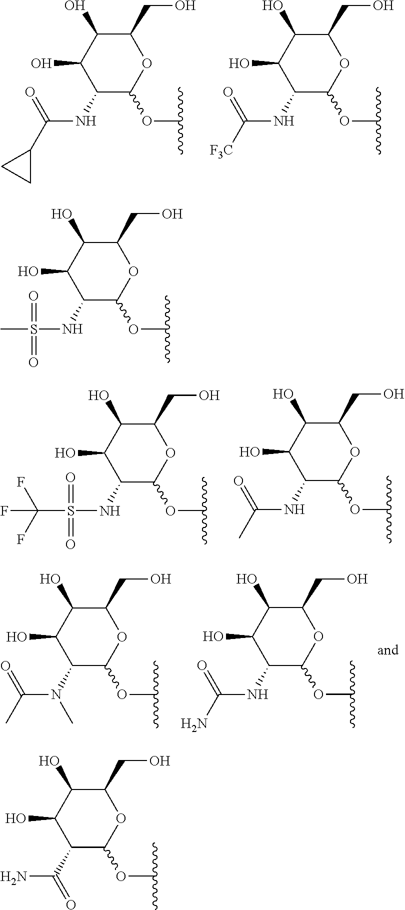

or a salt thereof.

In certain embodiments, the saccharide is:

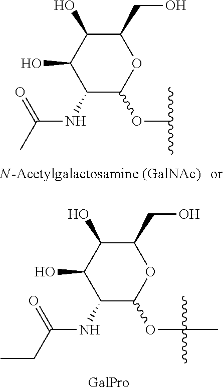 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

or a salt thereof.

In certain embodiments, the compound of formula I is selected from the group consisting of:

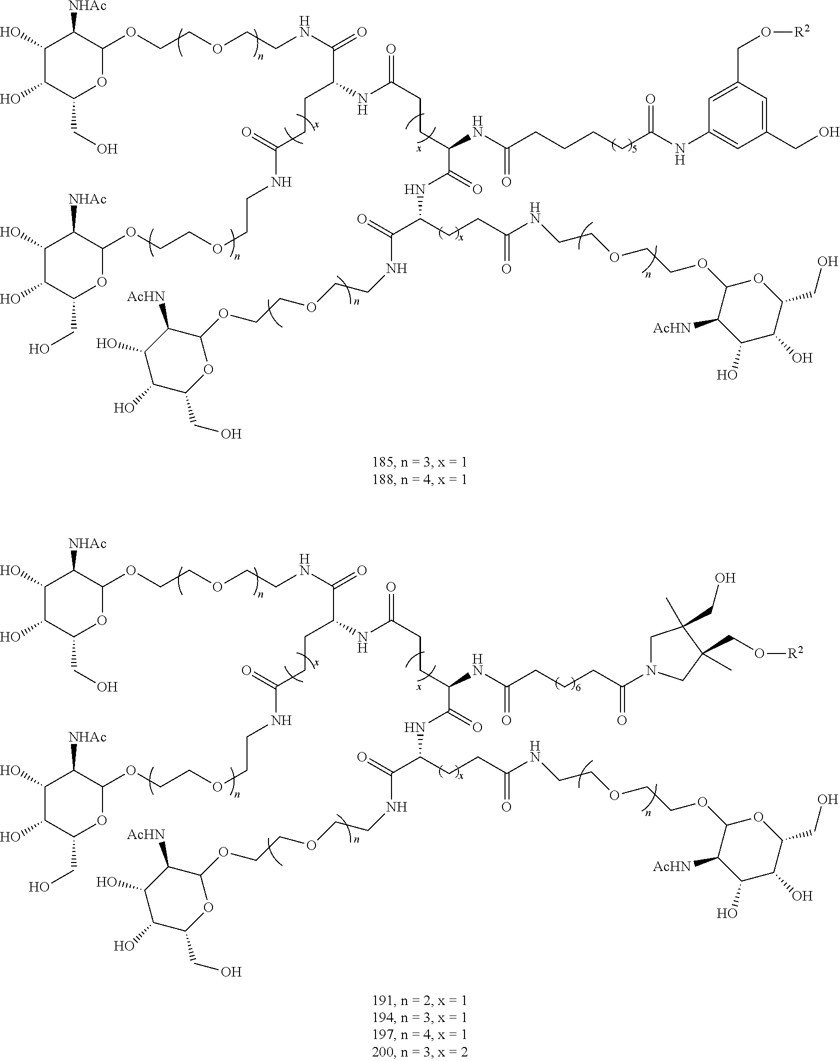

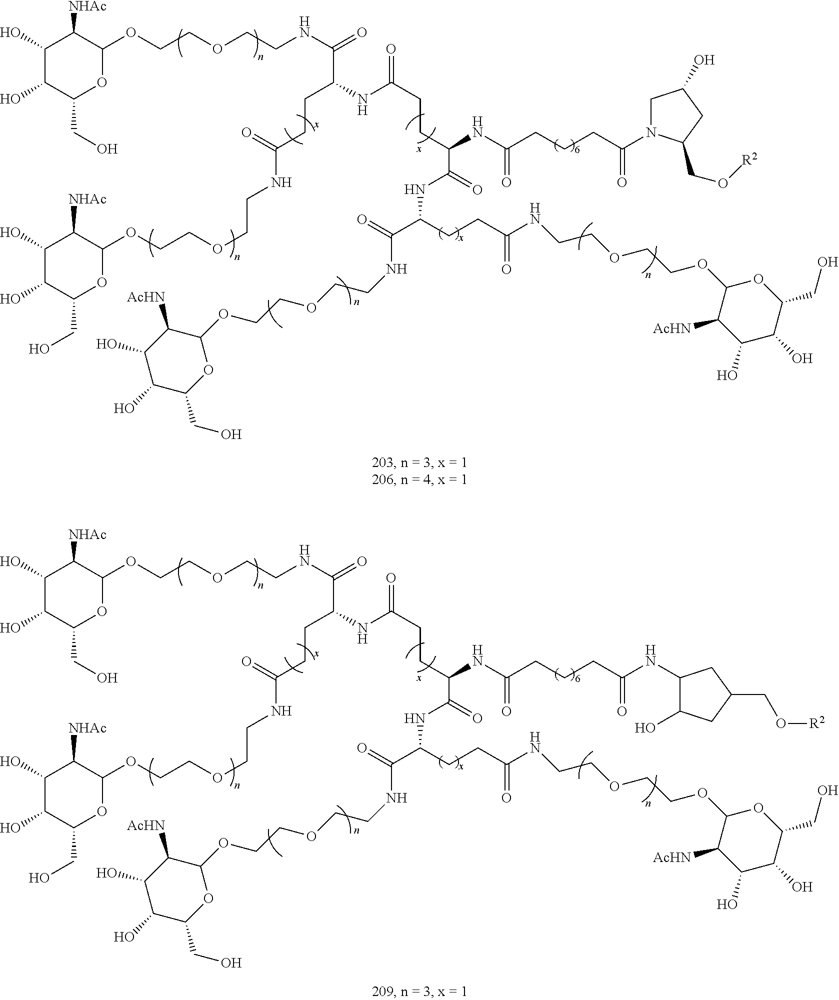

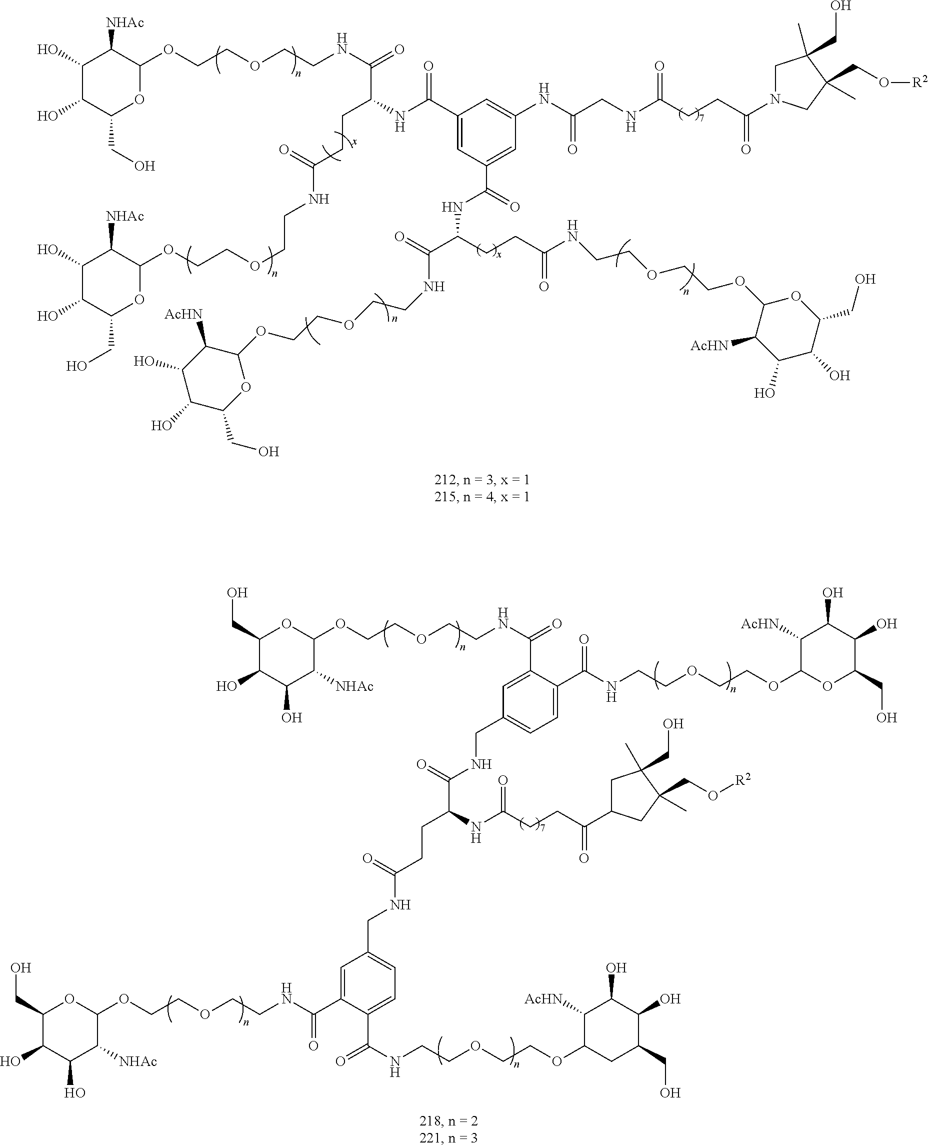

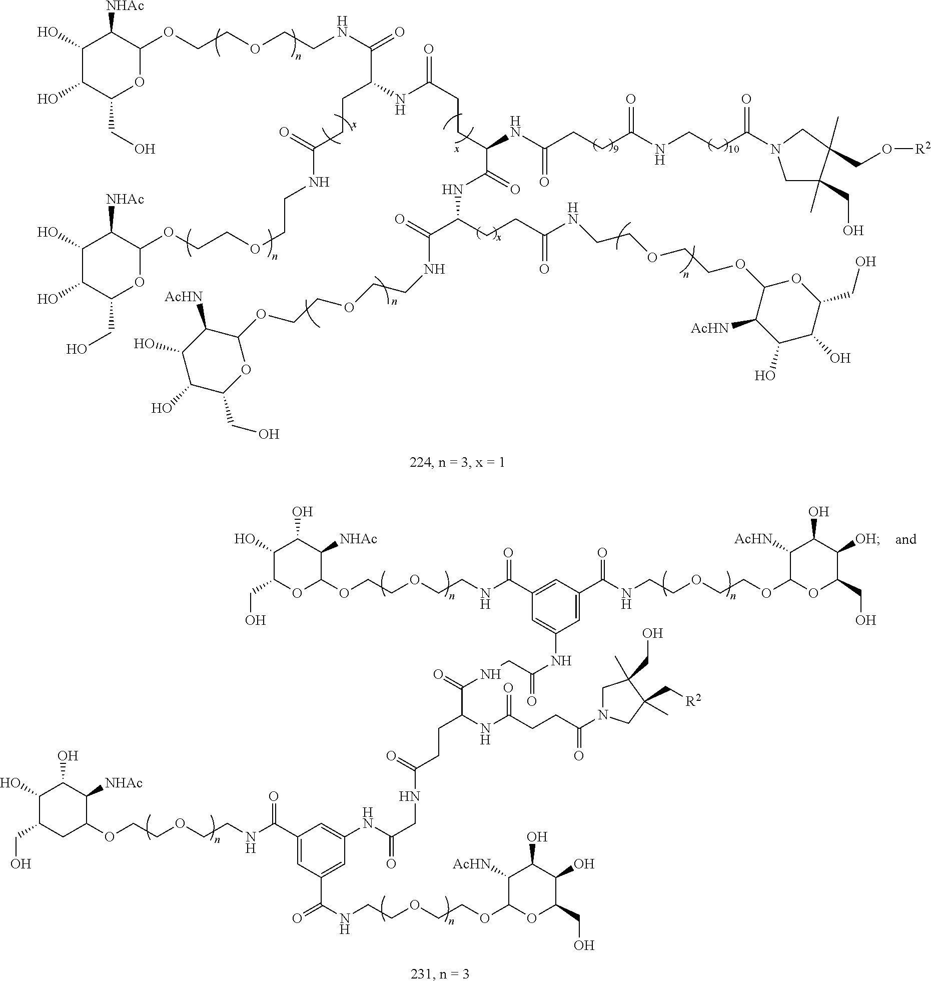

and pharmaceutically acceptable salts thereof.

In certain embodiments, the compound of formula (I) is:

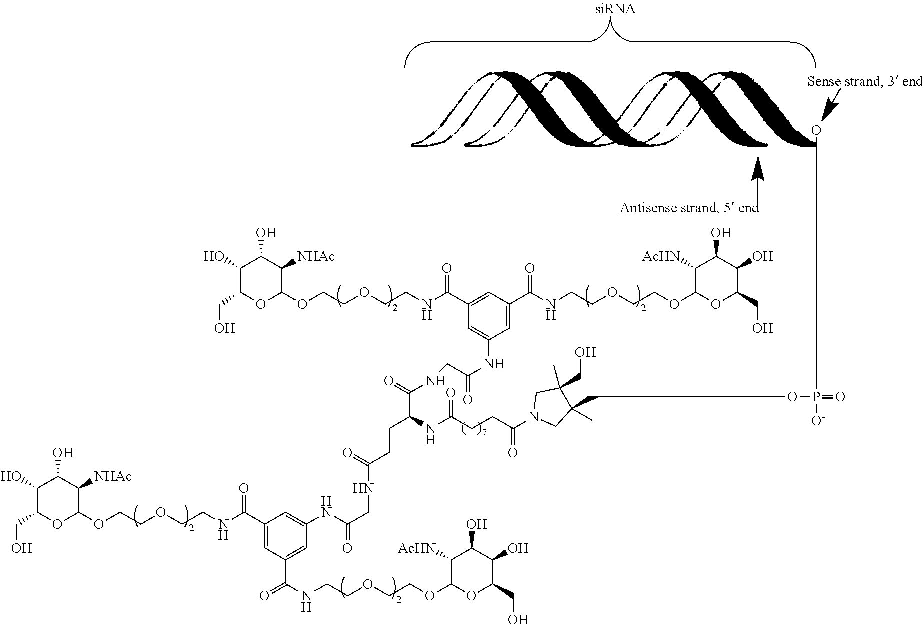

or a pharmaceutically acceptable salt thereof, wherein the siRNA depicted is selected from any one of siRNA 1-siRNA 119.

Certain embodiments provide a method for treating liver fibrosis, e.g., in the setting of NASH or ASH, comprising administering to a patient in need thereof an effective amount of a compound as described herein.

In certain embodiments, the compound of formula (I) is administered subcutaneously.

Certain embodiments provide a double stranded siRNA molecule selected from the group consisting of siRNA 1-siRNA 119.

As used herein, the following terms have the meanings ascribed to them unless specified otherwise.

The siRNA molecules and conjuagtes described herein can be used, in certain embodiments, in combination with surgical treatment, radiation treatment (e.g., conventional radioation therapy, proton beam therapy or stereotaxic radiosurgery), and/or other medications.

The term “conjugate” as used herein includes compounds of formula (I) that comprise an oligonucleotide (e.g., an siRNA molecule) linked to a targeting ligand. Thus, the terms compound and conjugate may be used herein interchangeably.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and in certain embodiments is about 20-24, 21-22, or 21-23 (duplex) nucleotides in length. siRNA duplexes may comprise 3′ overhangs, e.g., of about 1 to about 5 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand.

In certain embodiments, the 5′ and/or 3′ overhang on one or both strands of the siRNA comprises 1-5 (e.g., 1, 2, 3, 4 or 5) modified and/or unmodified deoxythymidine (t or dT) nucleotides, 1-5 (e.g., 1, 2, 3, 4 or 5) modified (e.g., 2′OMe) and/or unmodified uridine (U) ribonucleotides, and/or 1-5 (e.g., 1, 2, 3, 4 or 5) modified (e.g., 2′OMe) and/or unmodified ribonucleotides or deoxyribonucleotides having complementarity to the target sequence (e.g., 3′ overhang in the antisense strand) or the complementary strand thereof (e.g., 3′ overhang in the sense strand).

In certain embodiments, siRNA are chemically synthesized. siRNA can also be generated by cleavage of longer dsRNA with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10 (1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). In certain embodiments, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA to silence, reduce, or inhibit expression of a target gene. To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “synthetic activating group” refers to a group that can be attached to an atom to activate that atom to allow it to form a covalent bond with another reactive group. It is understood that the nature of the synthetic activating group may depend on the atom that it is activating. For example, when the synthetic activating group is attached to an oxygen atom, it will activate that oxygen atom to form a bond (e.g. an ester, carbamate, or ether bond) with another reactive group. Such synthetic activating groups are known. Examples of synthetic activating groups that can be attached to an oxygen atom include, but are not limited to, acetate, succinate, triflate, and mesylate. When the synthetic activating group is attached to an oxygen atom of a carboxylic acid, the synthetic activating group can be a group that is derivable from a known coupling reagent (e.g. a known amide coupling reagent). Such coupling reagents are known. Examples of such coupling reagents include, but are not limited to, N,N′-Dicyclohexylcarbodimide (DCC), hydroxybenzotriazole (HOBt), N-(3-Dimethylaminopropyl)-N′-ethylcarbonate (EDC), (Benzotriazol yloxy)tris(dimethylamino)phosphonium hexafluorophosphate (BOP), benzotriazol-1-yl-oxytripyrrolidinophosphonium hexafluorophosphate (PyBOP) or O-benzotriazol-1-yl-N,N,N′,N′-tetramethyluronium hexafluorophosphate (HBTU).

An “effective amount” or “therapeutically effective amount” of a therapeutic nucleic acid such as siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art. In certain embodiments, a therapeutically effective amount is demonstrated by an improvement in liver fibrosis, demonstrated, e.g., by improvement in fibrosis biomarkers.

In certain embodiments, a therapeutically effective amount is demonstrated by a combination of improvement in NASH/NAFLD activity scores supported by improvement in fibrosis biomarkers.

In certain embodiments, a therapeutically effective amount is demonstrated by a combination of improvement in ASH supported by improvement in fibrosis biomarkers.

In certain embodiments, a therapeutically effective amount is demonstrated by an improvement in markers for liver inflammation and liver function, improved health-related quality of life, and/or by improved liver function tests (AST, ALT, GGT, ALP).

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Accordingly, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

As used herein, the term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkynyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane (including straight and branched alkanes), as exemplified by —CH₂CH₂CH₂CH₂— and —CH(CH₃)CH₂CH₂—.

The term “cycloalkyl,” “carbocyclic,” or “carbocycle” refers to hydrocarbon ringsystem having 3 to 20 overall number of ring atoms (e.g., 3-20 membered cycloalkyl is a cycloalkyl with 3 to 20 ring atoms, or C₃₋₂₀ cycloalkyl is a cycloalkyl with 3-20 carbon ring atoms) and for a 3-5 membered cycloalkyl being fully saturated or having no more than one double bond between ring vertices and for a 6 membered cycloalkyl or larger being fully saturated or having no more than two double bonds between ring vertices. As used herein, “cycloalkyl,” “carbocyclic,” or “carbocycle” is also meant to refer to bicyclic, polycyclic and spirocyclic hydrocarbon ring system, such as, for example, bicyclo[2.2.1]heptane, pinane, bicyclo[2.2.2]octane, adamantane, norborene, spirocyclic C₅₋₁₂alkane, etc. As used herein, the terms, “alkenyl,” “alkynyl,” “cycloalkyl,”, “carbocycle,” and “carbocyclic,” are meant to include mono and polyhalogenated variants thereof.

The term “heterocycloalkyl,” “heterocyclic,” or “heterocycle” refers to a saturated or partially unsaturated ring system radical having the overall having from 3-20 ring atoms (e.g., 3-20 membered heterocycloalkyl is a heterocycloalkyl radical with 3-20 ring atoms, a C₂₋₁₉ heterocycloalkyl is a heterocycloalkyl having 3-10 ring atoms with between 2-19 ring atoms being carbon) that contain from one to ten heteroatoms selected from N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and the like A “heterocycloalkyl,” “heterocyclic,” or “heterocycle” group can be attached to the remainder of the molecule through one or more ring carbons or heteroatoms. A “heterocycloalkyl,” “heterocyclic,” or “heterocycle” can include mono- and poly-halogenated variants thereof.

The terms “alkoxy,” and “alkylthio”, are used in their conventional sense, and refer to those alkyl groups attached to the remainder of the molecule via an oxygen atom (“oxy”) or thio grou, and further include mono- and poly-halogenated variants thereof.

The terms “halo” or “halogen,” by themselves or as part of another substituent, mean, unless otherwise stated, a fluorine, chlorine, bromine, or iodine atom. The term “(halo)alkyl” is meant to include both a “alkyl” and “haloalkyl” substituent. Additionally, the term “haloalkyl,” is meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, difluoromethyl, and the like.

The term “aryl” means a carbocyclic aromatic group having 6-14 carbon atoms, whether or not fused to one or more groups. Examples of aryl groups include phenyl, naphthyl, biphenyl and the like unless otherwise stated.

The term “heteroaryl” refers to aryl ring(s) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

The term saccharide includes monosaccharides, disaccharides and trisaccharides. The term includes glucose, sucrose fructose, galactose and ribose, as well as deoxy sugars such as deoxyribose and amino sugar such as galactosamine. Saccharide derivatives can conveniently be prepared as described in International Patent Applications Publication Numbers WO 96/34005 and 97/03995. A saccharide can conveniently be linked to the remainder of a compound of formula I through an ether bond, a thioether bond (e.g. an S-glycoside), an amine nitrogen (e.g., an N-glycoside), or a carbon-carbon bond (e.g. a C-glycoside). In one embodiment the saccharide can conveniently be linked to the remainder of a compound of formula I through an ether bond. In one embodiment the term saccharide includes a group of the formula:

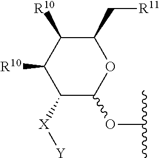

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy. In another embodiment the saccharide can be selected from the group consisting of:

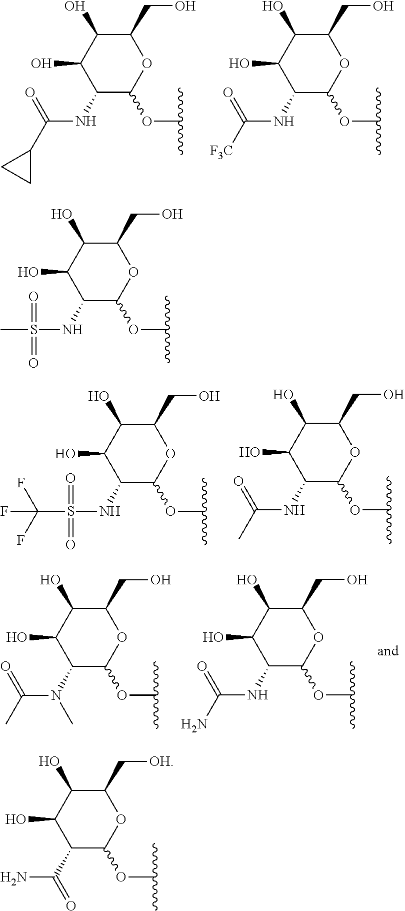

In another embodiment the saccharide can be:

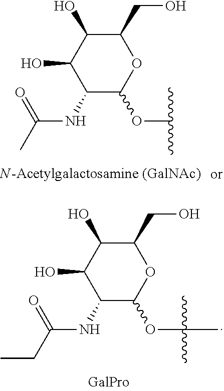 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

The term “animal” includes mammalian species, such as a human, mouse, rat, dog, cat, hamster, guinea pig, rabbit, livestock, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include triglycerides of various compositions as well as waxes; (2) “compound lipids,” which include phospholipids and glycolipids; and (3) “derived lipids” such as steroids.

The term “salts” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfate, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

The term “acyl” includes any alkyl, alkenyl, or alkynyl wherein the carbon at the point of attachment is substituted with an oxo group, as defined below. The following are non-limiting examples of acyl groups: —C(═O)alkyl, —C(═O)alkenyl, and —C(═O)alkynyl.

The term “fusogenic” refers to the ability of a lipid particle, such as a SNALP, to fuse with the membranes of a cell. The membranes can be either the plasma membrane or membranes surrounding organelles, e.g., endosome, nucleus, etc.

As used herein, the term “aqueous solution” refers to a composition comprising in whole, or in part, water.

As used herein, the term “organic lipid solution” refers to a composition comprising in whole, or in part, an organic solvent having a lipid.

“Distal site,” as used herein, refers to a physically separated site, which is not limited to an adjacent capillary bed, but includes sites broadly distributed throughout an organism.

“Serum-stable” in relation to nucleic acid-lipid particles such as SNALP means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of lipid particles that leads to a broad biodistribution of an active agent such as an siRNA within an organism. Some techniques of administration can lead to the systemic delivery of certain agents, but not others. Systemic delivery means that a useful, e.g., therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In one embodiment, systemic delivery of lipid particles is by intravenous delivery.

In certain embodiments, administration is subcutaneous.

In certain embodiments, administration is via subcutaneous injection.

In certain embodiments, administration is a weekly or montly subcutaneous injection.

In certain embodiments, administration is oral administration.

“Local delivery,” as used herein, refers to delivery of an active agent such as an siRNA directly to a target site within an organism. For example, an agent can be locally delivered by direct injection into a disease site, other target site, or a target organ such as the liver, heart, pancreas, kidney, and the like.

It will be appreciated by those skilled in the art that compounds having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities.

Unless otherwise specifically noted, when a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Unless stated otherwise herein, the term “about”, when used in connection with a value or range of values, means plus or minus 5% of the stated value or range of values.

Liver fibrosis is caused by the formation of an abnormally large amount of scar tissue in the liver. Liver fibrosis occurs when the liver attempts to repair and replace damaged cells. Various disorders and drugs can damage the liver and cause fibrosis.

Nonalcoholic fatty liver disease (NAFLD) is a condition in which triglycerides accumulate in the liver.

Nonalcoholic steatohepatitis (NASH) and alcoholic steatohepatitis (ASH) are advanced stages of non-alcoholic fatty liver disease (NAFLD) and alcoholic fatty liver disease (AFLD). NASH is a type of NAFLD.

NASH is associated with inflammatory changes and liver cell damage. NASH is a leading cause of liver disease and often progresses to liver fibrosis, cirrhosis and hepatocellular carcinoma (HCC).

ASH is a chronic, progressive liver disease characterized by fibrosis of the liver as well as possible necrosis of the liver tissue, brought on by excessive, prolonged alcohol use. Women are more susceptible to the disease because alcohol metabolism is lower in women than in men.

### Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNA are chemically synthesized. The oligonucleotides that comprise the siRNA molecules can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, Calif.). However, a larger or smaller scale of synthesis is also within the scope. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

### Embodiments

Another aspect provides a composition comprising a double stranded siRNA molecule described herein, or a sense or antisense strand thereof.

In one embodiment, the composition is a pharmaceutical composition that comprises a pharmaceutically acceptable carrier.

One aspect is a compound of formula I, as set forth about herein, or a salt thereof.

In one embodiment of the compound of formula I, R¹ a is targeting ligand;

L¹ is absent or a linking group;

L² is absent or a linking group;

R² is a double stranded siRNA molecule selected from the double stranded siRNA described herein, e.g., in FIG. 1;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂alkyl-OR^{B} and C₁₋₈alkyl that is optionally substituted with one or more groups independently selected from halo, hydroxy, and C₁₋₃alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10.

In one embodiment R¹ is —C(H)₍₃₋ₚ₎(L³-saccharide)ₚ, wherein each L³ is independently a linking group; p is 1, 2, or 3; and saccharide is a monosaccharide or disaccharide.

In one embodiment the saccharide is:

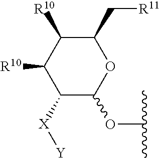

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy;

or a salt thereof.

In one embodiment the saccharide is selected from the group consisting of:

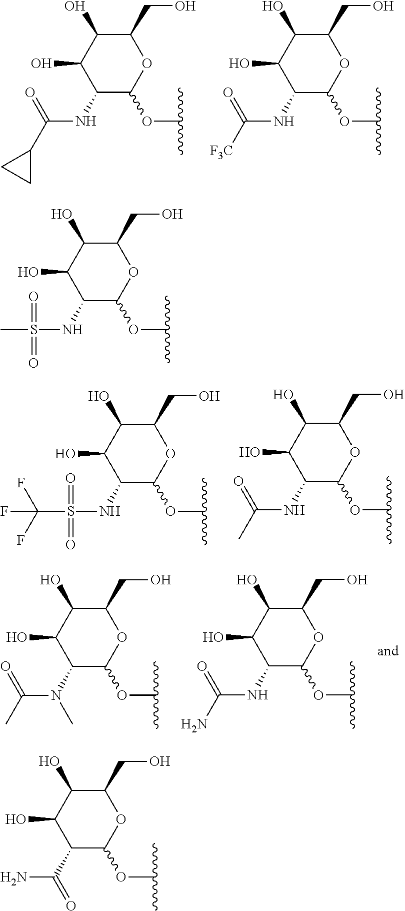

and salts thereof.

In one embodiment the saccharide is:

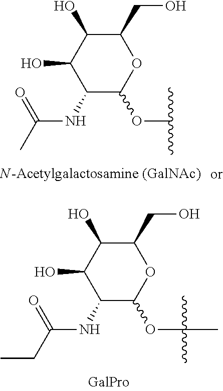 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment each L³ is independently a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X}is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment each L³ is independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X}is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L³ is:

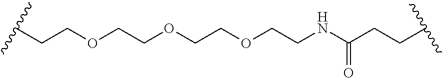 [SMILES: *CCOCCOCCOCCNC(=O)CC*]

or a salt thereof.

In one embodiment R¹ is:

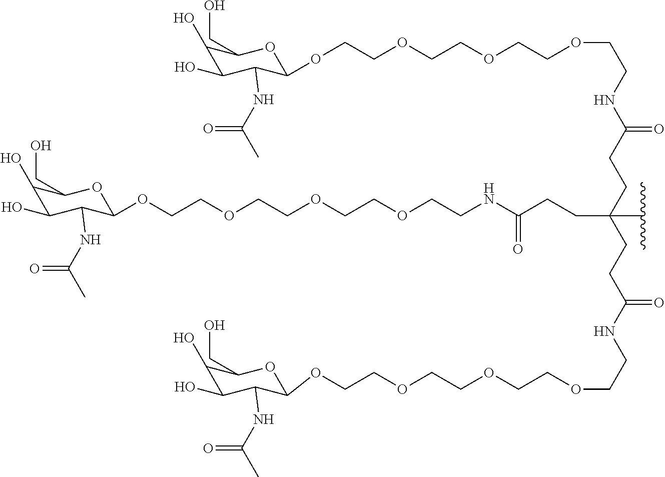 [SMILES: *C(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

or a salt thereof.

In one embodiment R¹ is:

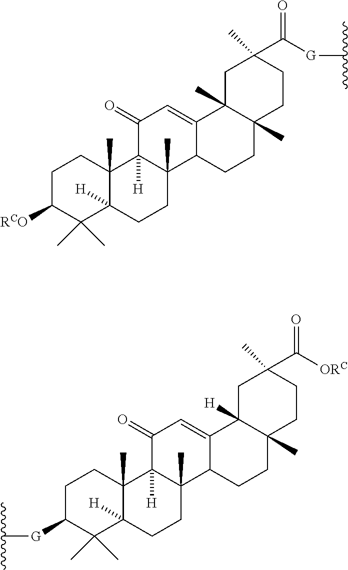

wherein G is —NH— or —O—;

R^{C} is hydrogen, (C₁-C₈)alkyl, (C₁-C₈)haloalkyl, (C₁-C₈)alkoxy, (C₁-C₆)alkanoyl, (C₃-C₂₀)cycloalkyl, (C₃-C₂₀)heterocycle, aryl, heteroaryl, monosaccharide, disaccharide or trisaccharide; and wherein the cycloalkyl, heterocyle, ary, heteroaryl and saccharide are optionally substituted with one or more groups independently selected from the group consisting of halo, carboxyl, hydroxyl, amino, (C₁-C₄)alkyl, (C₁-C₄)haloalkyl, (C₁-C₄)alkoxy and (C₁-C₄)haloalkoxy;

or a salt thereof.

In one embodiment R^{C} is:

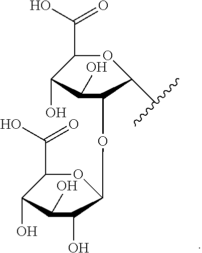

In one embodiment R¹ is:

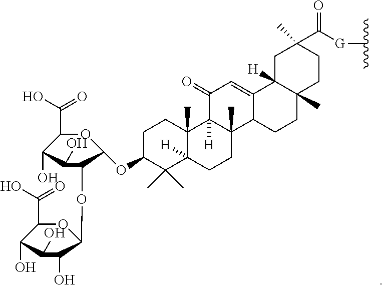

In one embodiment R^{C} is:

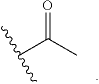 [SMILES: *C(C)=O]

In one embodiment G is —NH—.

In one embodiment R¹ is:

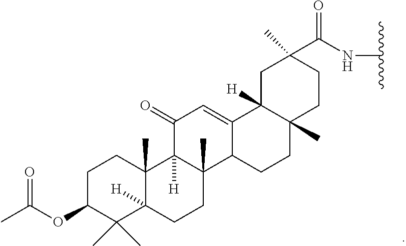

In one embodiment R¹ is:

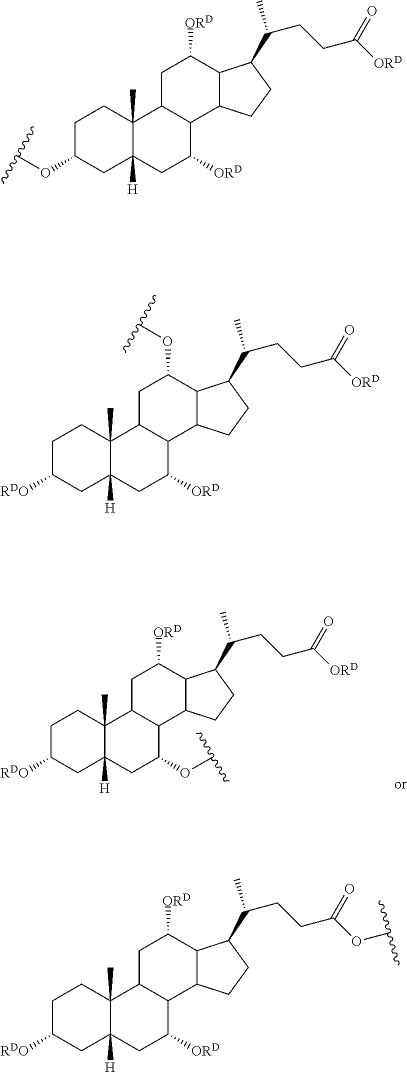

wherein each R^{D} is independently selected from the group consisting of hydrogen, (C₁-C₆)alkyl, (C₉-C₂₀)alkylsilyl, (R^{W})₃Si—, (C₂-C₆)alkenyl, tetrahydropyranyl, (C₁-C₆)alkanoyl, benzoyl, aryl(C₁-C₃)alkyl, TMTr (Trimethoxytrityl), DMTr (Dimethoxytrityl), MMTr (Monomethoxytrityl), and Tr (Trityl); and

each R^{W} is independently selected from the group consisting of (C₁-C₄)alkyl and aryl.

In one embodiment linking groups L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ and L² are independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ and L² are independently, a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

In one embodiment L₂ is connected to R₂ through —O—.

In one embodiment L¹ is selected from the group consisting of:

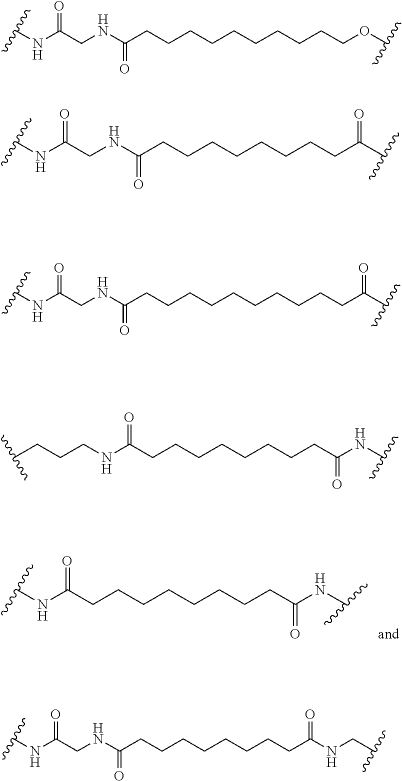 [SMILES: *NC(=O)CNC(=O)CCCCCCCCCCO* ; *NC(=O)CNC(=O)CCCCCCCCC(*)=O ; *NC(=O)CNC(=O)CCCCCCCCCCC(*)=O ; *CCCNC(=O)CCCCCCCCC(=O)N* ; *NC(=O)CCCCCCCCC(=O)N* ; *CNC(=O)CCCCCCCCC(=O)NCC(=O)N*]

In one embodiment L¹ is selected from the group consisting of:

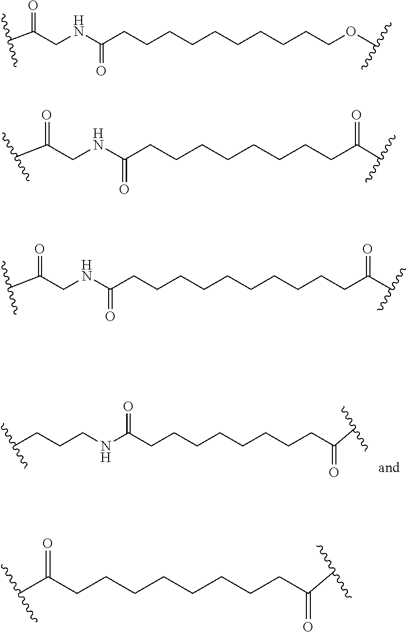 [SMILES: *OCCCCCCCCCCC(=O)NCC(*)=O ; *C(=O)CCCCCCCCC(=O)NCC(*)=O ; *C(=O)CCCCCCCCCCC(=O)NCC(*)=O ; *CCCNC(=O)CCCCCCCCC(*)=O ; *C(=O)CCCCCCCCC(*)=O]

and salts thereof.

In one embodiment L² is —CH₂—O— or —CH₂—CH₂—O—.

In one embodiment a compound of formula I has the following formula Ia:

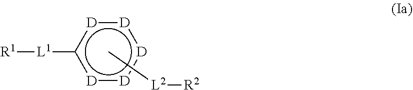

wherein:

each D is independently selected from the group consisting of

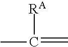

### and —N═;

or a salt thereof.

In one embodiment a compound of formula Ia is selected from the group consisting of:

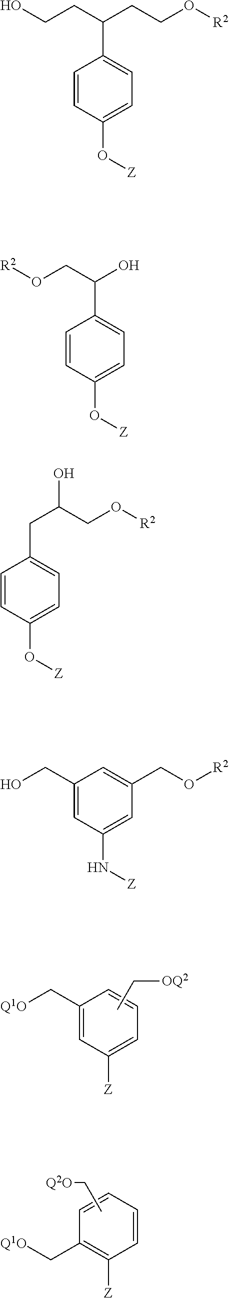

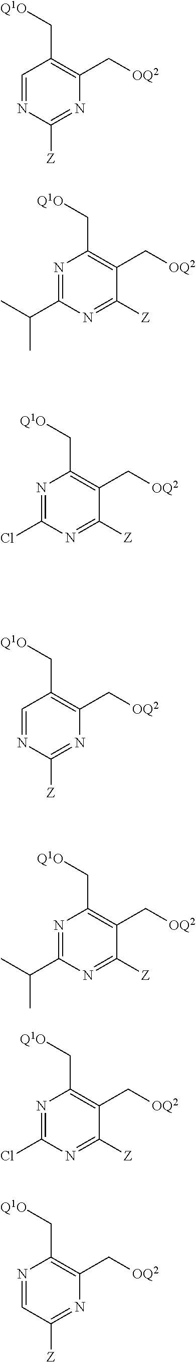

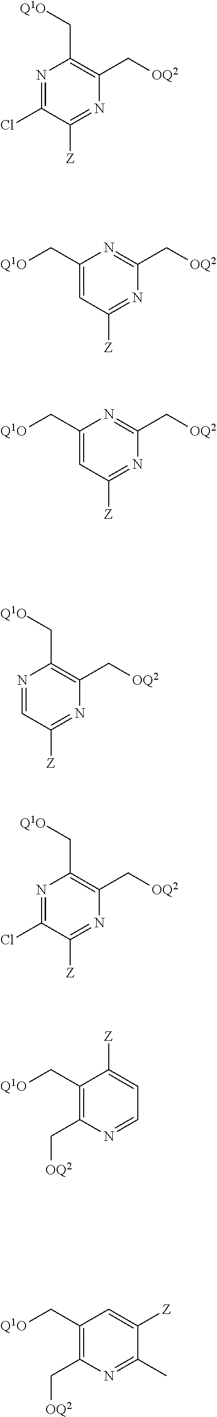

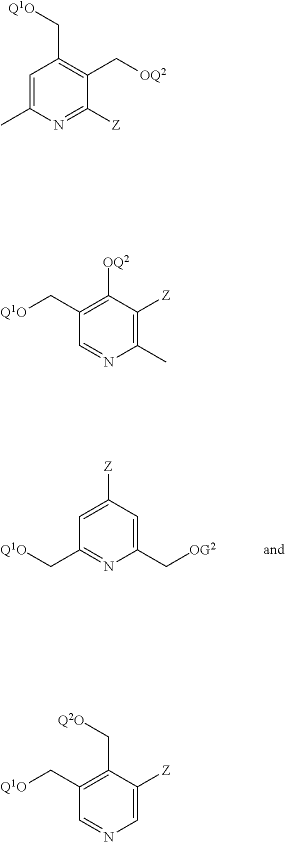

wherein:

Q¹ is hydrogen and Q² is R²; or Q¹ is R² and Q² is hydrogen;

Z is -L¹-R¹;

and salts thereof.

In one embodiment a compound of formula I has the following formula Ib:

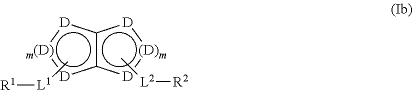

wherein:

each D is independently selected from the group consisting of

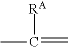

### and —N═;

each m is independently 1 or 2; or a salt thereof.

In one embodiment a compound of formula Ib is selected from the group consisting of:

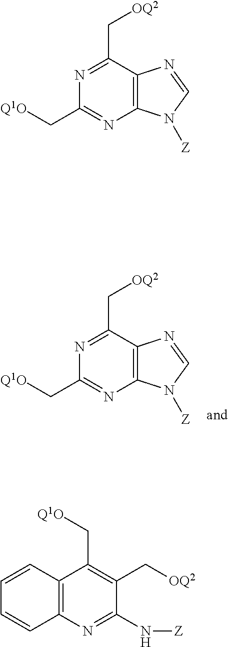

wherein:

Q¹ is hydrogen and Q² is R²; or Q¹ is R² and Q² is hydrogen;

Z is -L¹-R¹;

and salts thereof.

In one embodiment a compound of formula I has the following formula (Ic):

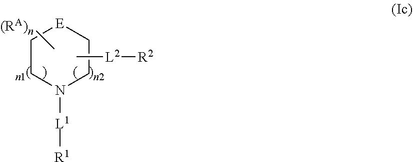

wherein E is —O— or —CH₂—;

n is selected from the group consisting of 0, 1, 2, 3, and 4; and

n1 and n2 are each independently selected from the group consisting of 0, 1, 2, and 3;

or a salt thereof.

In certain embodiments a compound of formula (Ic) is selected from the group consisting of:

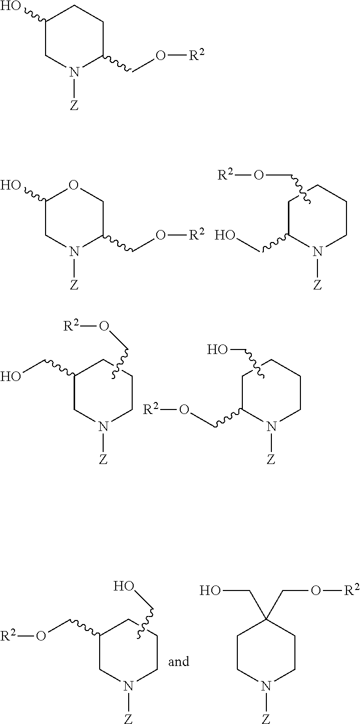

wherein Z is -L¹-R¹;

and salts thereof.

In one embodiment the -A-L²-R² moiety is:

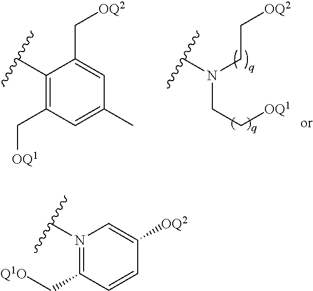

wherein:

Q¹ is hydrogen and Q² is R²; or Q¹ is R² and Q² is hydrogen; and

each q is independently 0, 1, 2, 3, 4 or 5;

or a salt thereof.

In one embodiment a compound of formula (I) is selected from the group consisting of:

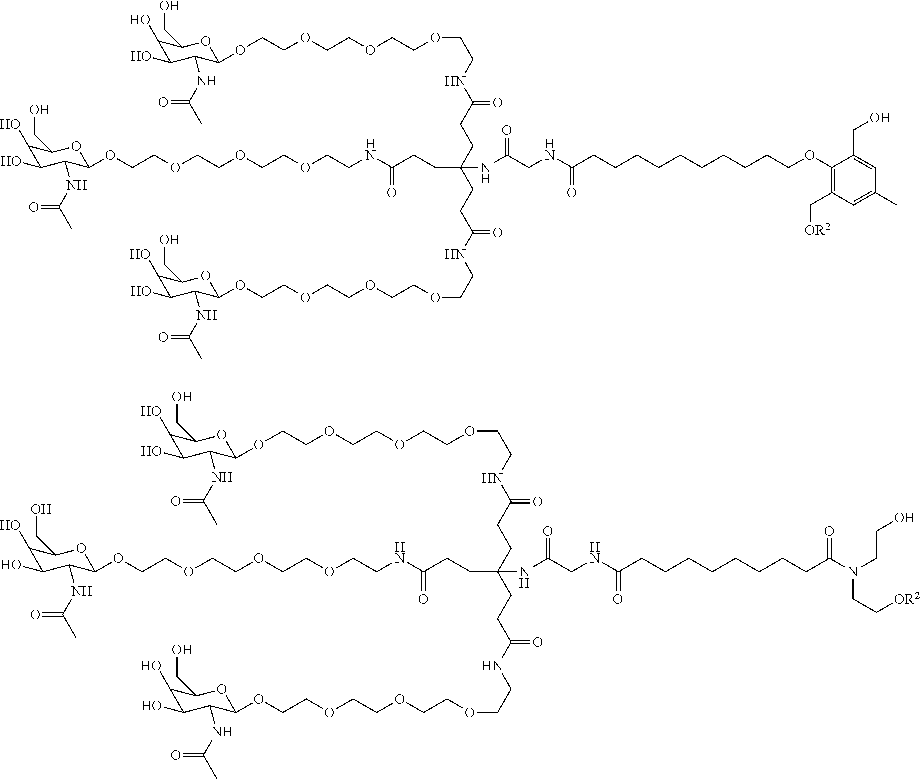

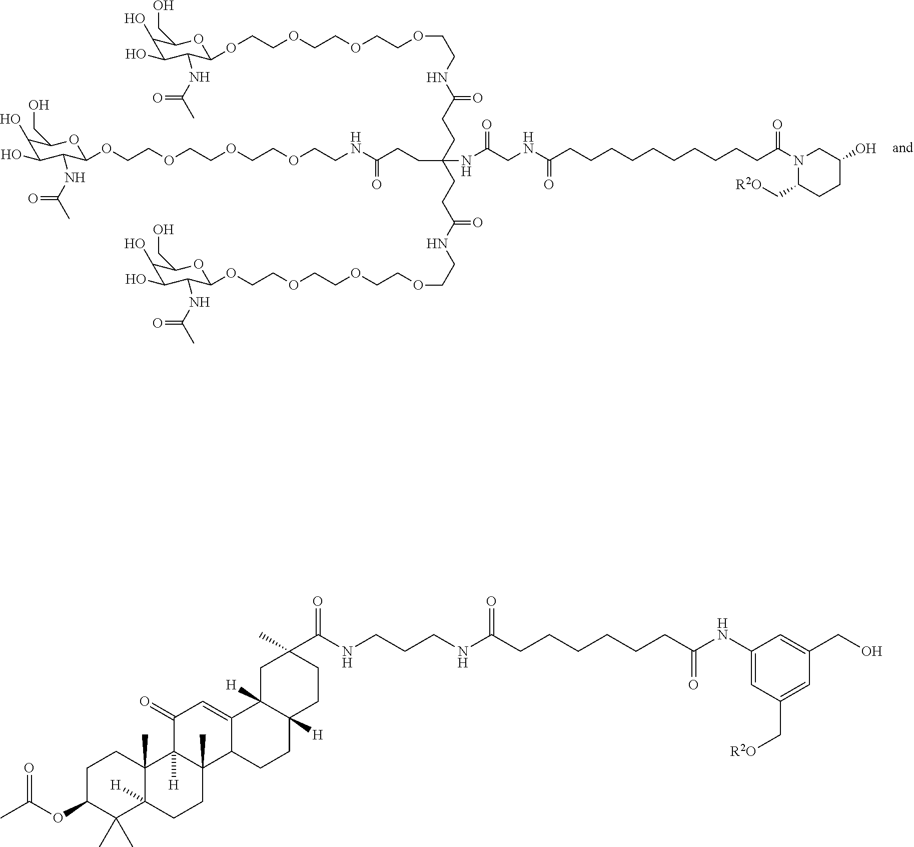

and salts thereof.

In one embodiment R¹ is selected from the group consisting of:

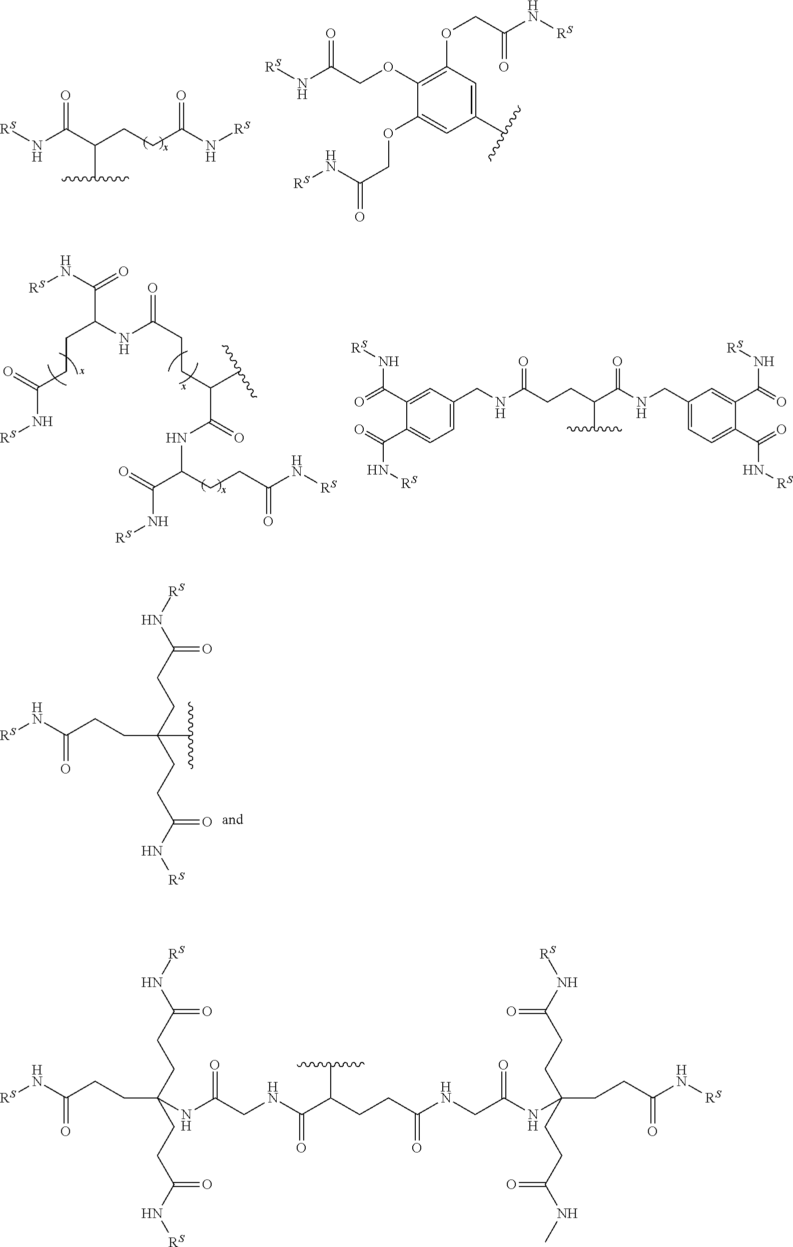

wherein R^{S} is

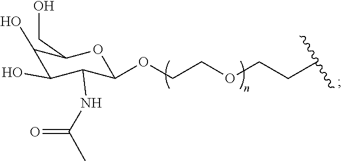

n is 2, 3, or 4;

x is 1 or 2.

In one embodiment L¹ is selected from the group consisting of:

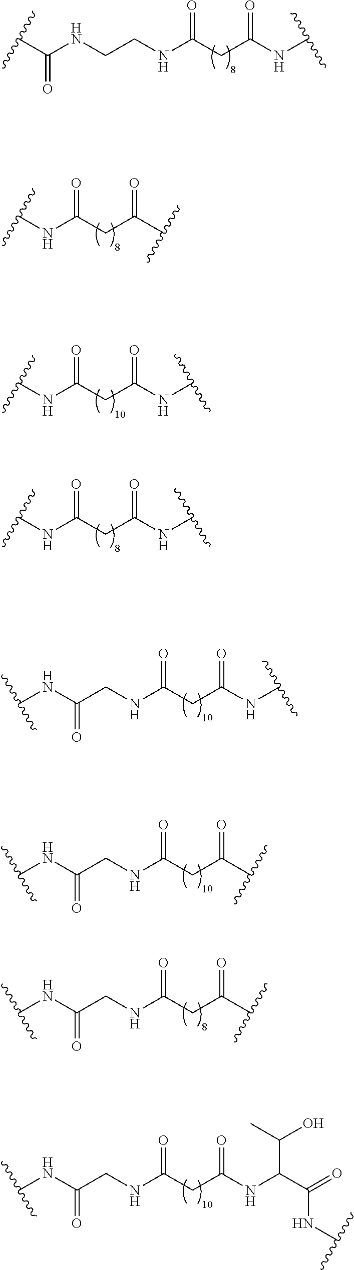

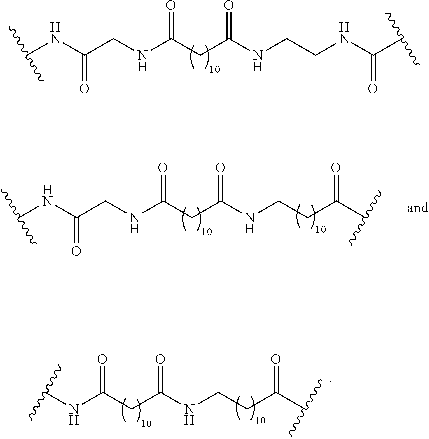

In one embodiment L¹ is selected from the group consisting of:

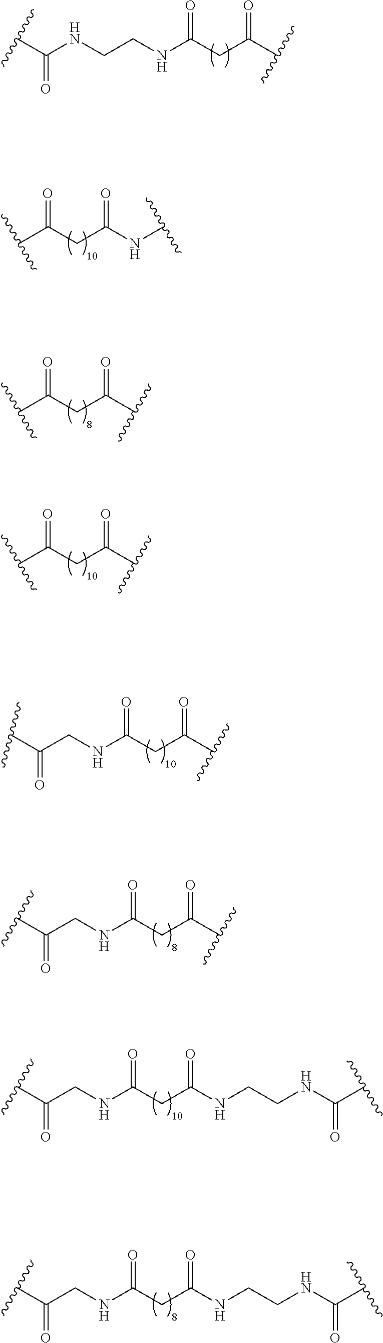

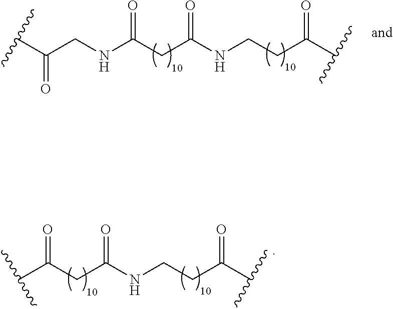

In one embodiment A is absent, phenyl, pyrrolidinyl, or cyclopentyl.

In one embodiment L² is C₁₋₄alkylene-O— that is optionally substituted with hydroxy.

In one embodiment L² is —CH₂O—, —CH₂CH₂O—, or —CH(OH)CH₂O—.

In one embodiment each R^{A} is independently hydroxy or C₁₋₈alkyl that is optionally substituted with hydroxyl.

In one embodiment each R^{A} is independently selected from the group consisting of hydroxy, methyl and —CH₂OH.

In one embodiment a compound of formula I has the following formula (Ig):

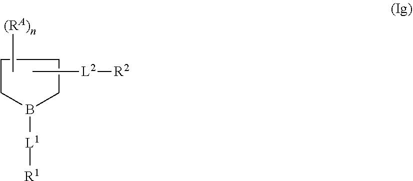

wherein B is —N— or —CH—;

L¹ is absent or —NH—;

L² is C₁₋₄alkylene-O— that is optionally substituted with hydroxyl or halo;

n is 0, 1, or 2;

or a salt thereof.

In one embodiment a compound of formula I has the following formula (Ig):

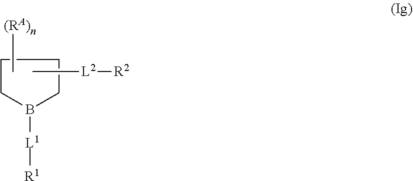

wherein B is —N— or —CH—;

L¹ is absent or —NH—;

L² is C₁₋₄alkylene-O— that is optionally substituted with hydroxyl or halo;

n is 0, 1, 2, 3, 4, 5, 6, or 7;

or a salt thereof.

In one embodiment a compound of formula I has the following formula (Ig):

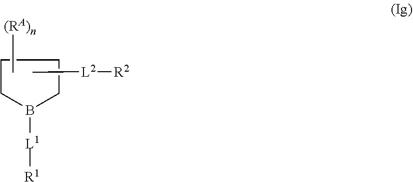

wherein B is —N— or —CH—;

L¹ is absent or —NH—;

L² is C1-4alkylene-O— that is optionally substituted with hydroxyl or halo;

n is 0, 1, 2, 3, or 4;

or a salt thereof.

In one embodiment a compound of formula Ig is selected from the group consisting of:

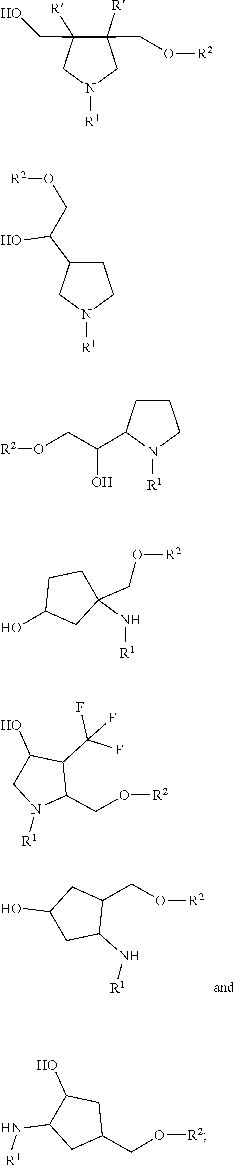

wherein R¹ is C₁₋₉alkyl, C₂₋₉alkenyl or C₂₋₉alkynyl; wherein the C₁₋₉alkyl, C₂₋₉alkenyl or C₂₋₉alkynyl are optionally substituted with halo or hydroxyl;

and salts thereof.

In one embodiment a compound of formula I is selected from the group consisting of:

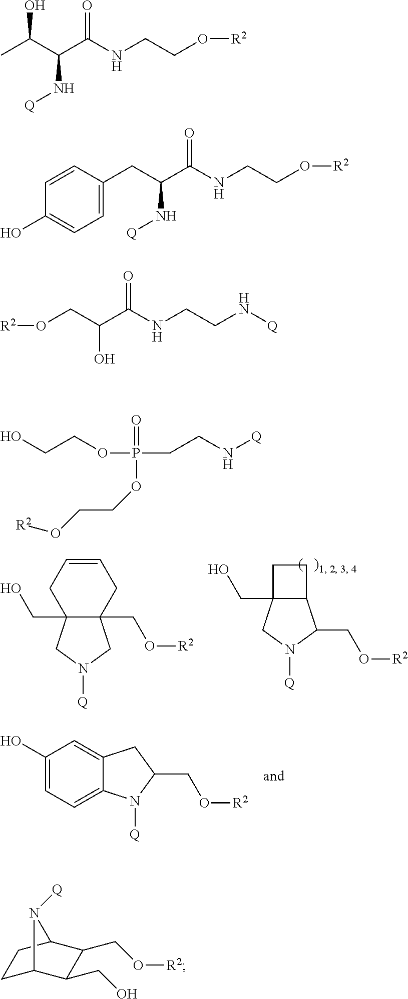

and salts thereof.

In one embodiment the compound of formula I or the salt thereof is selected from the group consisting of:

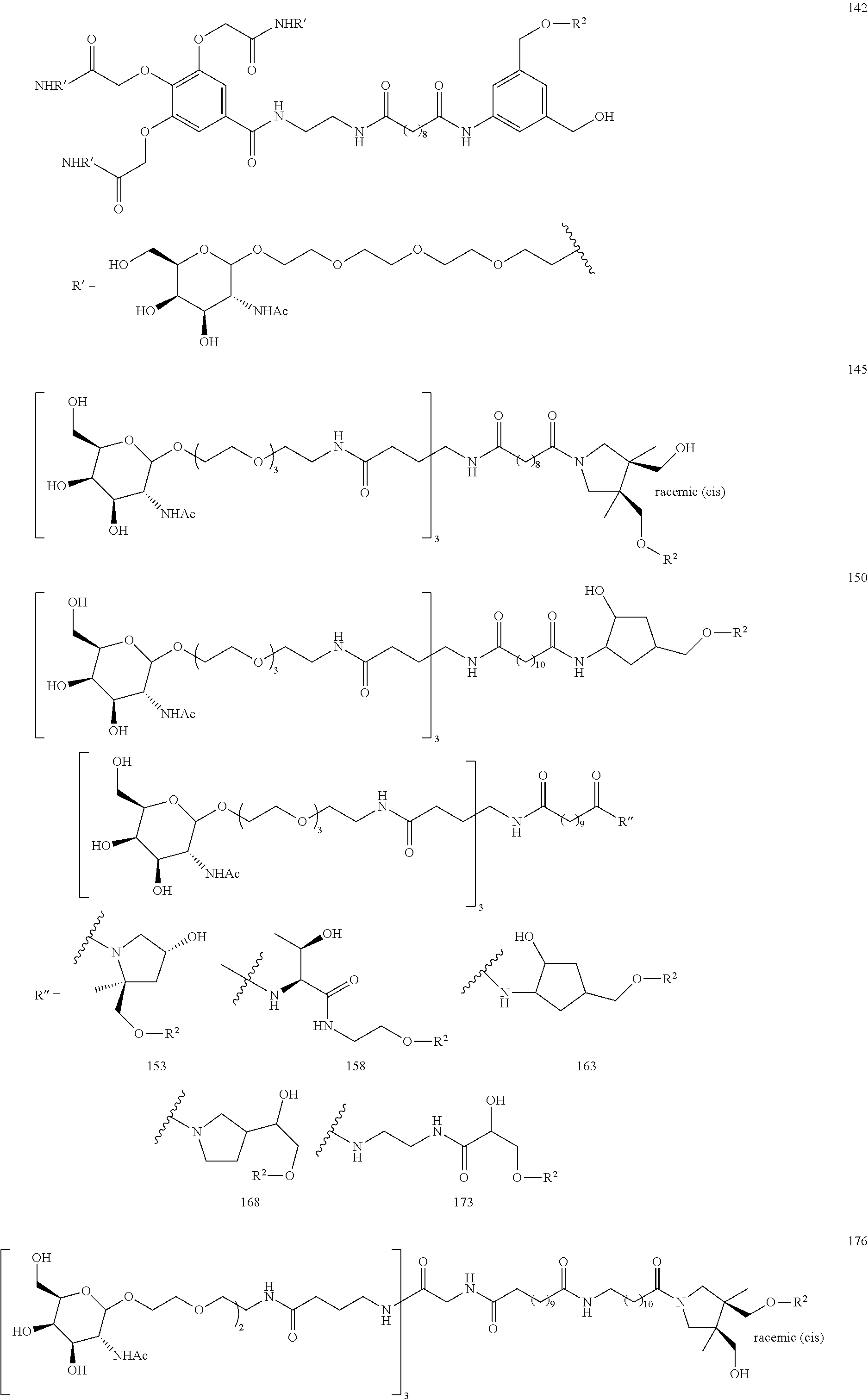

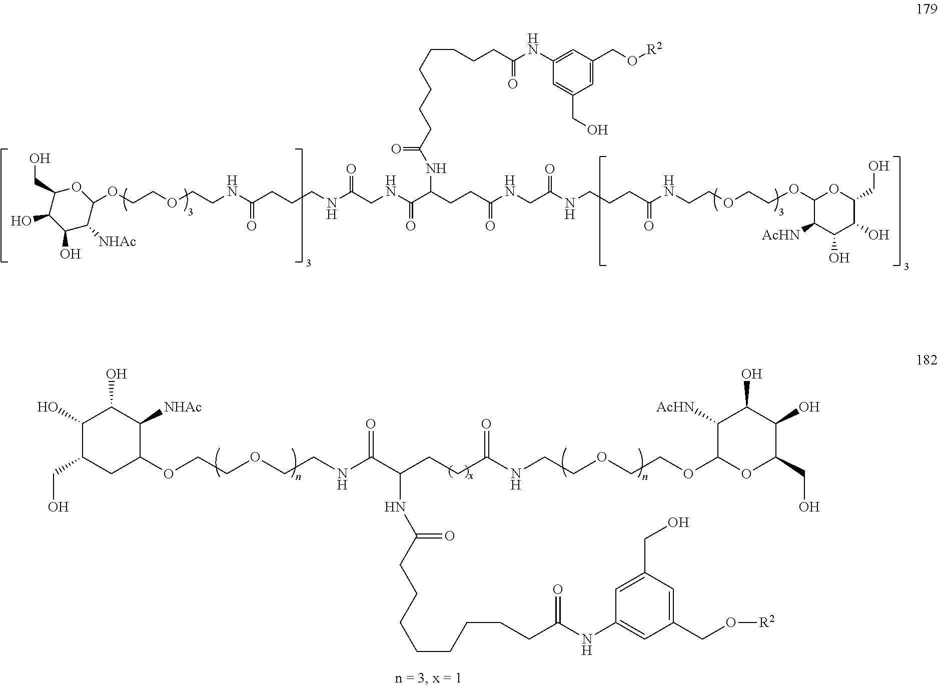

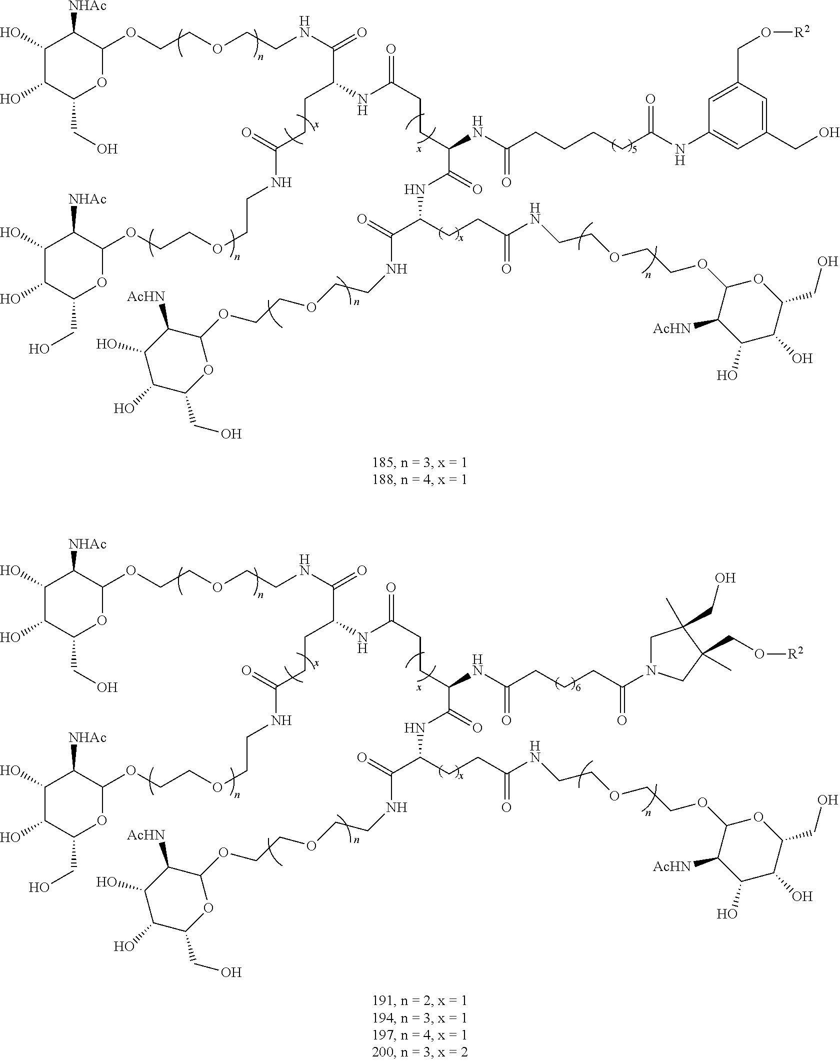

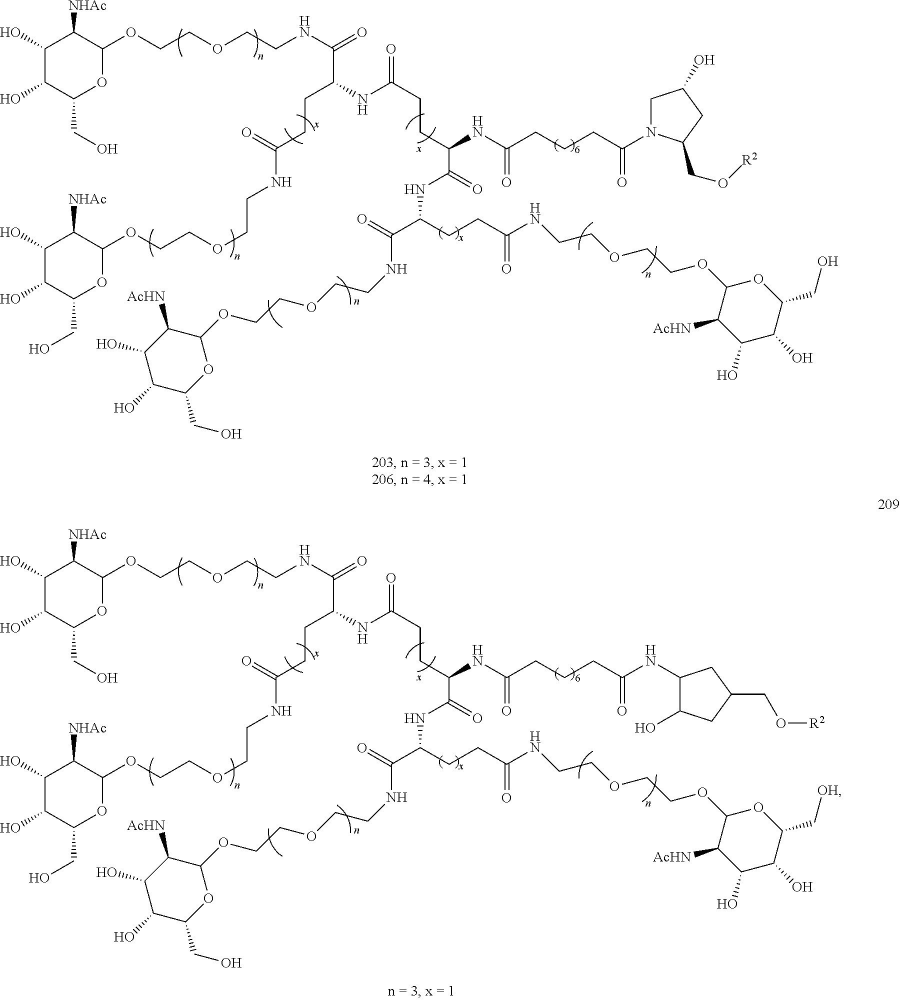

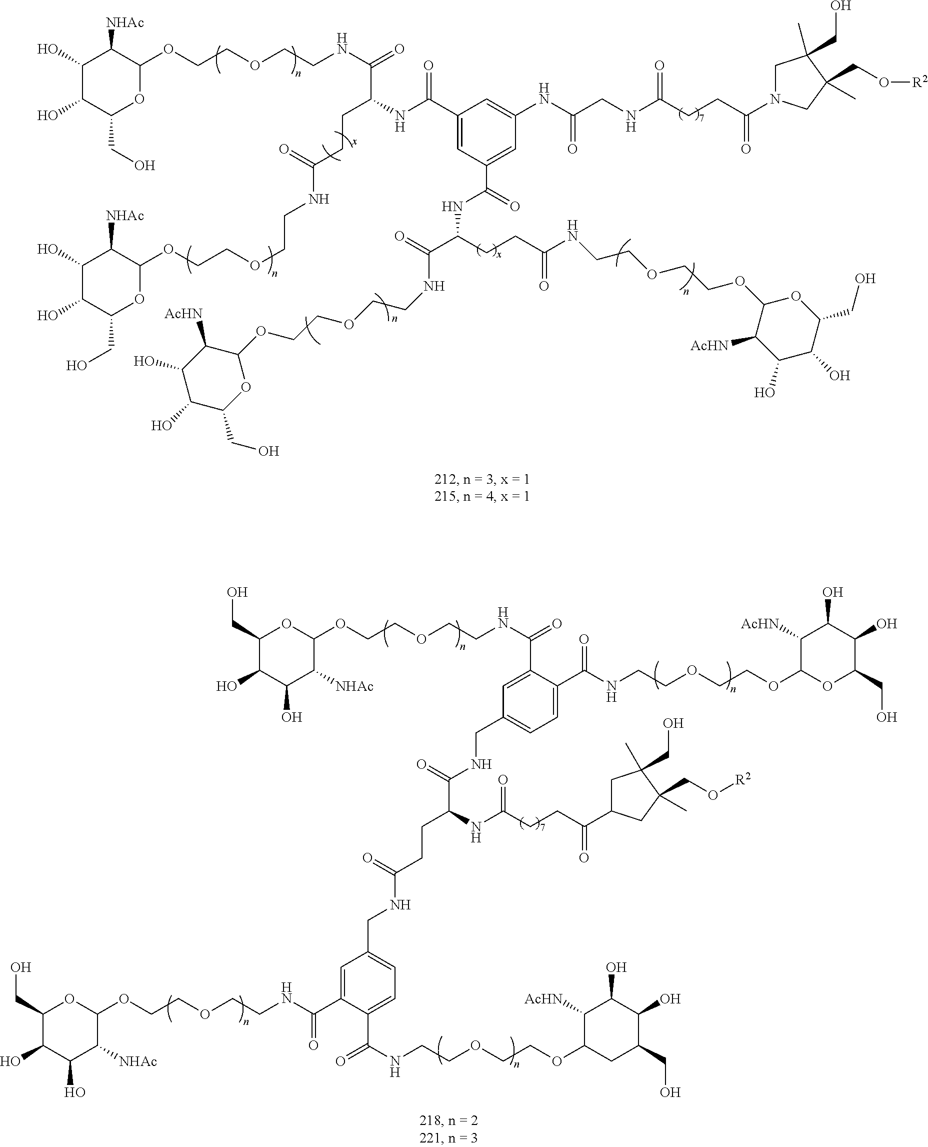

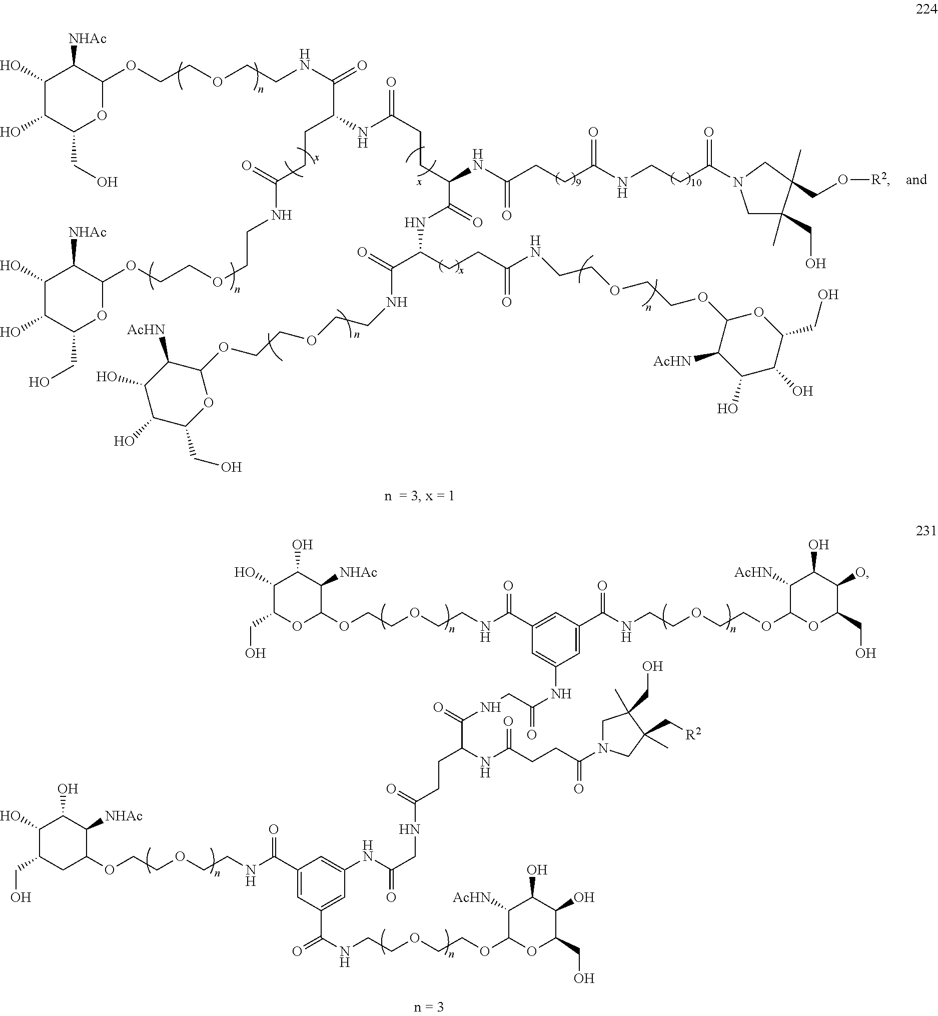

In one embodiment the compound of formula I or the salt thereof is selected from the group consisting of:

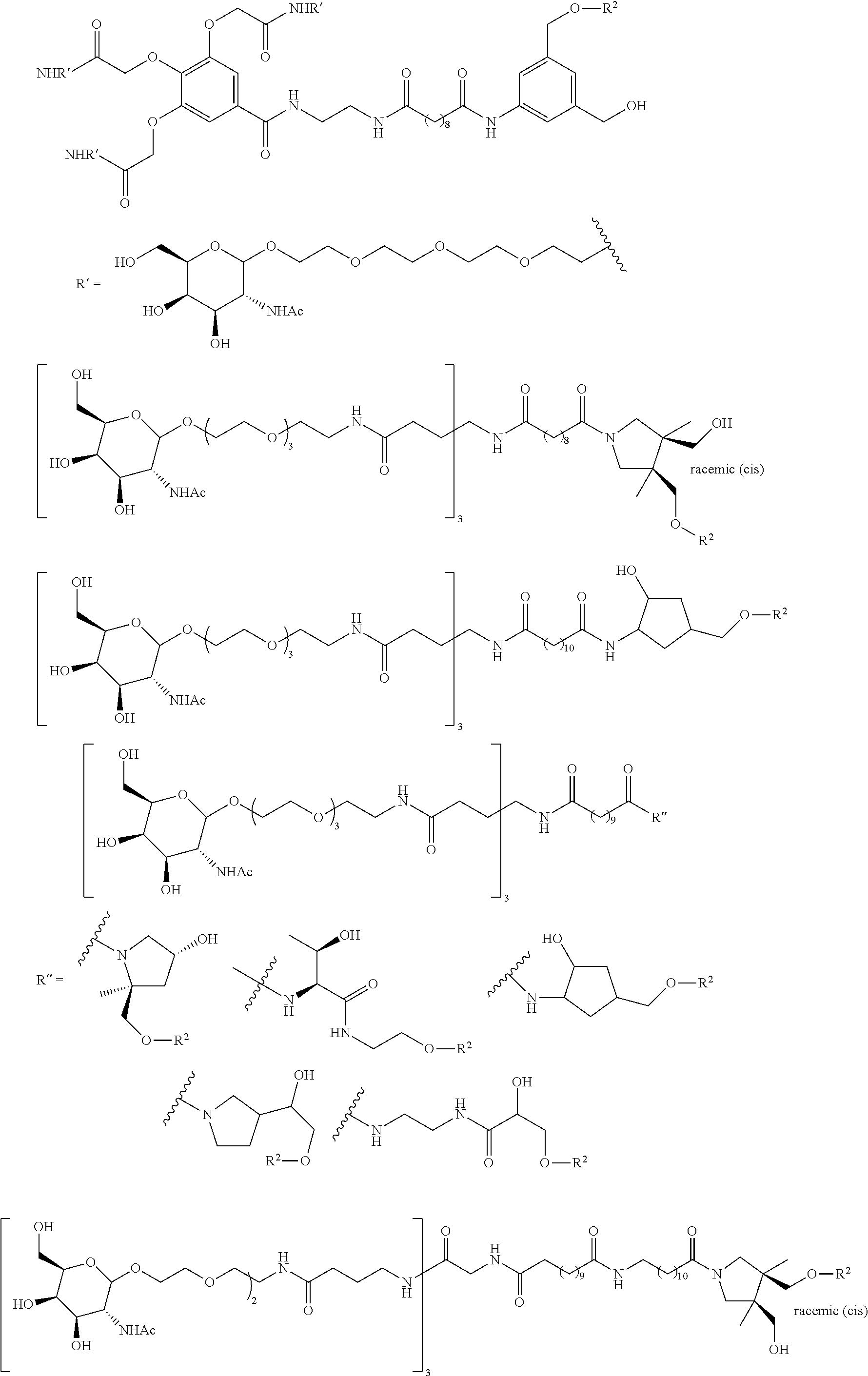

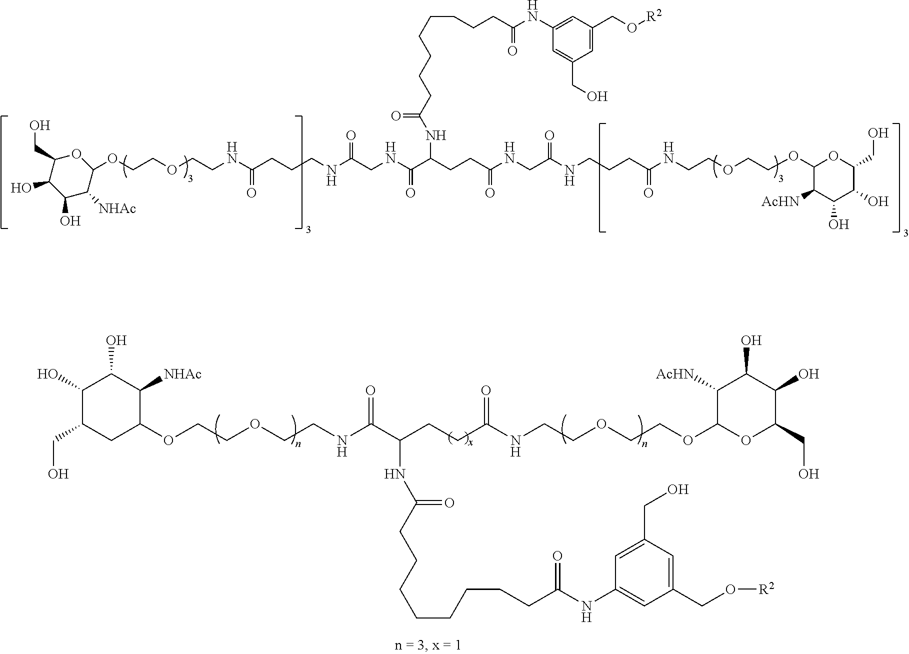

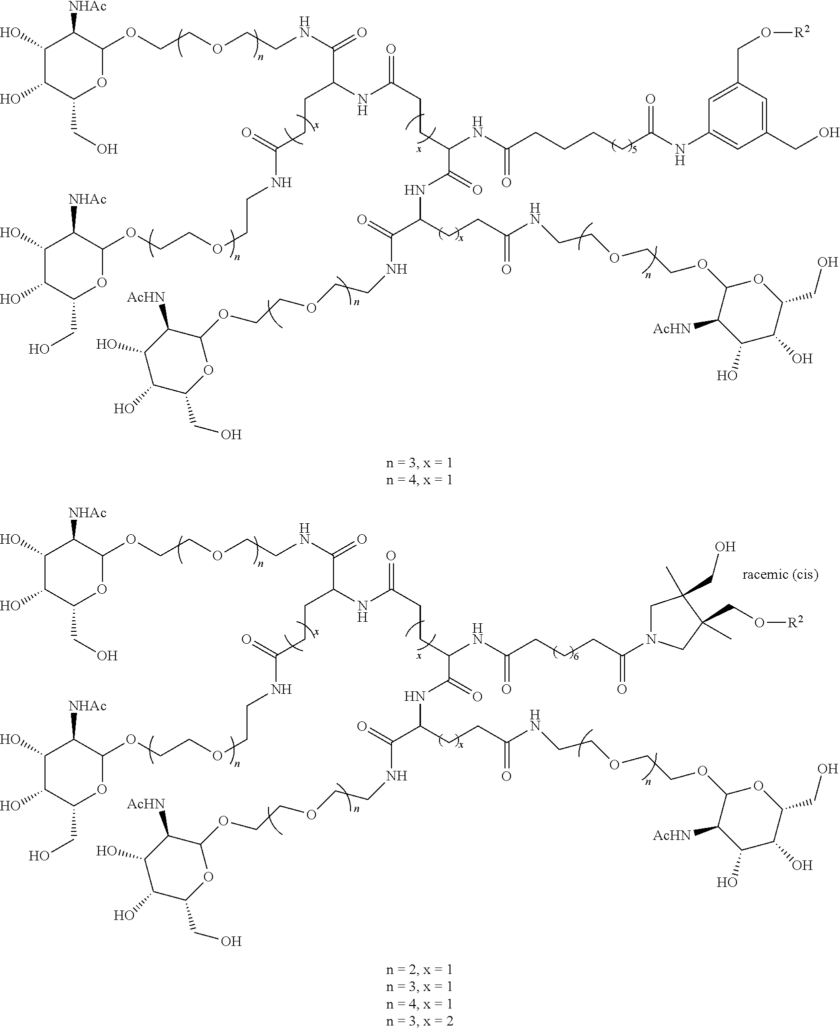

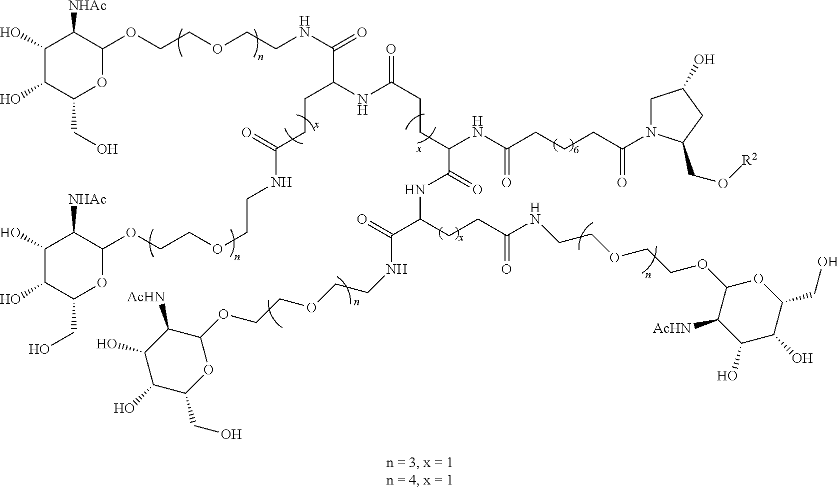

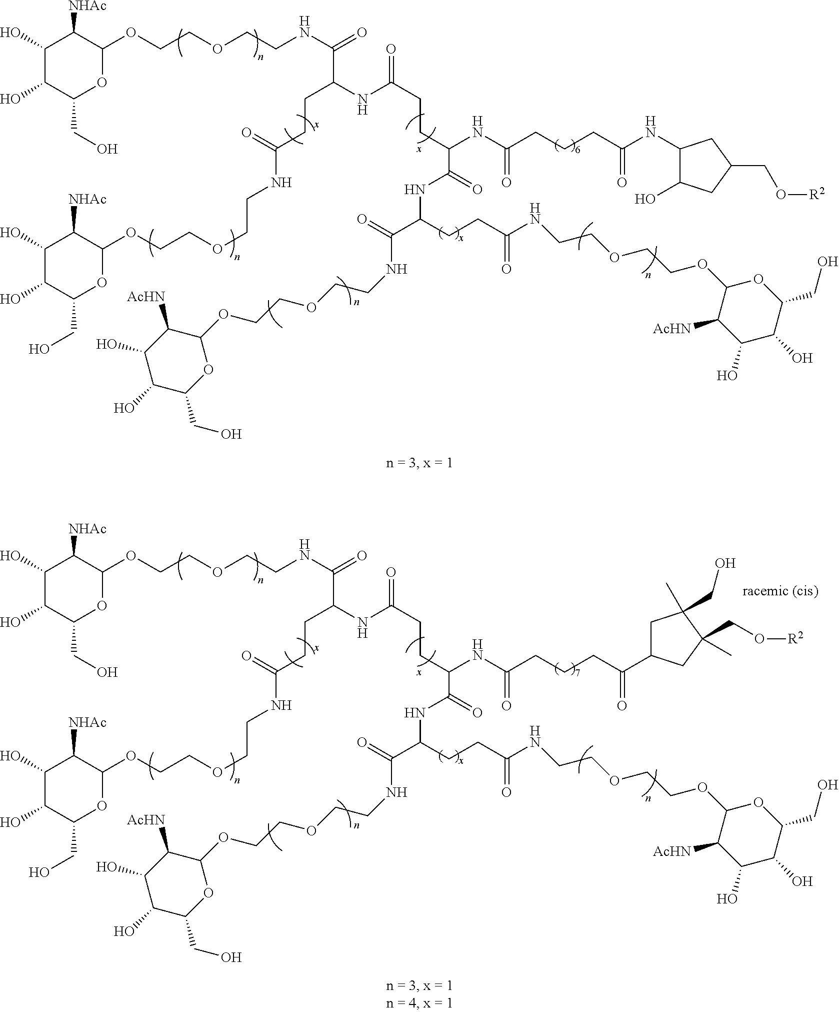

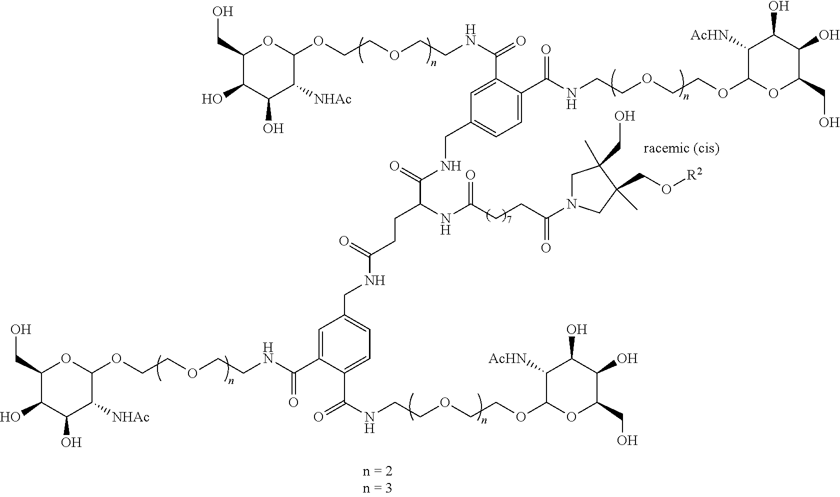

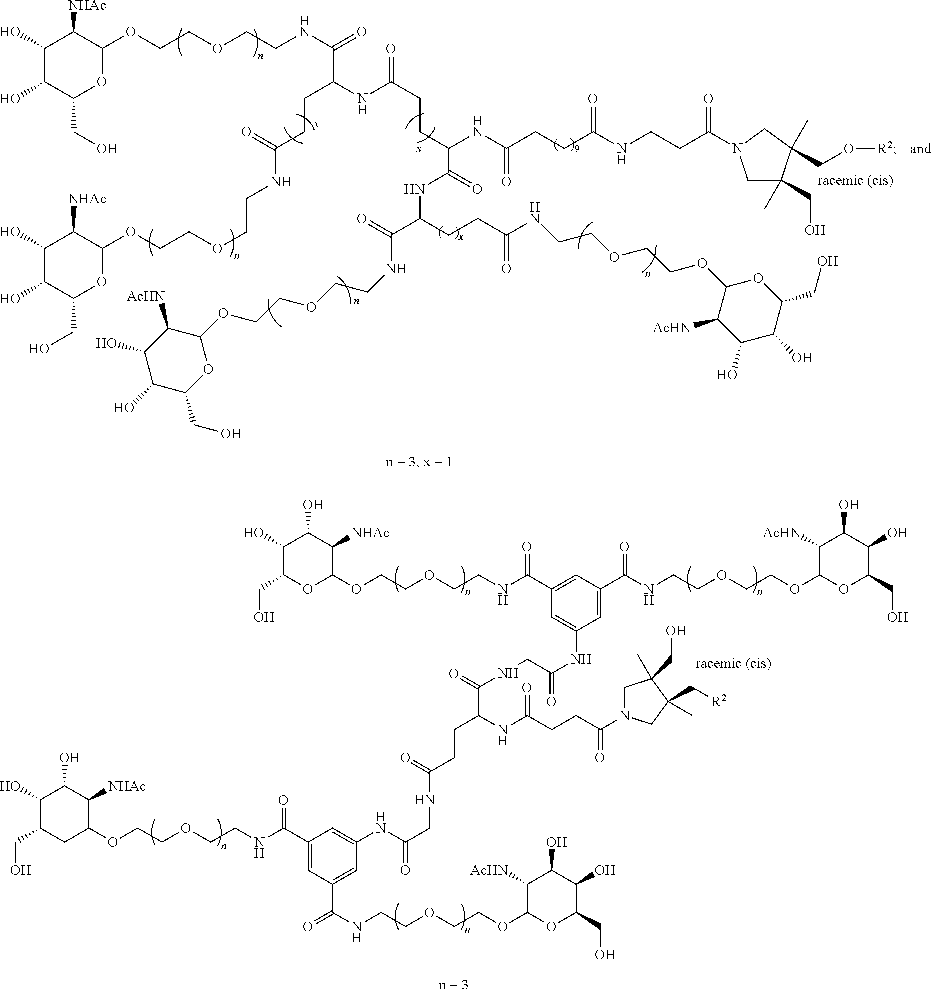

or pharmaceutically acceptable salts thereof, wherein R² is a double stranded siRNA molecule selected from the double stranded siRNA molecules disclosed herein, e.g., in FIG. 1.

In one embodiment the compound of formula I is:

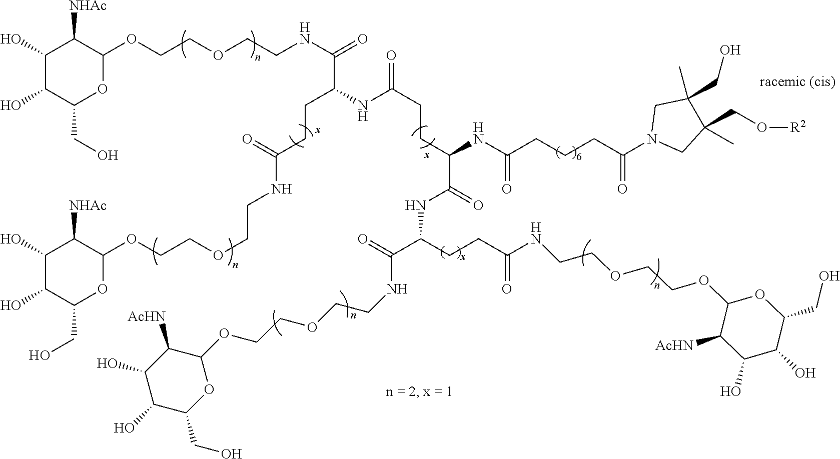

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

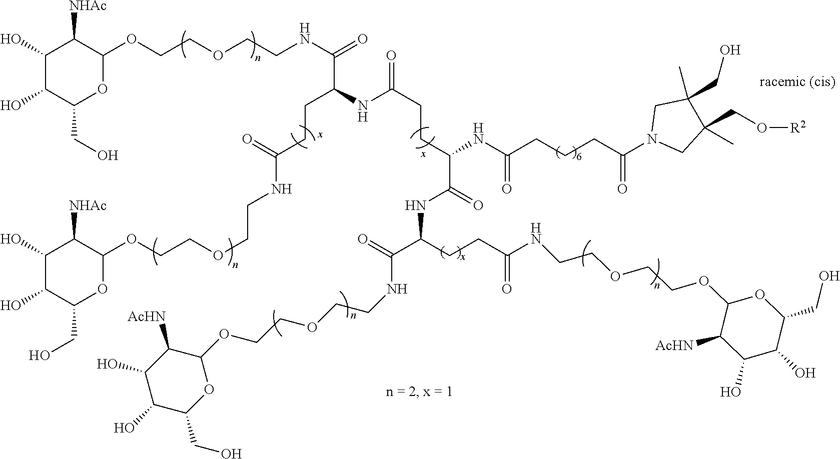

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

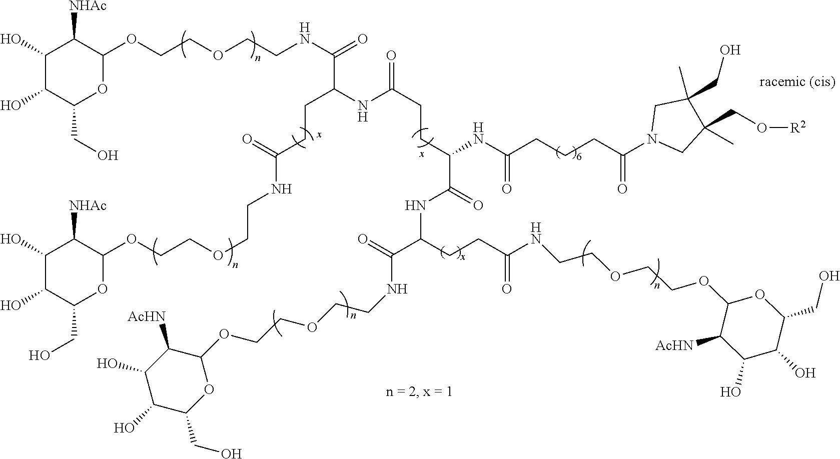

or a pharmaceutically acceptable salt thereof, wherein R² is a double stranded siRNA molecule (e.g. a double stranded siRNA molecule selected from the double stranded siRNA molecules disclosed herein, e.g., in FIG 1).

In one embodiment the compound of formula I is:

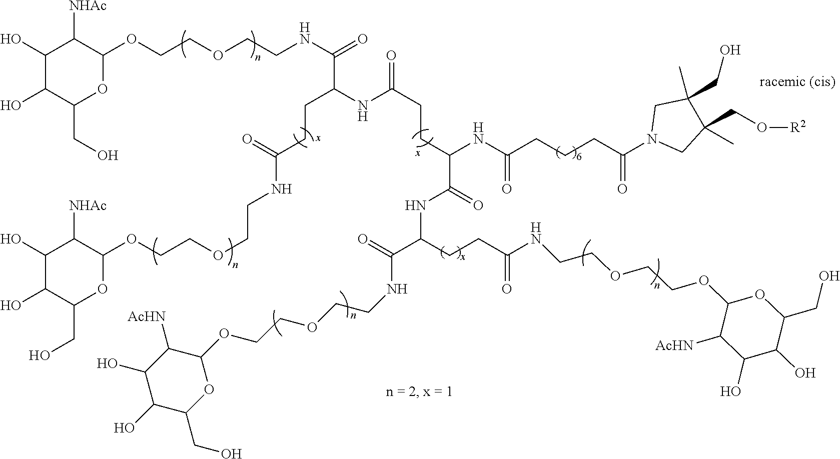

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

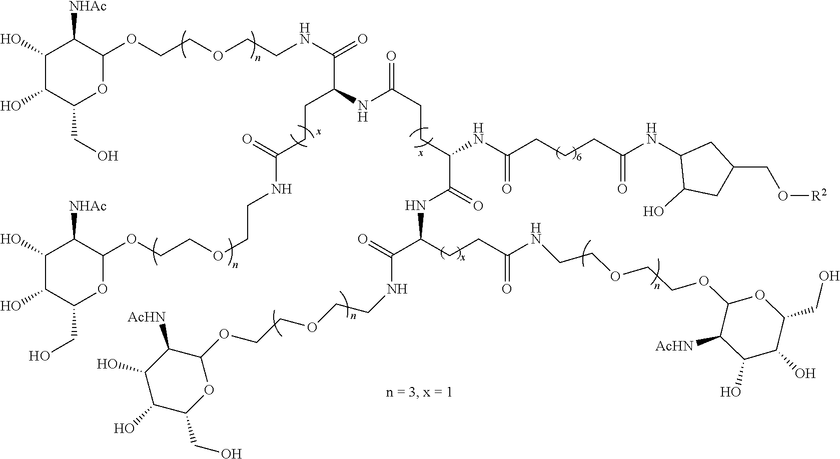

or a pharmaceutically acceptable salt thereof, wherein R² is a double stranded siRNA molecule (e.g. a double stranded siRNA molecule selected from the double stranded siRNA molecules disclosed herein, e.g., in FIG 1).

In one embodiment the compound of formula I is:

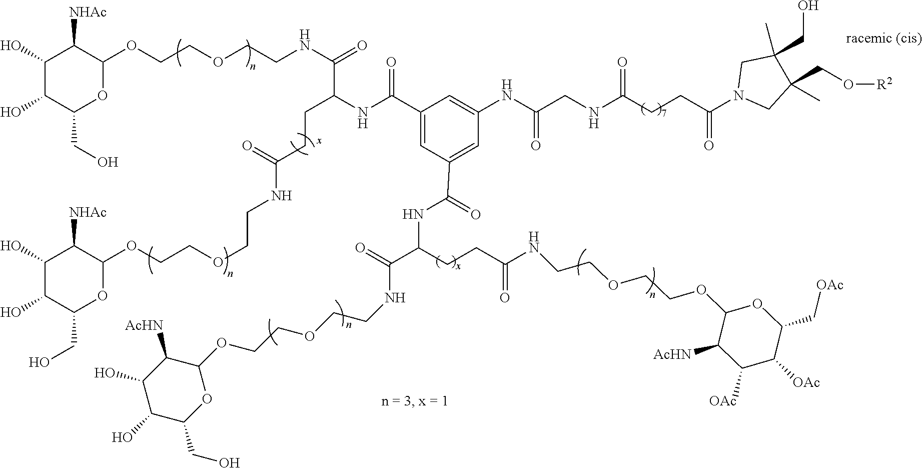

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

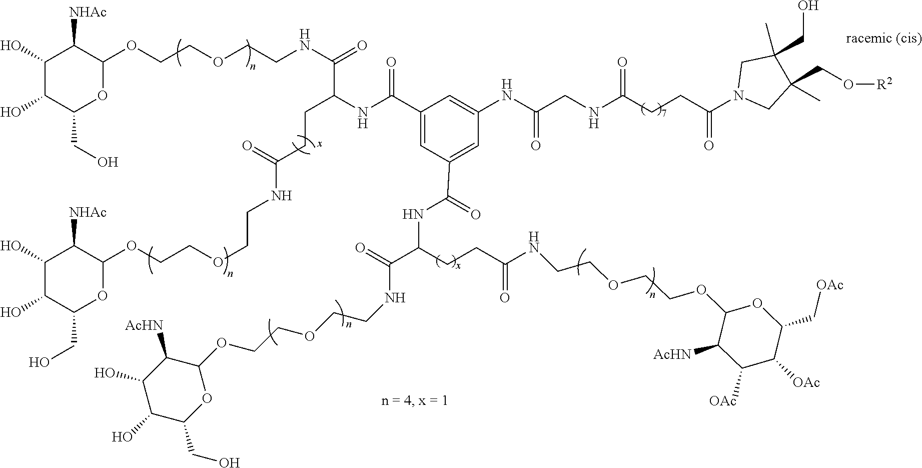

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

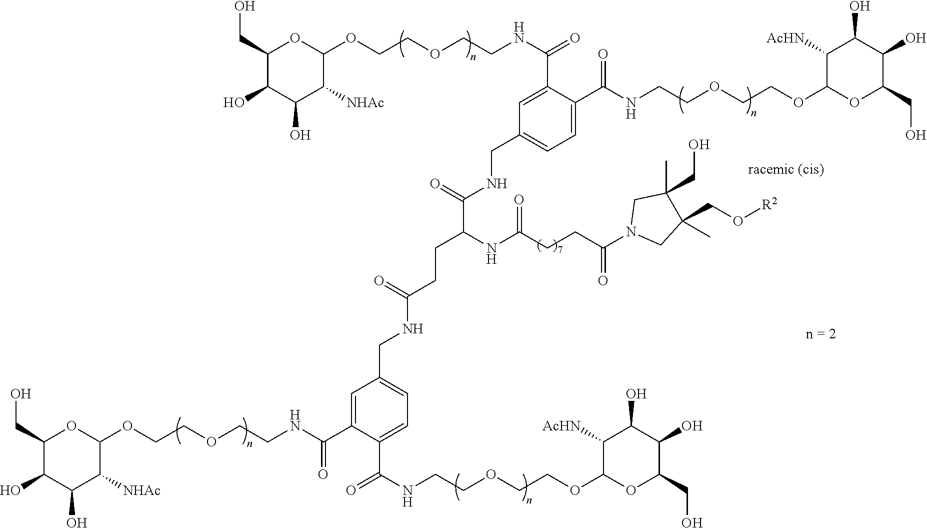

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

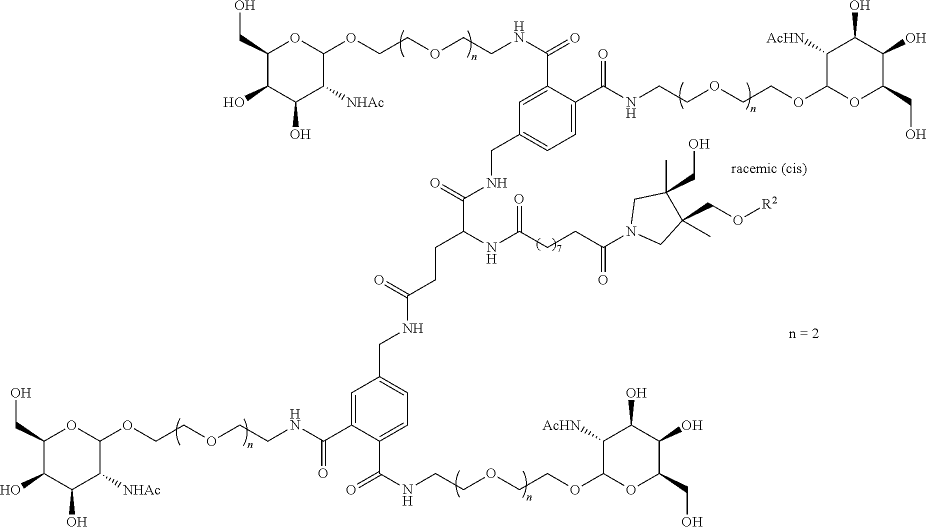

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

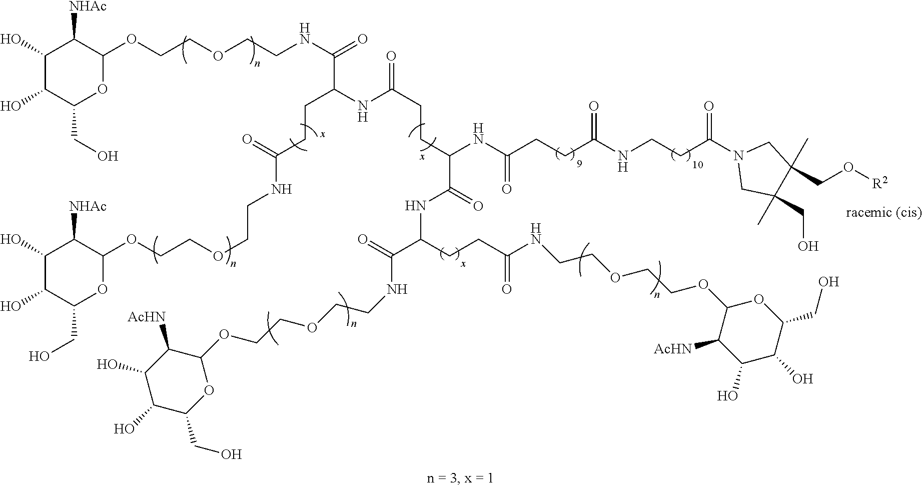

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

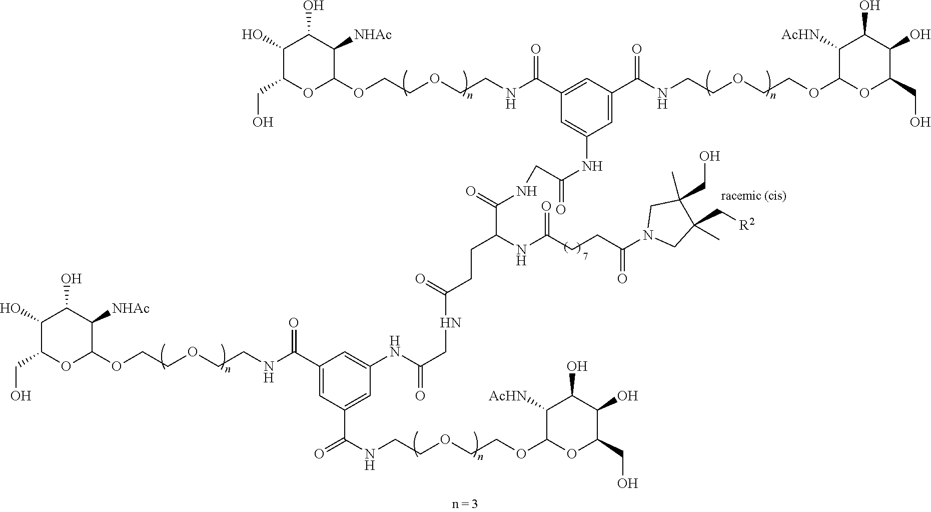

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

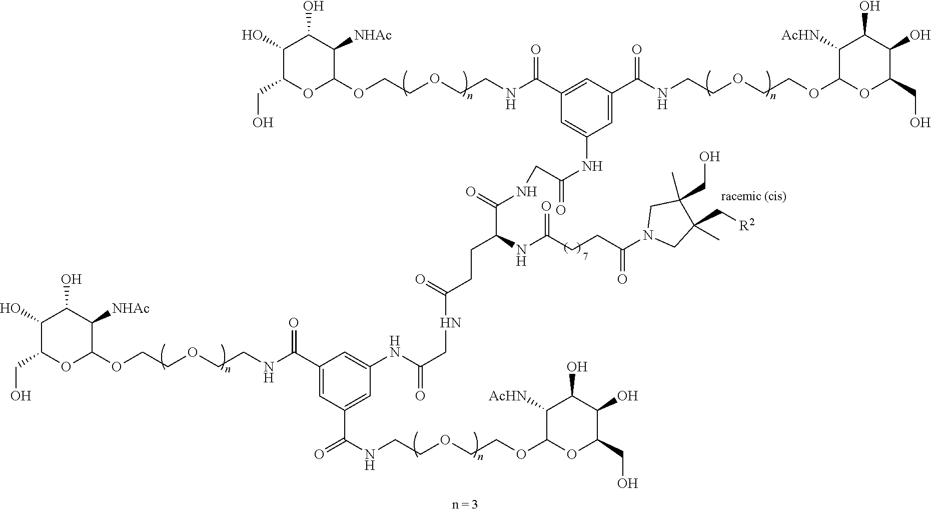

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

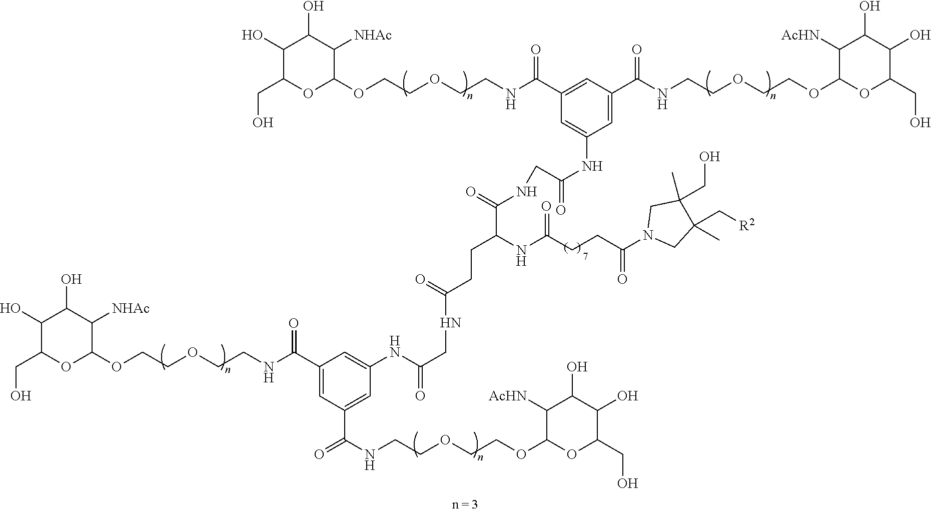

or a pharmaceutically acceptable salt thereof, wherein R² is a double stranded siRNA molecule (e.g. a double stranded siRNA molecule selected from the double stranded siRNA molecules disclosed herein, e.g., in FIG. 1).

In one embodiment the compound of formula I is:

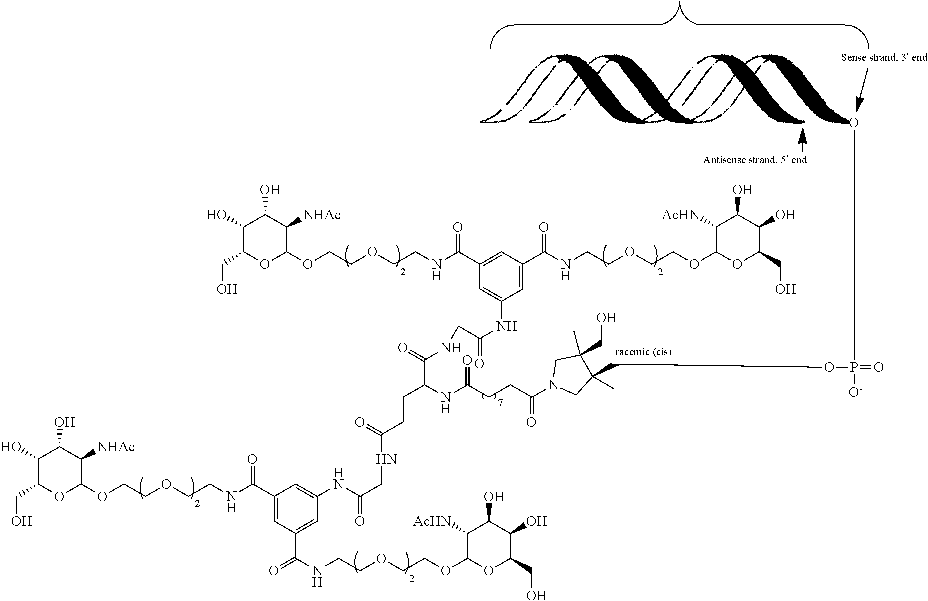

wherein the siRNA is selected from siRNA 1-siRNA 119, or a pharmaceutically acceptable salt thereof.

In one embodiment the compound of formula I is:

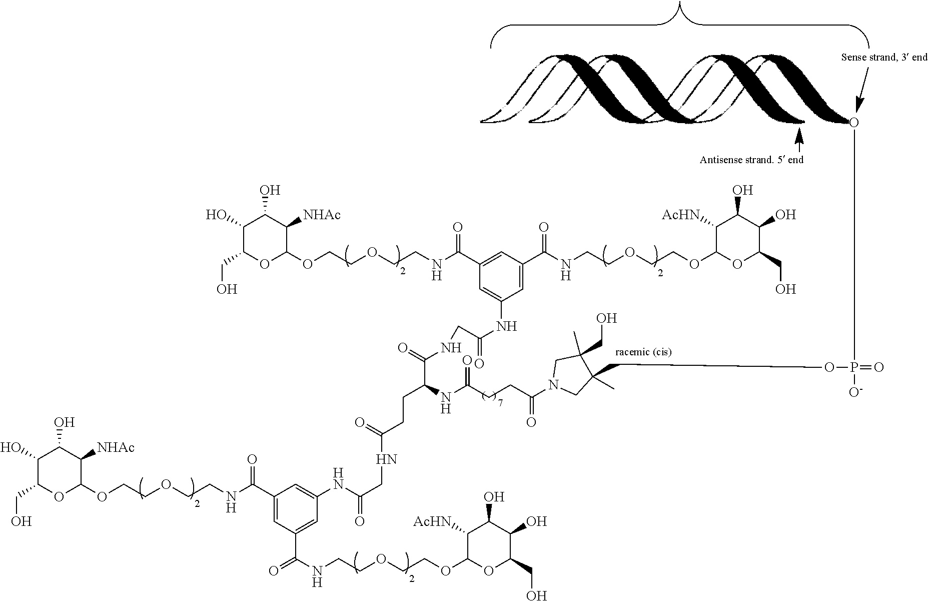

wherein the siRNA is selected from siRNA 1-siRNA 119, or a pharmaceutically acceptable salt thereof.

In one embodiment the compound of formula I is:

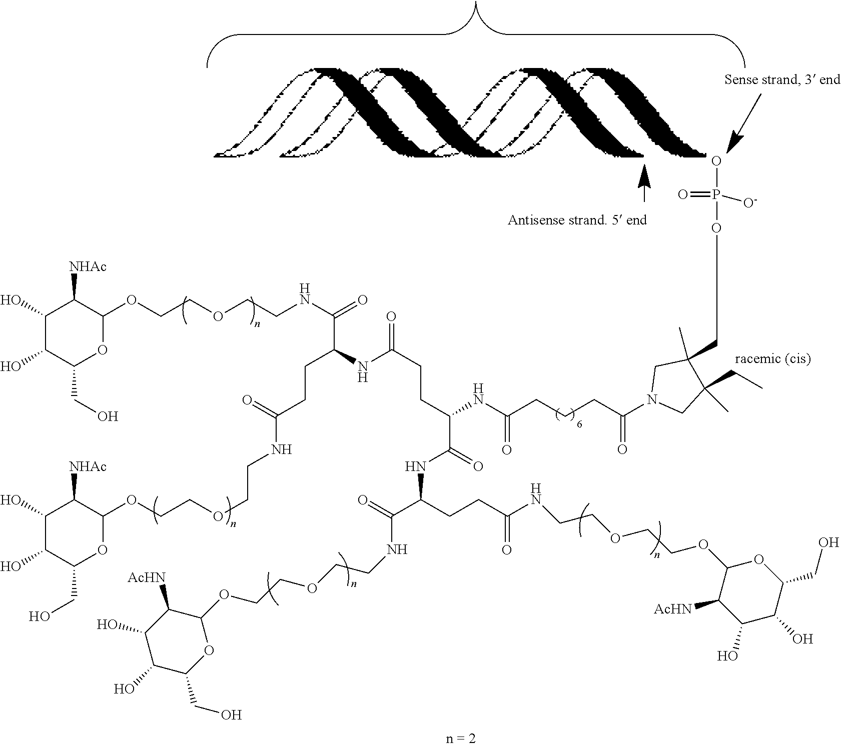

wherein the siRNA is selected from siRNA 1 -siRNA 119, or a pharmaceutically acceptable salt thereof.

In one embodiment the compound of formula I is:

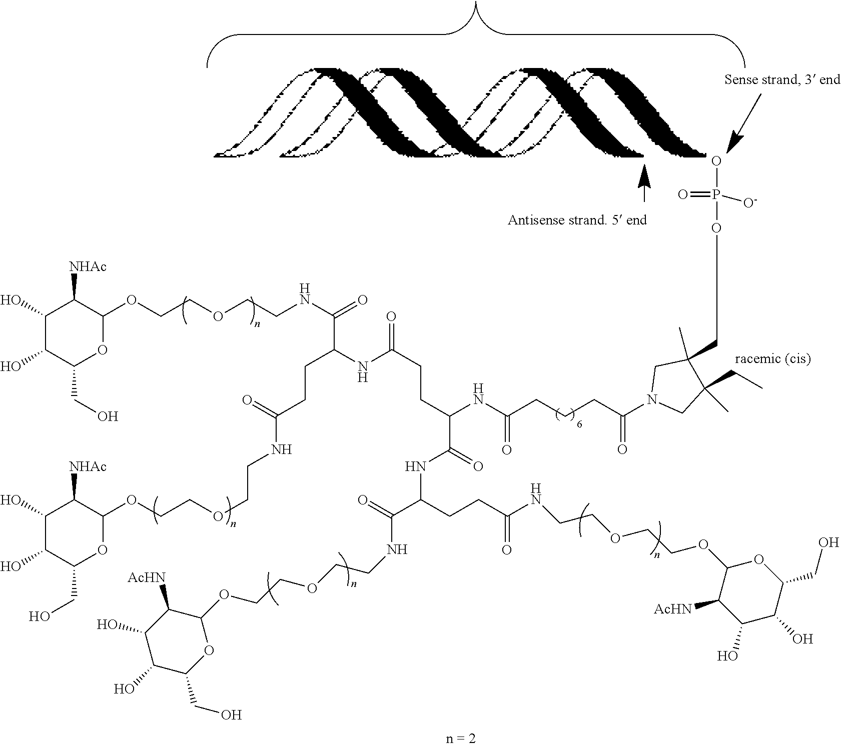

wherein the siRNA is selected from siRNA 1-siRNA 119, or a pharmaceutically acceptable salt thereof.

One embodiment provides a compound of formula (I):

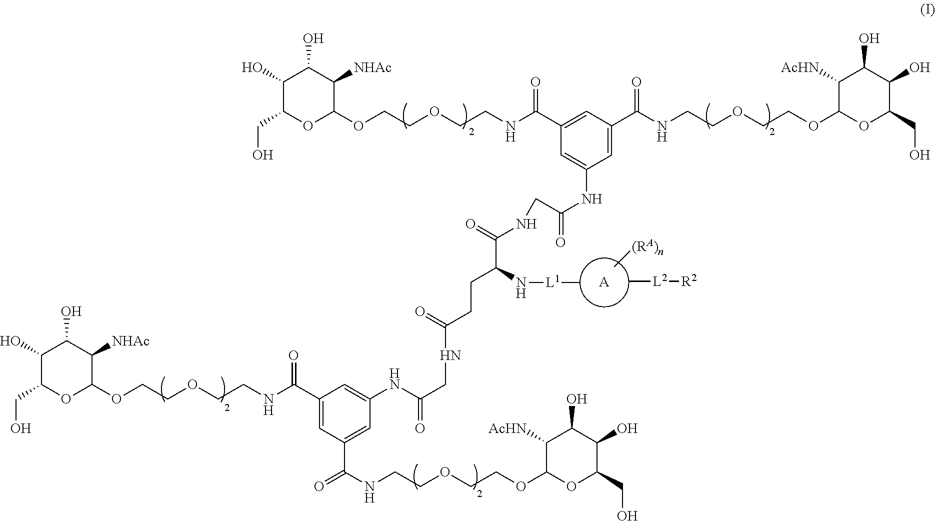

wherein:

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂alkyl-OR^{B}, C₁₋₁₀alkyl C₂₋₁₀alkenyl, and C₂₋₁₀alkynyl; wherein the C₁₋₁₀alkyl C₂₋₁₀alkenyl, and C₂₋₁₀alkynyl are optionally substituted with one or more groups independently selected from halo, hydroxy, and C₁₋₃alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

One embodiment provides a compound of formula:

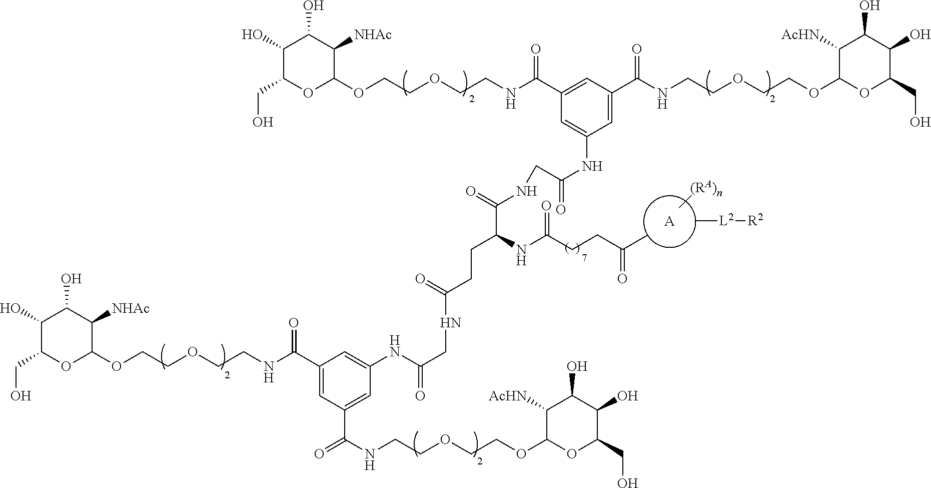

wherein:

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂alkyl-OR^{B}, C₁₋₁₀alkyl C₂₋₁₀alkenyl, and C₂₋₁₀alkynyl; wherein the C₁₋₁₀alkyl C₂₋₁₀alkenyl, and C₂₋₁₀alkynyl are optionally substituted with one or more groups independently selected from halo, hydroxy, and C₁₋₃alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

One embodiment provides a compound of formula:

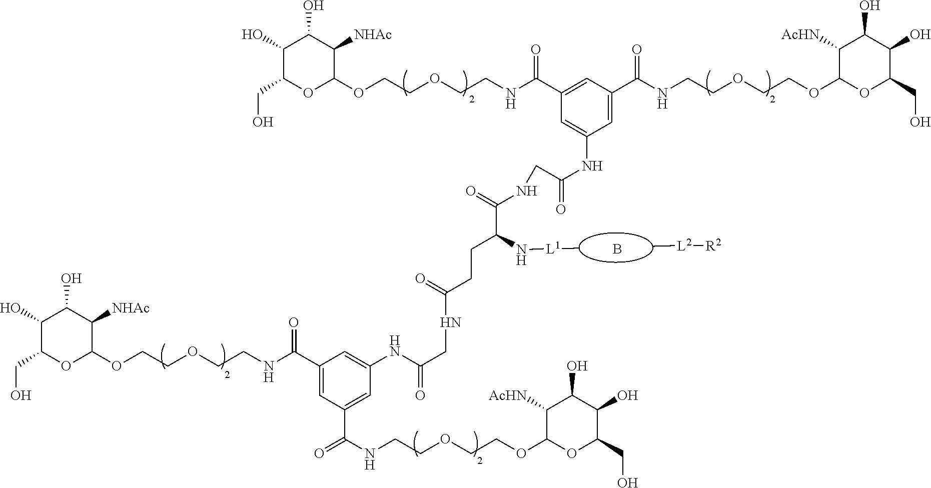

wherein:

L¹ is absent or a linking group;

L² is absent or a linking group;

R² is a nucleic acid;

B is divalent and is selected from the group consisting of:

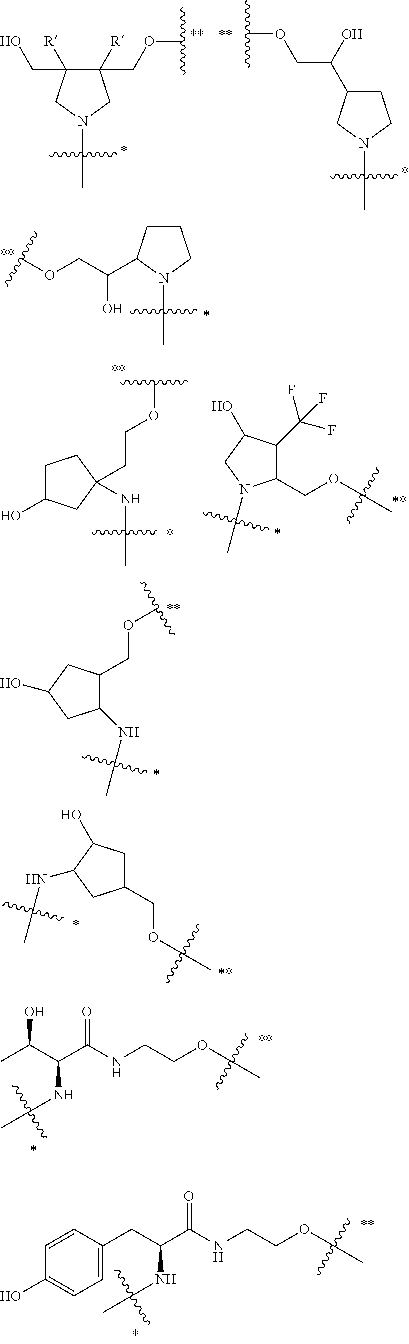

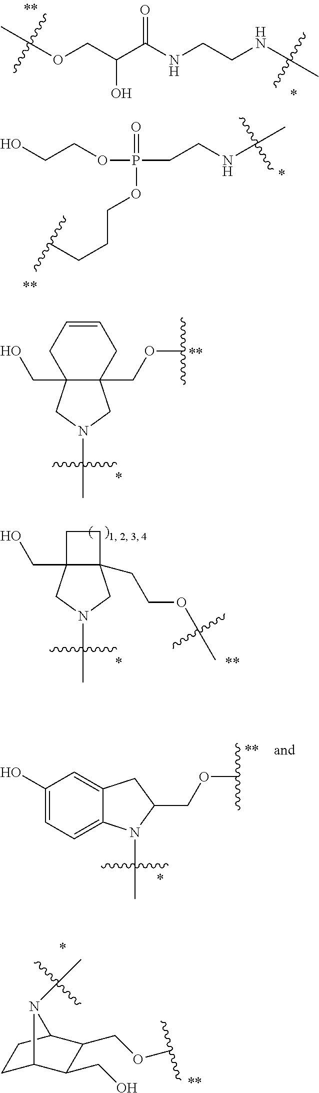

wherein:

each R′ is independently C₁₋₉alkyl, C₂₋₉alkenyl or C₂₋₉alkynyl; wherein the C₁₋₉alkyl, C₂₋₉alkenyl or C₂₋₉alkynyl are optionally substituted with halo or hydroxyl;

the valence marked with * is attached to L¹ or is attached to R¹ if L¹ is absent; and

the valence marked with ** is attached to L² or is attached to R² if L² is absent;

or a salt thereof.

In one embodiment L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from (C₁-C₆)alkoxy, (C₃-C₆)cycloalkyl, (C₁-C₆)alkanoyl, (C₁-C₆)alkanoyloxy, (C1-C6)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ is selected from the group consisting of:

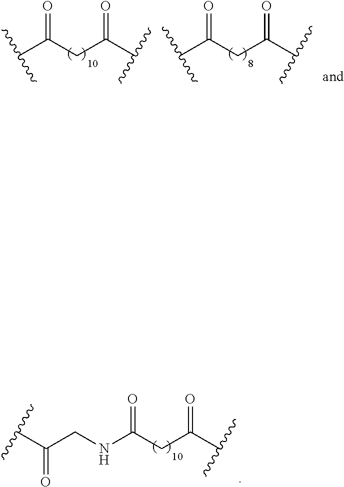

or a salt thereof.

In one embodiment L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In one embodiment L¹ is selected from the group consisting of:

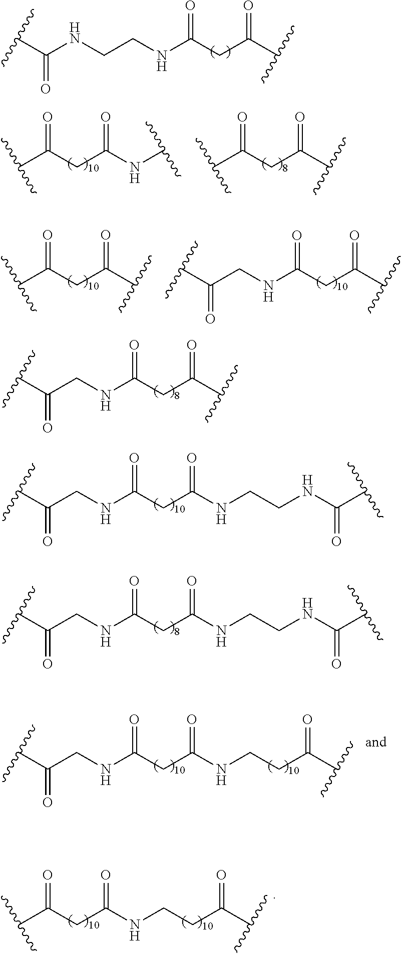

In one embodiment L₂ is connected to R₂ through

In one embodiment L² is C₁₋₄alkylene-O— that is optionally substituted with hydroxy.

In one embodiment L² is absent.

One embodiment provides a compound,

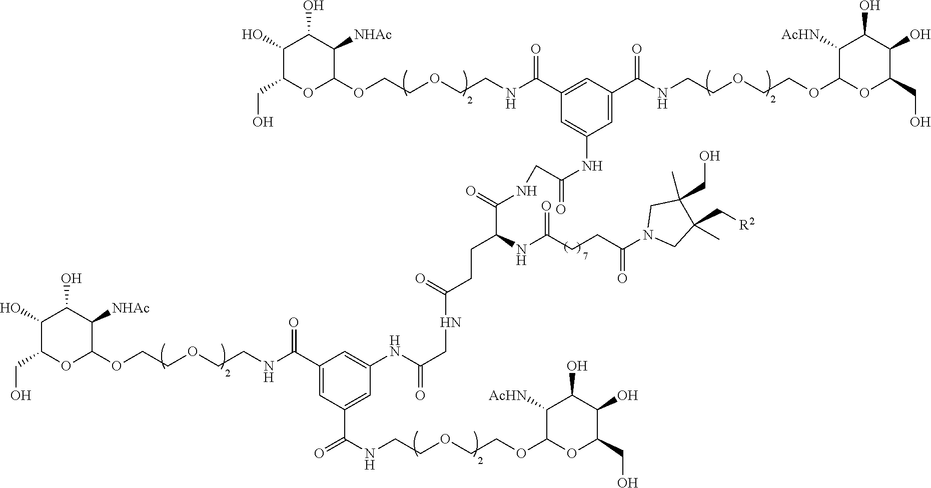

or a salt thereof wherein R² is a nucleic acid.

One aspect is pharmaceutical composition comprising a compound of formula I, and a pharmaceutically acceptable carrier.

Another aspect is a method to deliver a double stranded siRNA to the liver of an animal comprising administering a compound of formula I or a pharmaceutically acceptable salt thereof, to the animal.

Another aspect is a method to treat a disease or disorder in an animal comprising administering a compound of formula I or a pharmaceutically acceptable salt thereof, to the animal.

Certain embodiments provide a compound of formula (I) or a pharmaceutically acceptable salt thereof for use in medical therapy.

Certain embodiments provide a compound of formula (I) or a pharmaceutically acceptable salt thereof for the prophylactic or therapeutic treatment of a disease or disorder in an animal.

Certain embodiments provide the use of a compound of formula (I) or a pharmaceutically acceptable salt thereof to prepare a medicament for treating a disease or disorder in an animal.

In certain embodiments, the animal is a mammal, such as a human.

In one embodiment a compound of formula I has the following formula (Id):

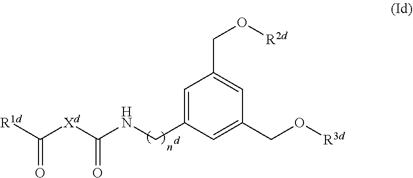

wherein:

R^{1d} is selected from:

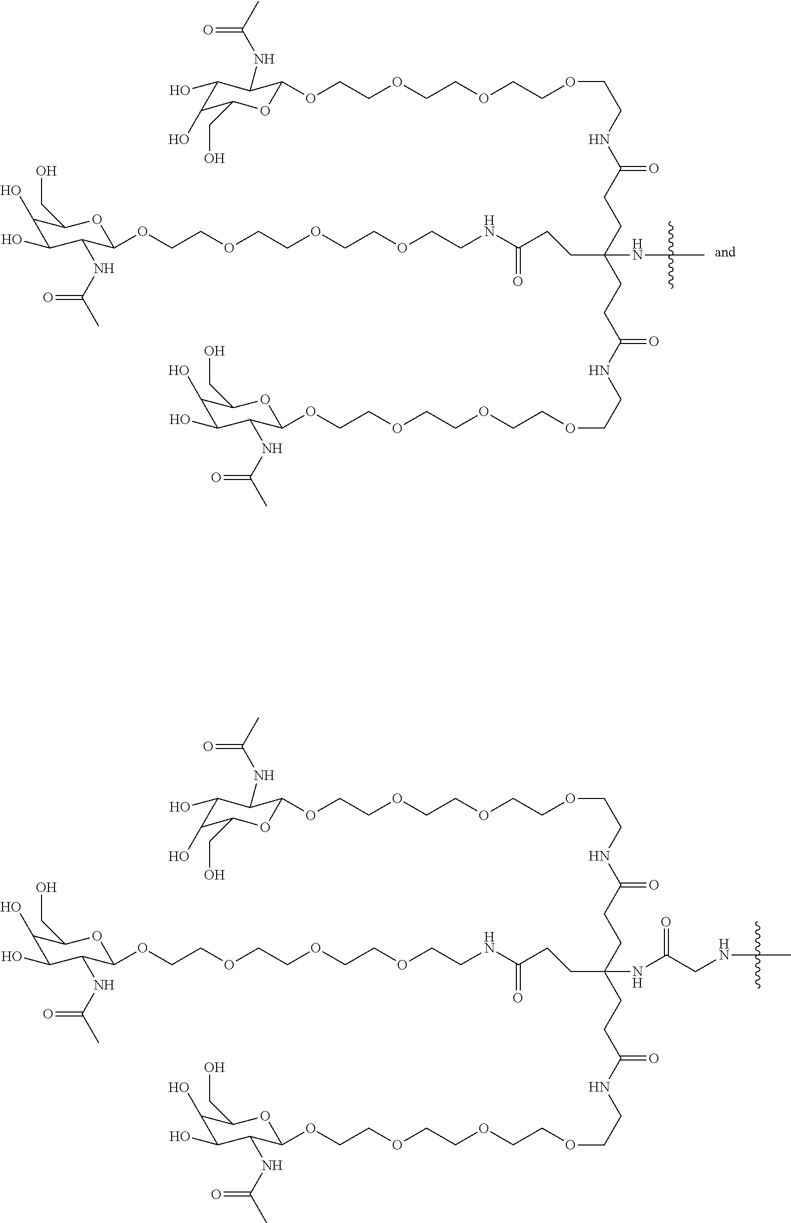 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₁₀alkylene;

n^{d} is 0 or 1;

R^{2d} is a double stranded siRNA molecule selected from the double stranded siRNA disclosed herein, e.g., in FIG. 1; and

R^{3d} is H, a protecting group, a covalent bond to a solid support, or a bond to a linking group that is bound to a solid support.

In one embodiment R^{3d} includes a linking group that joins the remainder of the compound of formula Id to a solid support. The nature of the linking group is not critical provided the compound is a suitable intermediate for preparing a compound of formula Id wherein R^{2d} is a double stranded siRNA molecule selected from the double stranded siRNA disclosed herein, e.g., in FIG. 1.

In one embodiment the linker in R^{3d} has a molecular weight of from about 20 daltons to about 1,000 daltons.

In one embodiment the linker in R^{3d} has a molecular weight of from about 20 daltons to about 500 daltons.

In one embodiment the linker in R^{3d} separates the solid support from the remainder of the compound of formula I by about 5 angstroms to about 40 angstroms, inclusive, in length.

In one embodiment the linker in R^{3d} is a divalent, branched or unbranched, saturated or unsaturated, hydrocarbon chain, having from 2 to 15 carbon atoms, wherein one or more (e.g.

1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment the linker in R^{3d} is a divalent, branched or unbranched, saturated or unsaturated, hydrocarbon chain, having from 2 to 10 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment the linker in R^{3d} is —C(═O)CH₂CH₂C(═O)N(H)—.

In one embodiment R^{1d} is:

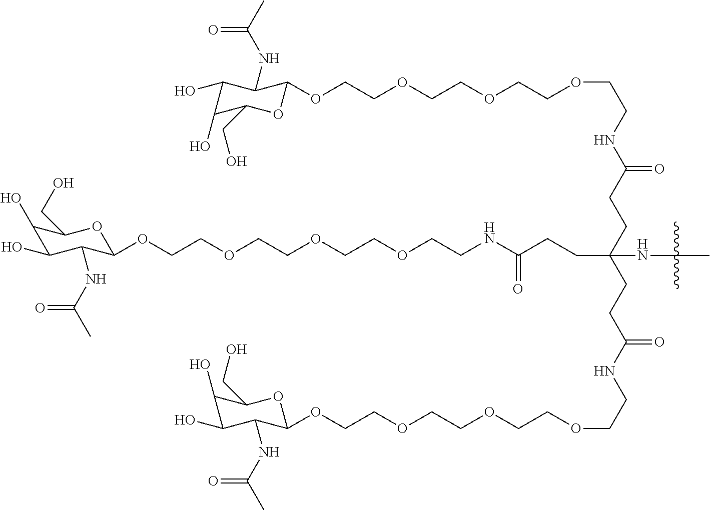 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In one embodiment R^{1d} is:

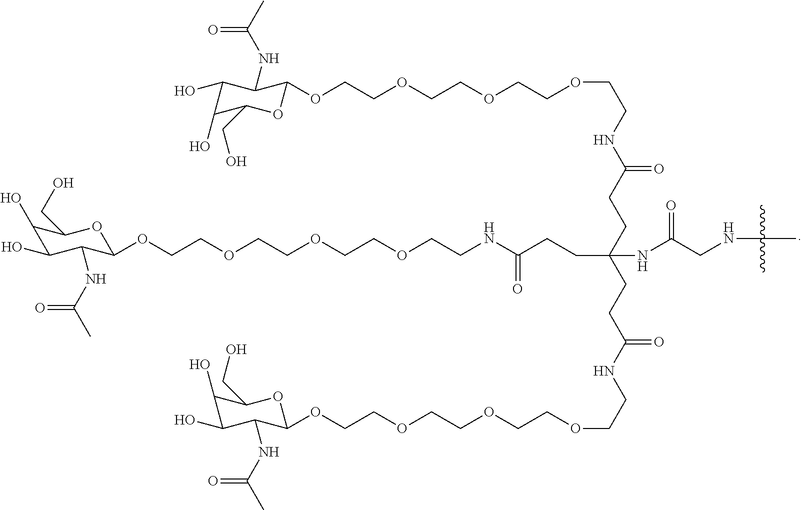 [SMILES: *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In one embodiment X^{d} is C₈alkylene.

In one embodiment n^{d} is 0.

In one embodiment R^{2d} is an siRNA.

In one embodiment R^{3d} is H.

In another embodiment a compound of (Id) or the salt thereof is selected from the group consisting of:

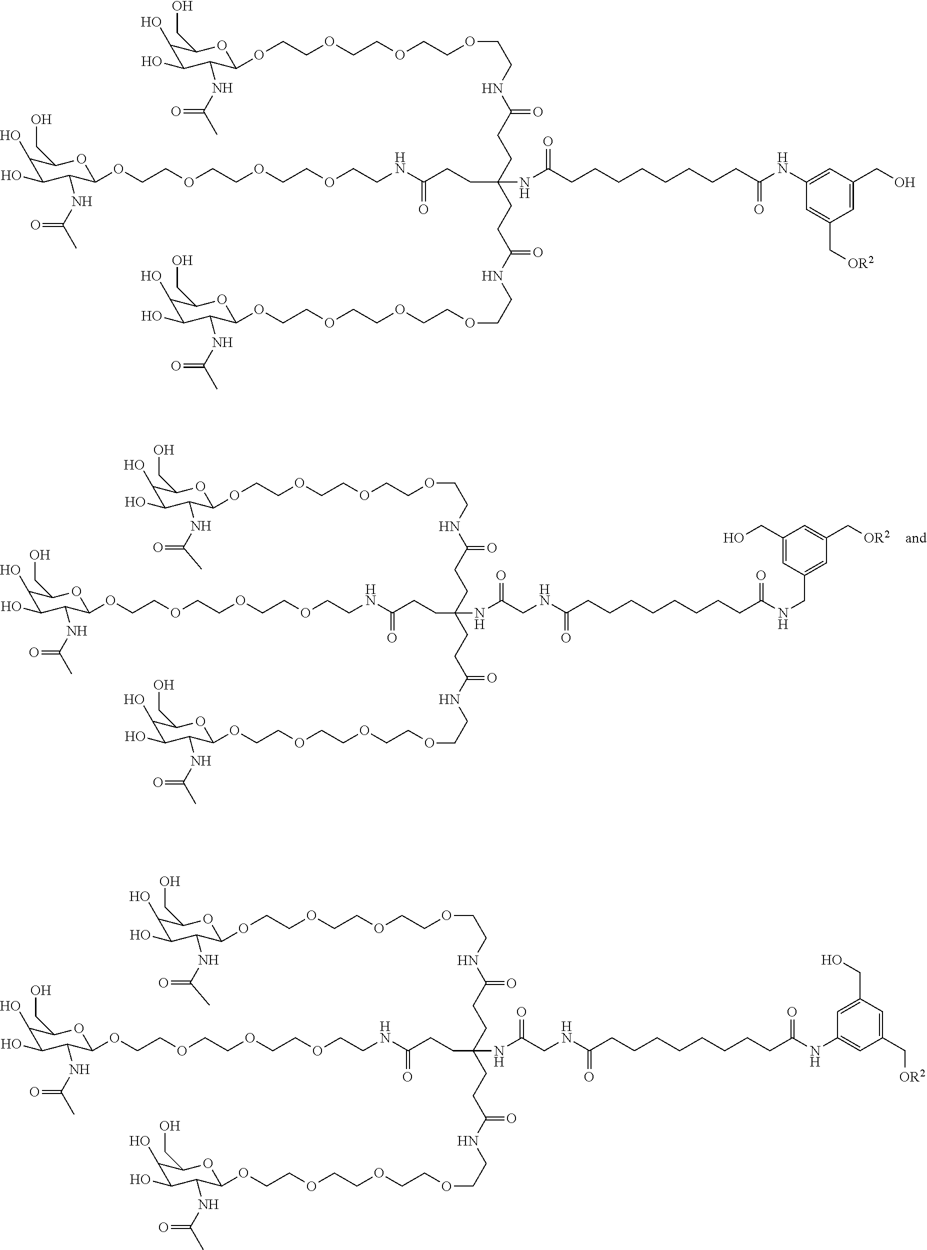

and salts thereof.

One aspect is a pharmaceutical composition comprising a compound of formula (Id), and a pharmaceutically acceptable carrier.

One aspect is a method to deliver is a double stranded siRNA to the liver of an animal comprising administering a compound of formula (Id) or a pharmaceutically acceptable salt thereof, to the animal.

Another aspect is a method to treat a disease or disorder in an animal comprising administering a compound of formula (Id) or a pharmaceutically acceptable salt thereof, to the animal.

Certain embodiments provide a compound of formula (Id) or a pharmaceutically acceptable salt thereof for use in medical therapy.

Certain embodiments provide a compound of formula (Id) or a pharmaceutically acceptable salt thereof for the prophylactic or therapeutic treatment of a disease or disorder in an animal.

Certain embodiments provide the use of a compound of formula (Id) or a pharmaceutically acceptable salt thereof to prepare a medicament for treating a disease or disorder in an animal.

In certain embodiments, the animal is a mammal, such as a human.

Also provided is a method to prepare a compound of formula (Id) as described herein comprising subjecting a corresponding compound of formula (Ie):

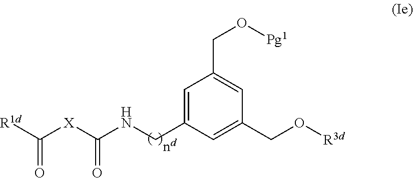

wherein:

X^{d} is C₂₋₈alkylene;

n^{d} or 1;

Pg¹ is H; and

R^{3d} is a covalent bond to a solid support or a bond to a linking group that is bound to a solid support, to solid phase nucleic acid synthesis conditions to provide a corresponding compound of formula Id wherein R^{2d} is a double stranded siRNA molecule selected from the double stranded siRNA molecules disclosed herein, e.g., in FIG. 1.

In one embodiment the method further comprises removing the compound from the solid support to provide the corresponding compound of formula Id wherein R^{3d} is H.

In one embodiment the compound is not a compound formula Ie:

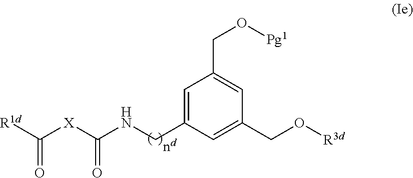

or a salt thereof, wherein:

R^{1d} is selected from:

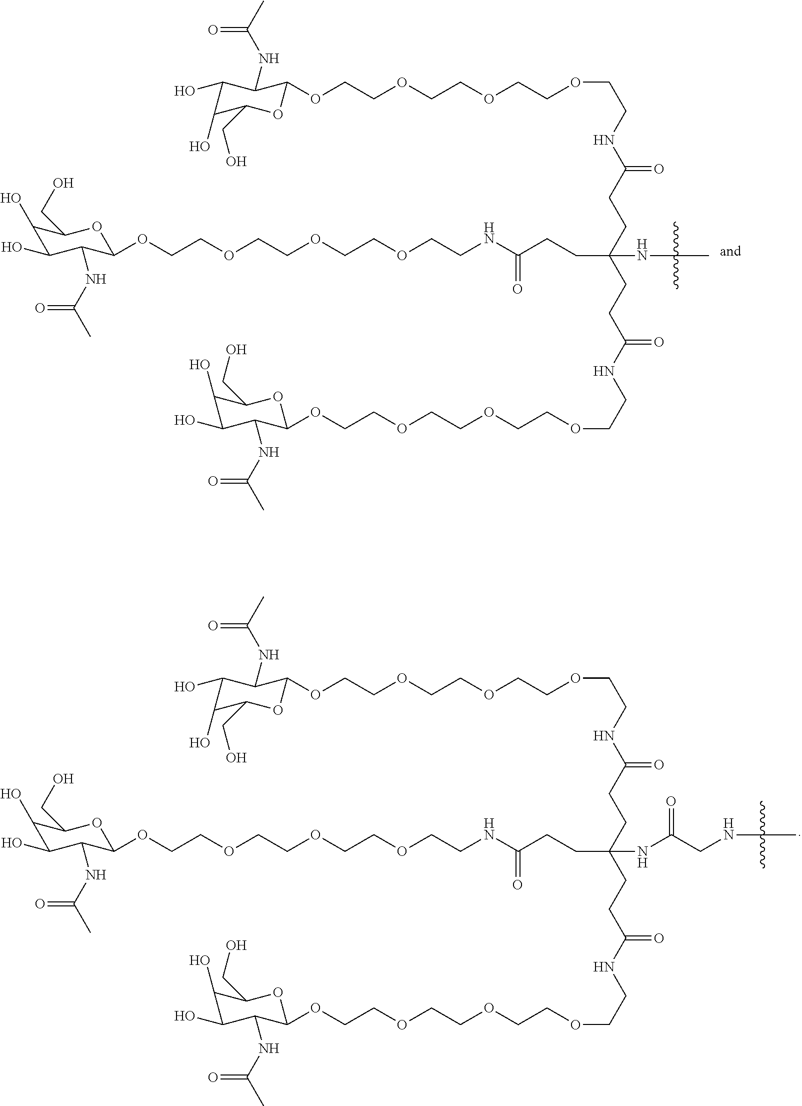 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

x^{d} is C₂₋₈alkylene;

n^{d} is 0 or 1;

Pg¹ is H or a suitable protecting group; and

R^{3d} is H, a protecting group, a covalent bond to a solid support, or a bond to a linking group that is bound to a solid support.

In one embodiment R^{3d} is H.

In one embodiment R^{3d} is a covalent bond to a solid support.

In one embodiment R^{3d} is a bond to a linking group that is bound to a solid support, wherein the linking group is a divalent, branched or unbranched, saturated or unsaturated, hydrocarbon chain, having from 2 to 15 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment R^{3d} is a bond to a linking group that is bound to a solid support, wherein the linking group is a divalent, branched or unbranched, saturated or unsaturated, hydrocarbon chain, having from 2 to 10 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment R^{3d} is a bond to a linking group that is bound to a solid support, wherein the linking group is —C(═O)CH₂CH2C(═O)N(H)—.

One embodiment provides a compound of formula (I):

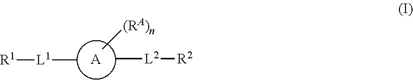

wherein:

R¹ is H or a synthetic activating group;

L¹ is absent or a linking group;

L² is absent or a linking group;

R² is a double stranded siRNA molecule selected from the double stranded siRNA molecules disclosed herein, e.g., in FIG. 1;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂alkyl-OR^{B}, C₁₋₁₀alkyl C₂₋₁₀alkenyl, and C₂₋₁₀alkynyl; wherein the C₁-₁₀alkyl C₂₋₁₀alkenyl, and C₂₋₁₀alkynyl are optionally substituted with one or more groups independently selected from halo, hydroxy, and C₁₋₃alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

One embodiment provides a compound of formula (Ig):

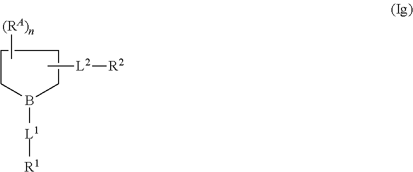

wherein:

B is —N— or —CH—;

L² is C₁₋₄alkylene-O— that is optionally substituted with hydroxyl or halo; and

n is 0, 1, 2, 3, 4, 5, 6, or 7;

or a salt thereof.

One embodiment provides a compound selected from the group consisting of:

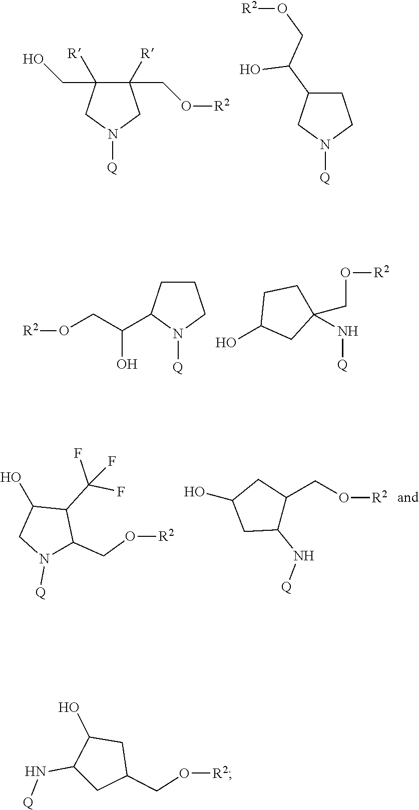

wherein:

Q is -L¹-R¹; and

R′ is C1-9alkyl, C₂₋₉alkenyl or C₂₋₉alkynyl; wherein the C₁₋₉alkyl, C₂₋₉alkenyl or C₂₋₉ alkynyl are optionally substituted with halo or hydroxyl;

and salts thereof.

One embodiment provides a compound selected from the group consisting of:

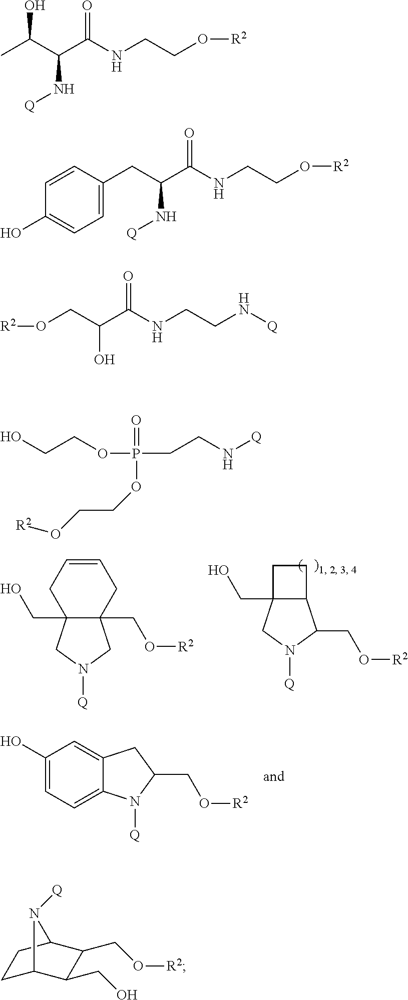

wherein: Q is —L¹-R¹; and salts thereof.

In one embodiment L¹ is a divalent, branched or unbranched, saturated or unsaturated, hydrocarbon chain, having from 5 to 20 carbon atoms, wherein one or more (e.g. 1, 2, 3, or 4) of the carbon atoms in the hydrocarbon chain is optionally replaced —O—, —NH—, —NH—C(═O)—, —C(═O)—NH— or —S—.

One embodiment provides a compound of formula (XX):

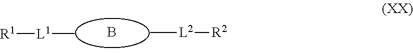

wherein:

R¹ a is targeting ligand;

L¹ is absent or a linking group;

L² is absent or a linking group;

R² is a double stranded siRNA molecule selected from the double stranded siRNA molecules disclosed herein, e.g., in FIG. 1;

B is divalent and is selected from the group consisting of:

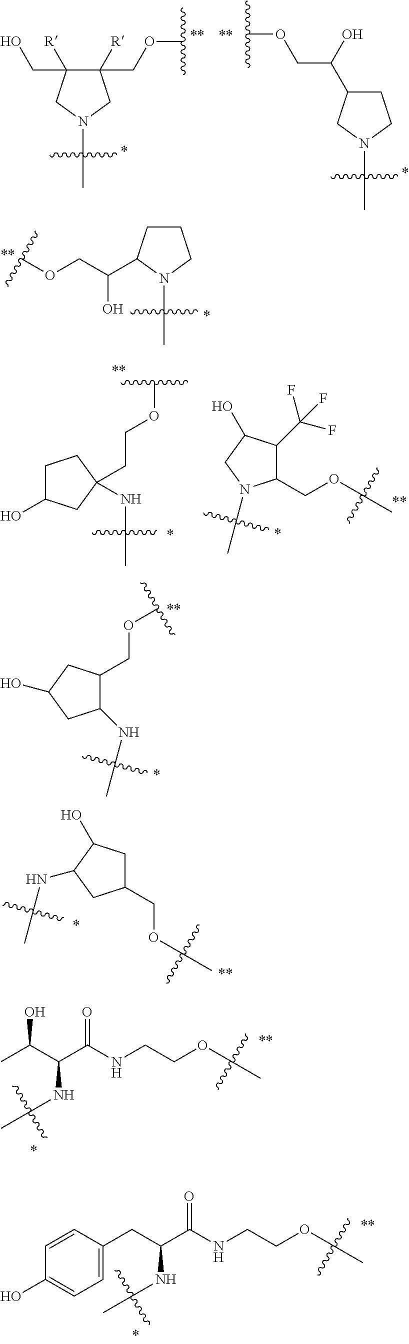

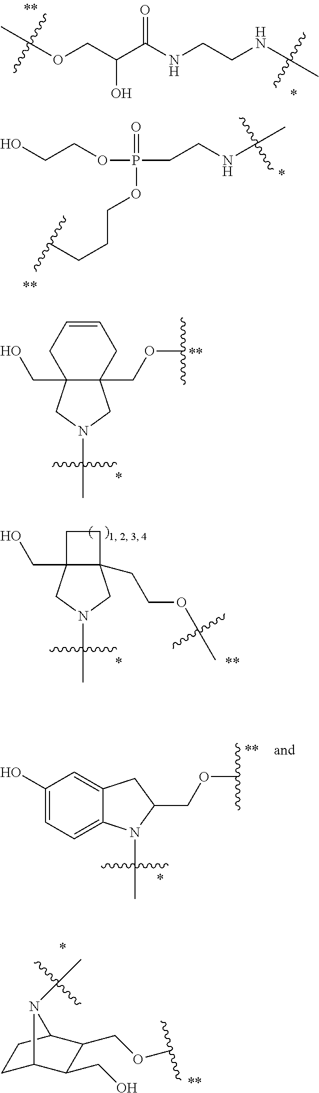

wherein:

each R¹ is independently C1-9alkyl, C₂₋₉alkenyl or C₂₋₉alkynyl; wherein the C₁₋₉alkyl, C₂₋₉alkenyl or C₂₋₉alkynyl are optionally substituted with halo or hydroxyl;

the valence marked with * is attached to L¹ or is attached to R¹ if L¹ is absent; and

the valence marked with ** is attached to L² or is attached to R² if L² is absent;

or a salt thereof.

In one embodiment R¹ comprises 2-8 saccharides.

In one embodiment R¹ comprises 2-6 saccharides.

In one embodiment R¹ comprises 2-4 saccharides.

In one embodiment R¹ comprises 3-8 saccharides.

In one embodiment R¹ comprises 3-6 saccharides.

In one embodiment R¹ comprises 3-4 saccharides.

In one embodiment R¹ comprises 3 saccharides.

In one embodiment R¹ comprises 4 saccharides.

In one embodiment R¹ has the following formula:

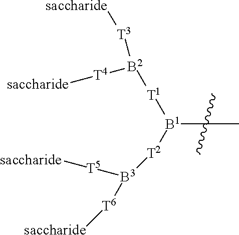

wherein:

B¹ is a trivalent group comprising about 1 to about 20 atoms and is covalently bonded to L¹, T¹, and T².

B² is a trivalent group comprising about 1 to about 20 atoms and is covalently bonded to T¹, T³, and T⁴;

B³ is a trivalent group comprising about 1 to about 20 atoms and is covalently bonded to T², T⁵, and T⁶; T¹ is absent or a linking group;

T² is absent or a linking group;

T³ is absent or a linking group;

T⁴ is absent or a linking group;

T⁵ is absent or a linking group; and

T⁶ is absent or a linking group

In one embodiment each saccharide is independently selected from:

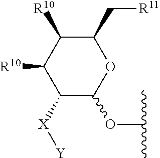

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In one embodiment each saccharide is independently selected from the group consisting of:

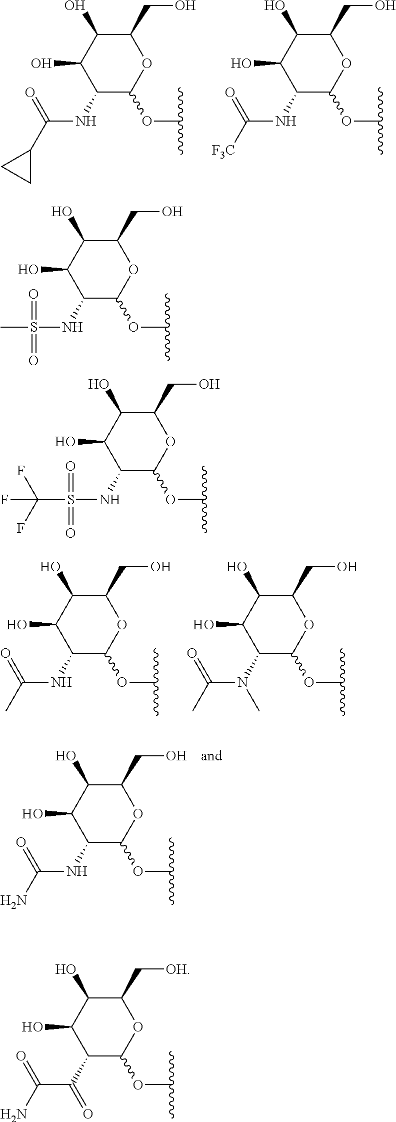

In one embodiment each saccharide is independently:

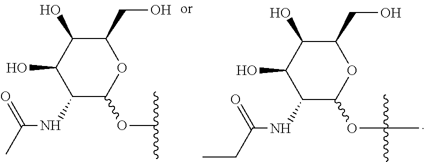 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment one of T¹ and T² is absent.

In one embodiment both T¹ and T² are absent.

In one embodiment each of T¹, T², T³, T⁴, T⁵, and T⁶ is independently absent or a branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C3-C6)cycloalkyl, (C₁-C₆)alkanoyl, (C₁-C₆)alkanoyloxy, (C₁-C₆)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment each of T¹, T², T³, T⁴, T⁵, and T⁶ is independently absent or a branched or unbranched, saturated or unsaturated, hydrocarbon chain, having from 1 to 50 carbon atoms, or a salt thereof, wherein one or more (e.g. 1, 2, 3, or 4) of the carbon atoms in the hydrocarbon chain is optionally replaced by —O— or —NR^{X}—, and wherein R^{X} is hydrogen or (C₁-C₆)alkyl, and wherein the hydrocarbon chain, is optionally substituted with one or more (e.g. 1, 2, 3, or 4) substituents selected from halo, hydroxy, and oxo (═O).

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) sub stituents selected from halo, hydroxy, and oxo (═O).

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In one embodiment at least one of T³, T⁴, T⁵, and T⁶ is:

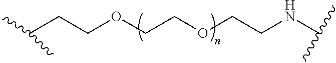

wherein:

n=1, 2, 3.

In one embodiment each of T³, T⁴, T⁵, and T⁶ is independently selected from the group consisting of:

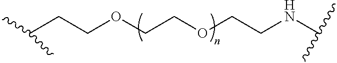

wherein:

n=1, 2, 3.

In one embodiment at least one of T¹ and T² is glycine

In one embodiment each of T¹ and T² is glycine.

In one embodiment B¹ is a trivalent group comprising 1 to 15 atoms and is covalently bonded to L¹, T¹, and T².

In one embodiment B¹ is a trivalent group comprising 1 to 10 atoms and is covalently bonded to L¹, T¹, and T².

In one embodiment B¹ comprises a (C₁-C₆)alkyl.

In one embodiment B¹ comprises a C₃₋₈cycloalkyl.

In one embodiment B¹ comprises a silyl group.

In one embodiment B¹ comprises a D- or L-amino acid.

In one embodiment B¹ comprises a saccharide.

In one embodiment B¹ comprises a phosphate group.

In one embodiment B¹ comprises a phosphonate group.

In one embodiment B¹ comprises an aryl.

In one embodiment B¹ comprises a phenyl ring.

In one embodiment B¹ is a phenyl ring.

In one embodiment B¹ is CH.

In one embodiment B¹ comprises a heteroaryl.

In one embodiment B¹ is selected from the group consisting of:

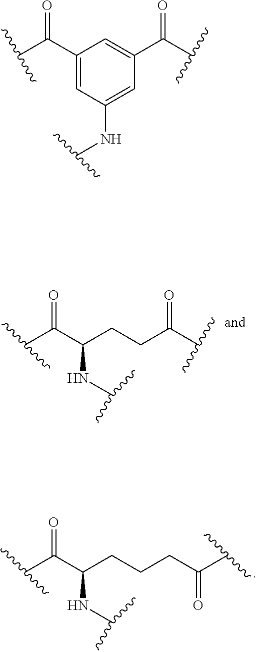 [SMILES: *Nc1cc(C(*)=O)cc(C(*)=O)c1 ; *N[C@H](CCC(*)=O)C(*)=O ; *N[C@H](CCCC(*)=O)C(*)=O]

In one embodiment B¹ is selected from the group consisting of:

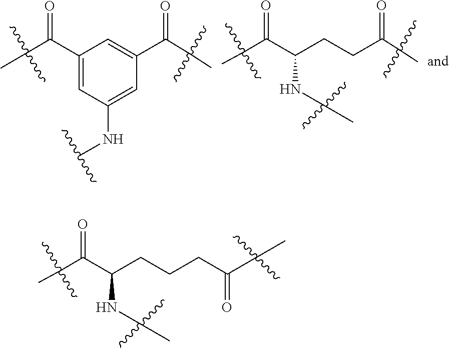 [SMILES: *Nc1cc(C(*)=O)cc(C(*)=O)c1 ; *N[C@@H](CCC(*)=O)C(*)=O ; *N[C@H](CCCC(*)=O)C(*)=O]

In one embodiment B² is a trivalent group comprising 1 to 15 atoms and is covalently bonded to L¹, T¹, and T².

In one embodiment B² is a trivalent group comprising 1 to 10 atoms and is covalently bonded to L¹, T¹, and T².

In one embodiment B² comprises a (C₁-C₆)alkyl

In one embodiment B² comprises a C₃₋₈cycloalkyl.

In one embodiment B² comprises a silyl group.

In one embodiment B² comprises a D- or L-amino acid.

In one embodiment B² comprises a saccharide.

In one embodiment B² comprises a phosphate group.

In one embodiment B² comprises a phosphonate group.

In one embodiment B² comprises an aryl.

In one embodiment B² comprises a phenyl ring.

In one embodiment B² is a phenyl ring.

In one embodiment B² is CH.

In one embodiment B² comprises a heteroaryl.

In one embodiment B² is selected from the group consisting of:

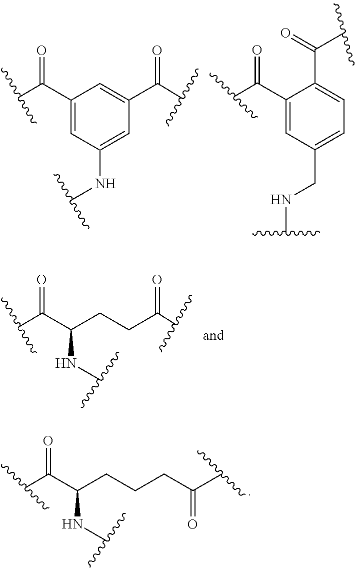 [SMILES: *Nc1cc(C(*)=O)cc(C(*)=O)c1 ; *NCc1ccc(C(*)=O)c(C(*)=O)c1 ; *N[C@H](CCC(*)=O)C(*)=O ; *N[C@H](CCCC(*)=O)C(*)=O]

In one embodiment B² is selected from the group consisting of:

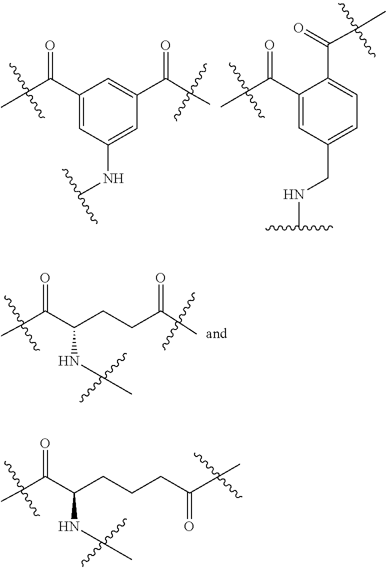 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

or a salt thereof.

In one embodiment B³ is a trivalent group comprising 1 to 15 atoms and is covalently bonded to L¹, T¹, and T².

In one embodiment B³ is a trivalent group comprising 1 to 10 atoms and is covalently bonded to L¹, T¹, and T².

In one embodiment B³ comprises a (C₁-C₆)alkyl.

In one embodiment B³ comprises a C₃₋₈cycloalkyl.

In one embodiment B³ comprises a silyl group.

In one embodiment B³ comprises a D- or L-amino acid.

In one embodiment B³ comprises a saccharide.

In one embodiment B³ comprises a phosphate group.

In one embodiment B³ comprises a phosphonate group.

In one embodiment B³ comprises an aryl.

In one embodiment B³ comprises a phenyl ring.

In one embodiment B³ is a phenyl ring.

In one embodiment B³ is CH.

In one embodiment B³ comprises a heteroaryl.

In one embodiment B³ is selected from the group consisting of:

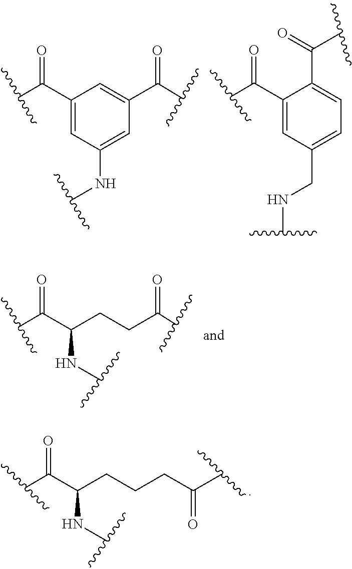 [SMILES: *Nc1cc(C(*)=O)cc(C(*)=O)c1 ; *NCc1ccc(C(*)=O)c(C(*)=O)c1 ; *N[C@H](CCC(*)=O)C(*)=O ; *N[C@H](CCCC(*)=O)C(*)=O]

In one embodiment B³ is selected from the group consisting of:

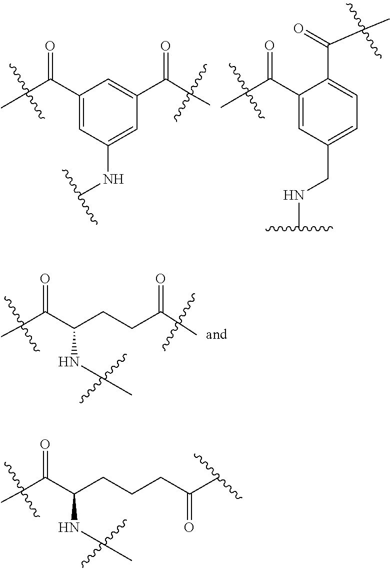 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

or a salt thereof.

In one embodiment L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment L¹ is selected from the group consisting of:

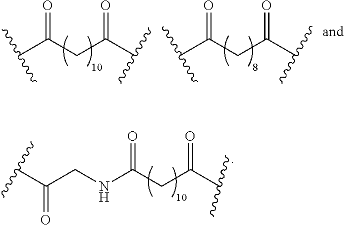

or a salt thereof.

In one embodiment L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In one embodiment L¹ is selected from the group consisting of:

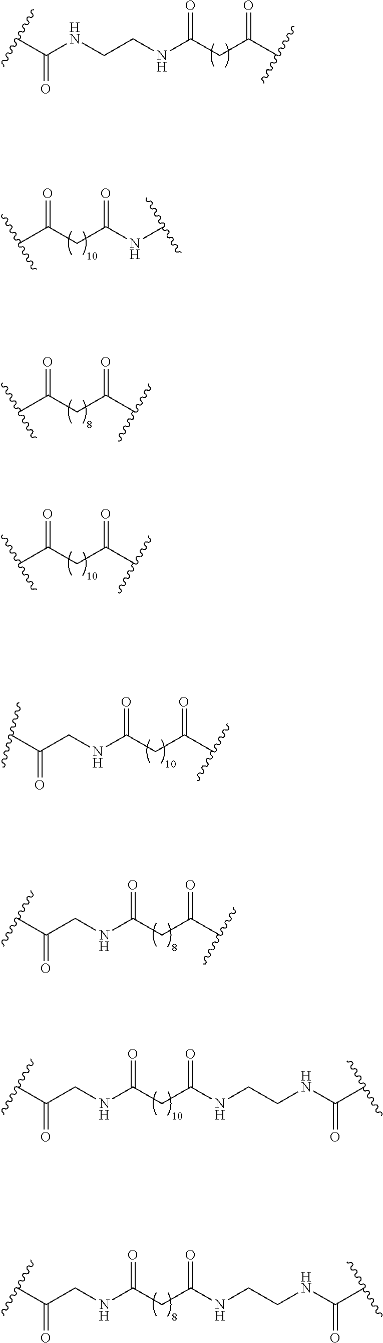

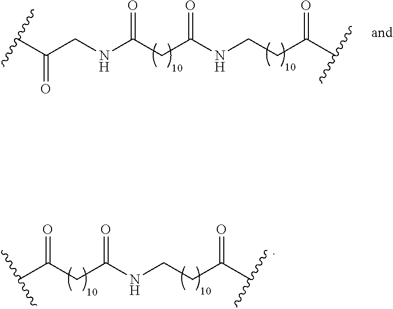

In one embodiment L² is connected to R² through —O—.

In one embodiment L² is C₁₋₄alkylene-O— that is optionally substituted with hydroxy.

In one embodiment L² is connected to R² through —O—.

In one embodiment L² is absent.

One embodiment provides a compound or salt selected from the group consisting of:

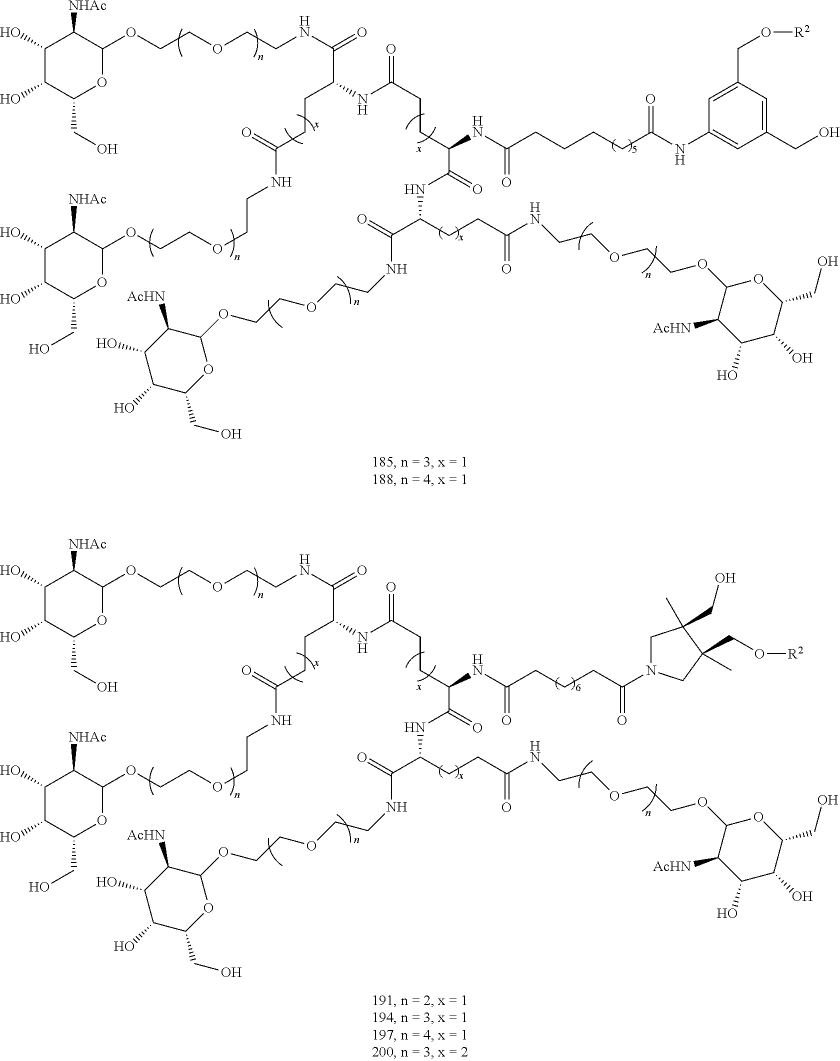

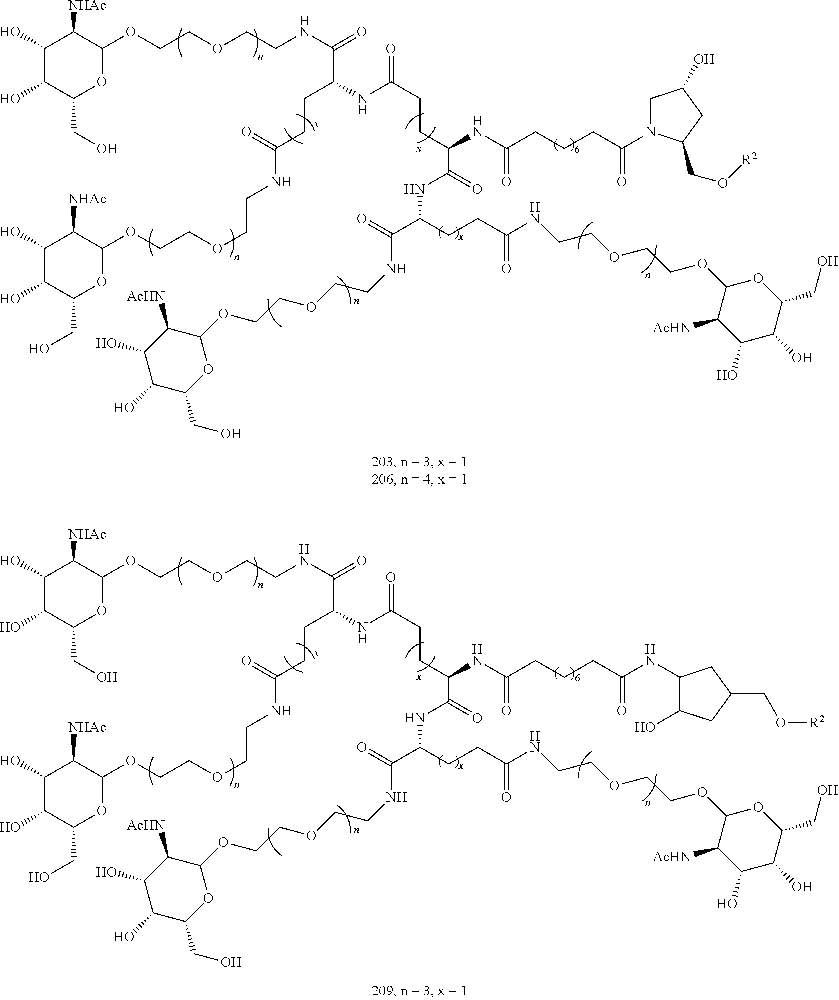

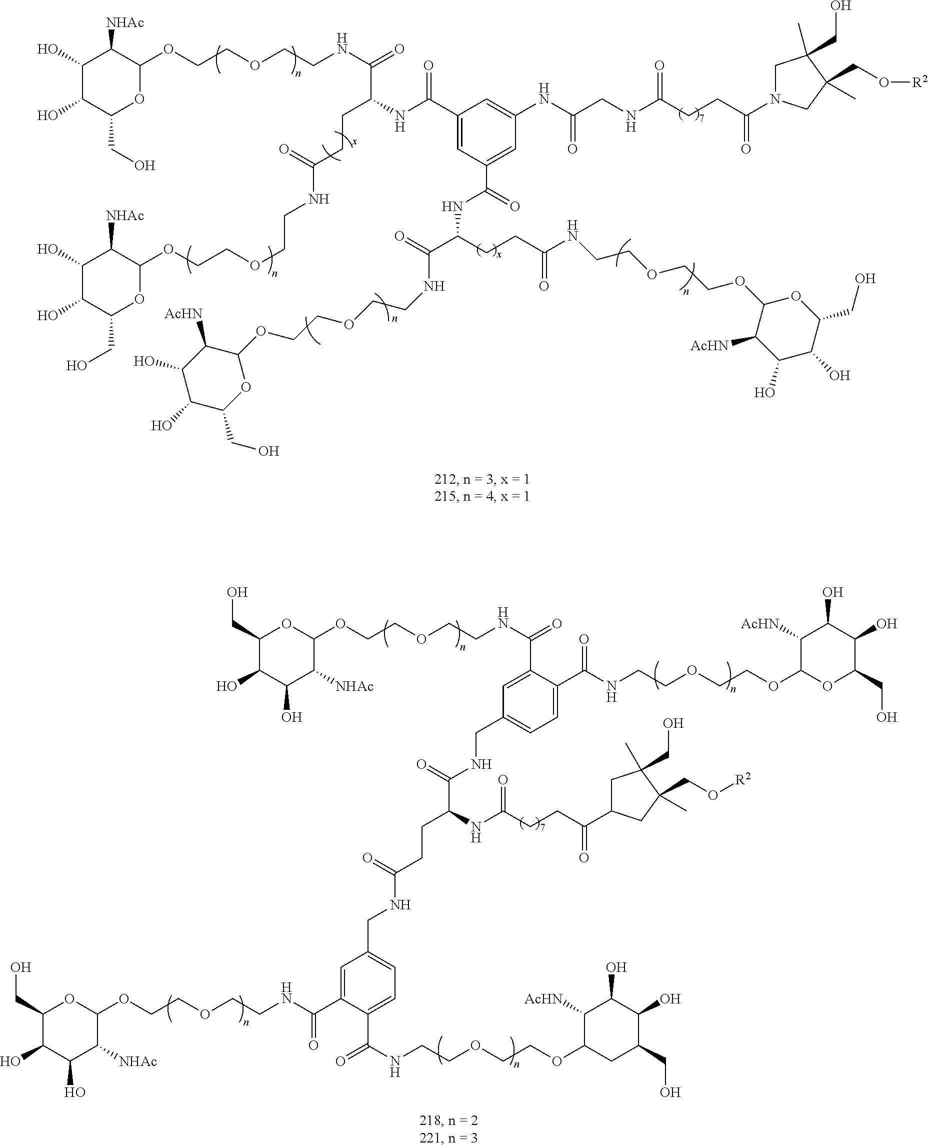

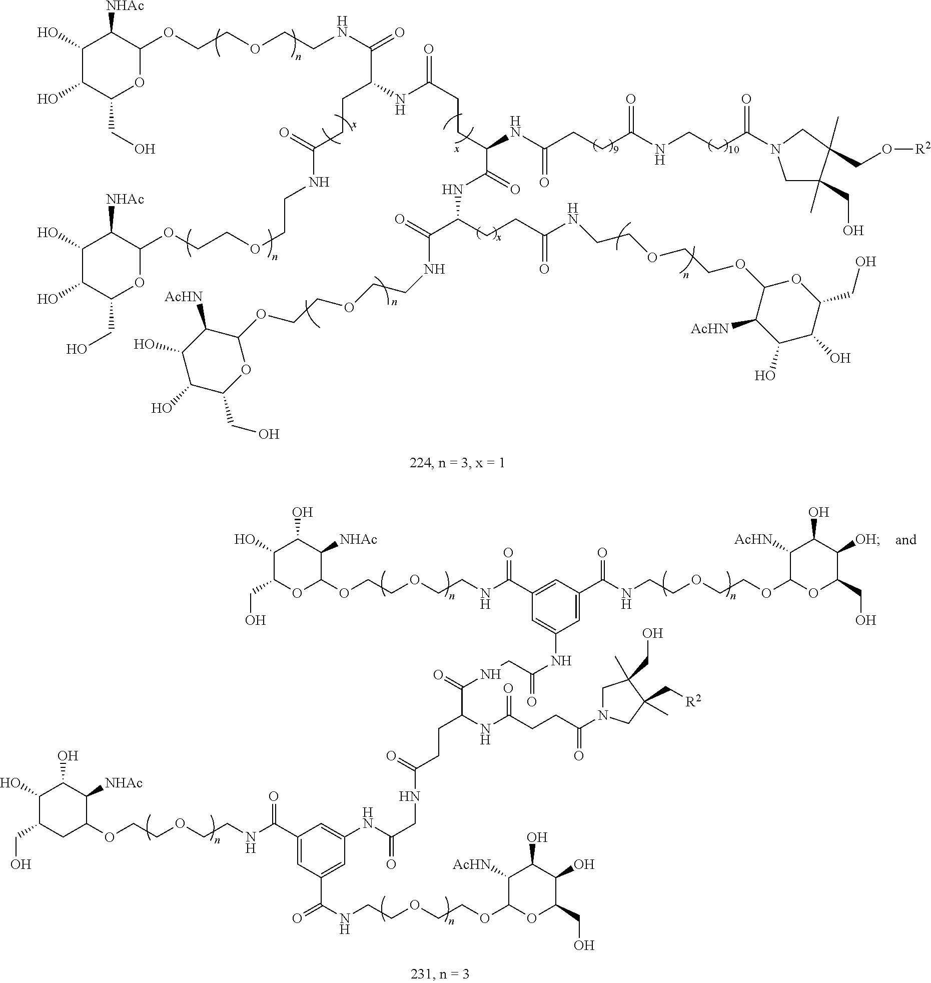

and pharmaceutically acceptable salts thereof, wherein R² is a double stranded siRNA molecule selected from the double stranded siRNA molecules disclosed herein, e.g., in FIG. 1.

One embodiment provides a compound of formula:

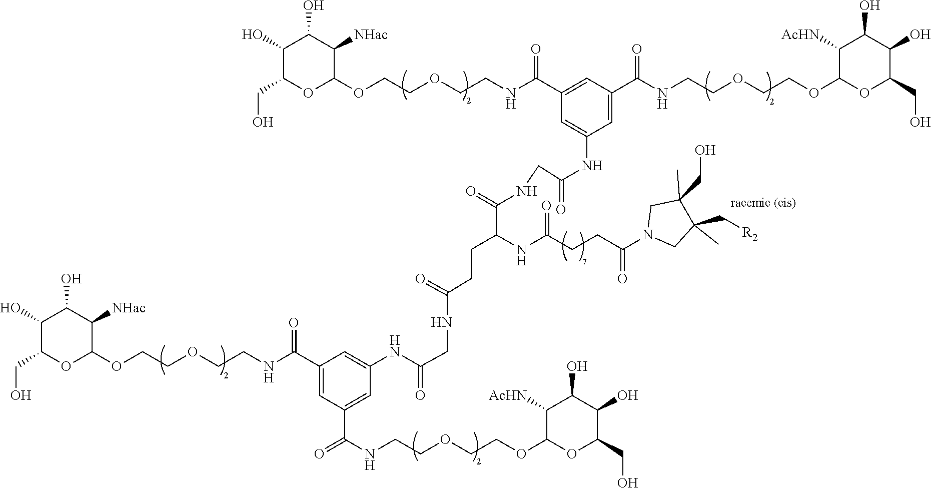

or a salt thereof wherein R² is a nucleic acid.

One embodiment provides a compound of formula:

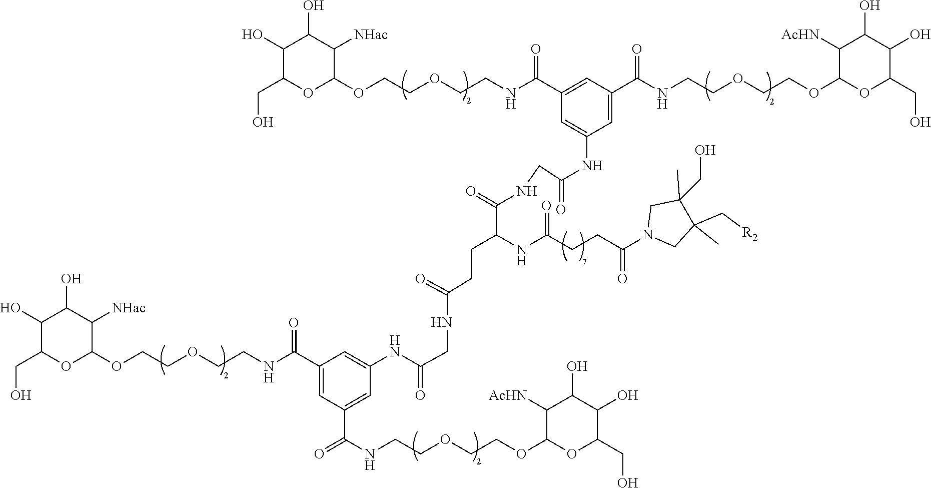

or a salt thereof wherein R² is a nucleic acid.

In one embodiment, the nucleic acid molecule (e.g., siRNA) is attached to the reminder of the compound through the oxygen of a phosphate at the 3′-end of the sense strand.

In one embodiment the compound or salt is administered subcutaneously.

When a compound comprises a group of the following formula:

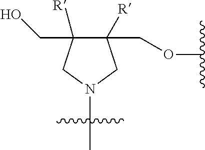

there are four stereoisomers possible on the ring, two cis and two trans. Unless otherwise noted, the compounds include all four stereoisomers about such a ring. In one embodiment, the two R′ groups are in a cis conformation. In one embodiment, the two R′ groups are in a trans conformation.

One aspect is a nucleic acid-lipid particle comprising:
(a) one or more double stranded siRNA molecules selected from the double stranded siRNA molecules disclosed herein, e.g., in FIG. 1;
(b) a cationic lipid; and
(c) a non-cationic lipid.

### Examples

The present invention will be described in greater detail by way of specific examples. The following examples are offered for illustrative purposes and are not intended to limit the invention in any manner. Those of skill in the art will readily recognize a variety of noncritical parameters which can be changed or modified to yield essentially the same results. It is understood that in one embodiment the oligonucleotide is a double stranded siRNA molecule as described in FIG. 1.

### Conjugates

As described herein, various conjugates can be used in the practice of the invention. For the Examples herein, the following conjugate was used. Additional conjuagtes useful with the siRNA molecules described herein are described in WO 2017/177326 (PCT/CA2017/050447) and in WO 2018/191278 (PCT/US2018/026918), the disclosures of which are each incorporated by reference.

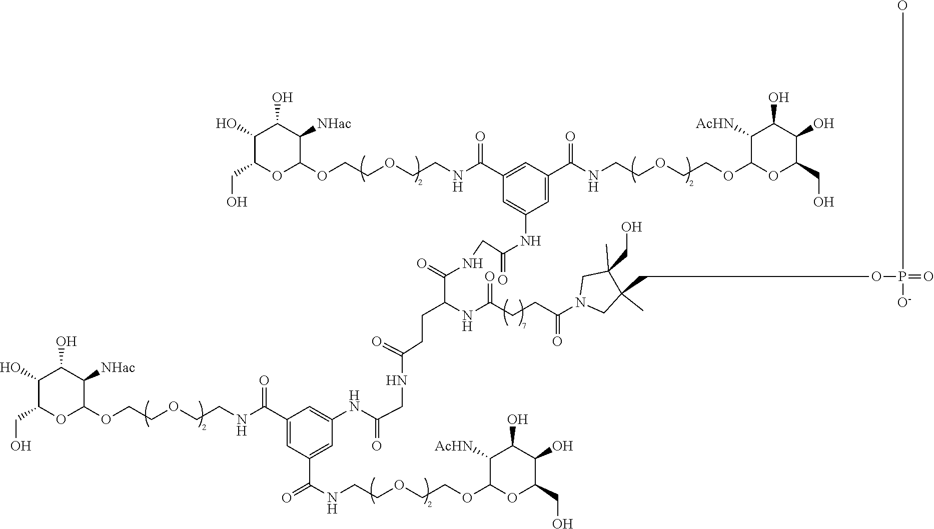

### siRNA Sequences

siRNA sequences used in the present Examples are depicted in FIG. 1.

### Example 1. A Pharmacodyamic Study in Non-Human Primates

### Objective

The objective of this study was to evaluate the pharmacodynamics of siRNA conjugates following a single administration via subcutaneous injection to male cynomolgus monkeys.

### Study Design

| | | | Dose | Dose | Dose | | Blood |
|---|---|---|---|---|---|---|---|
| Group | No. of | Test | Level | Concentration | Volume | Dose Route/ | Collections/Terminal |
| No. | Animals | Material | (mg/kg) | (mg/mL) | (mL/kg) | Regimen | Procedures |
| 1 | 4 M | Saline | 0 | 0 | 1 | Single SC dose on | Biomarkers: Day −14; |
| 2 | 4 M | siRNA 28 | 1 | 1 | | Day 1; 2 injection | Day 1 (predose) and |
| 3 | 4 M | | 3 | 3 | | sites with <= | Days 8, 15 |
| 4 | 4 M | siRNA 86 | 1 | 1 | | 2 mL/site; not | Clinical Pathology: |
| 5 | 4 M | | 3 | 3 | | fasted | Day 1 (predose), |
| 6 | 4 M | siRNA 59 | 1 | 1 | | | 8 and 15 |
| 7 | 4 M | | 3 | 3 | | | Terminal liver |
| | | | | | | | collection: Day 15 |

No. = Number;
M = Male;
SC = Subcutaneous

### Dose Formulation

The Test Article was be supplied in “ready to use” form (˜20% overage). The afternoon prior to dosing, the test articles were removed from the −80° C. storage and placed into 2-8° C. storage. On the day of dosing, the test articles were removed from the refrigerator and allowed to adjust to room temperature, at which point the samples were thoroughly mixed by gently inverting the vial a few times.

### Test System

Species: Macaca fascicularis

Strain: Cynomolgus macaque

Gender Male

Number of Males: 28

Age: Adult

Research Status: Naive

Weight: ˜1.6-2.3 kg

Source: Testing Facility Colony

Animals were fasted for 12 hours prior to all blood collection time points, except for the terminal timepoint. Filtered tap water was provided to experimental animals ad libitum via an automatic watering system throughout the duration of the experiment. It is considered that there are no known contaminants in the water that could interfere with the outcome of the study. Animals were euthanized on Day 15.

Dose levels (mg/kg) tested in this study were based on results from previous studies on these test articles, as well as other highly related oligonucleotide compounds in cynomolgus monkeys. In male Sprague Dawley rats, six subcutaneous doses of the three siRNA-conjugates used here (siRNAs 28, 86 and 59) were administered single dose at 20, 60 and 180 mg/kg. These treatments were generally well tolerated without clinical signs/adverse effects.

### Dose Administration

On the afternoon prior to dosing, the test articles were removed from the −80° C. storage and placed into 2-8° C. storage. On the day of dosing, the test articles w removed from the refrigerator and allowed to adjust to room temperature, at which point the samples were thoroughly mixed by gently inverting the vial a few times.

On Day 1, blood sampling was performed for all animals (same time and procedure as other time points) followed by dose administration. Animals were fed following blood collection and prior to dose administration (30-40 min period). Animals in Group 1 received a single administration via subcutaneous dose of 0.9% Sodium Chloride for Injection, USP; animals in Groups 2 and 3 received a single administration via subcutaneous dose of conjugate siRNA 28 at 1 and 3 mg/kg, respectively; animals in Groups 4 and 5 received a single administration via subcutaneous dose of conjugate siRNA 86 at 1 and 3 mg/kg, respectively; and animals in Groups 6 and 7 received a single administration via subcutaneous dose of conjugate siRNA 59 at 1 and 3 mg/kg, respectively.

All doses were administered in the intrascapular region. Each animal had a small area shaved on the back between the shoulder blades (scapular/dorsal region). The dosing area was wiped with alcohol and allowed to dry prior to each animal receiving multiple SC injections of conjugate administered at equal volumes at 2 dose sites on Dose Day 1. The dose volume for all injections was 1 mL/kg.

Whole Blood Collection and Processing for Clinical Chemistry

On Days 1 (predose) 8 and 15, whole blood samples 0.5 mL were collected from a peripheral vein via direct needle puncture into SST tubes and processed for serum according to Testing Facility SOP. Serum samples were stored at −80° C. until shipment for analysis.

Serum Chemistry Parameters

| | | |
|---|---|---|
| Alanine aminotransferase | Chloride | Potassium |
| Albumin | Cholesterol | Sodium |
| Alkaline Phosphatase | Creatinine Kinase | Total Bilirubin |
| Aspartate Aminotransferase | Creatinine | Total Protein |
| Bicarbonate | Gamma Glutamyl Transferase | Triglycerides |
| Bile Acids, Total | Glucose | |
| Bilirubin, Direct | High Density Lipoprotein | |
| Blood Urea Nitrogen | Low Density Lipoprotein | |
| BUN: Creatinine Ratio | Phosphorous | |

Data is presented as individual values by animal with means, standard deviation, as appropriate.

### Results

FIG. 4 depicts in vivo activity in cynomolgus monkes of certain siRNA conjugates of the invention. Single doses of GalNAc-siRNA conjugates were injected into male cynomolgus monkeys subcutaneously. At 14 days post injection, the liver from each animal was collected and the hepatic HSD17B13 mRNA level was determined by RT-qPCR and normalized to the average of 3 endogenous control mRNA levels (GAPDH, Arf1 and Eif1). The ratio of HSD17B13/endogenous control was further normalized to that of saline treated control animals. As depicted, at 14 days post-dose, one example of an siRNA conjugate reduced HSD17B13 mRNA levels significantly, with a 70% reduction at 3 mg/kg dose level.

What is claimed is:

1. A compound of formula (I): 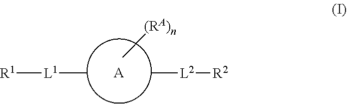
wherein:
R¹ a is targeting ligand;
L¹ is absent or a linking group;
L² is absent or a linking group;
R² is a siRNA molecule selected from any one of siRNA 1-siRNA 119;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂alkyl-OR^{B}, C₁₋₁₀alkyl C₂₋₁₀alkenyl, and C₂₋₁₀alkynyl; wherein the C₁₋₁₀alkyl C₂₋₁₀alkenyl, and C₂₋₁₀alkynyl are optionally substituted with one or more groups independently selected from halo, hydroxy, and C₁₋₃alkoxy;
R^{B} is hydrogen or a protecting group; and
n is 0, 1, 2, 3, 4, 5, 6, 7, 8, 9, or 10;
or a salt thereof.

2. The compound of claim 1, wherein R¹ is —C(H)₍₃₋ₚ₎(L³-saccharide)ₚ;
wherein each L³ is independently a linking group;
p is 1, 2, or 3; and
saccharide is a monosaccharide or disaccharide
or a salt thereof.

3. The compound of claim 2, wherein the saccharide is: 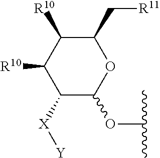
wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy;
or a salt thereof.

4. The compound of claim 2 or 3, wherein the saccharide is selected from the group consisting of: 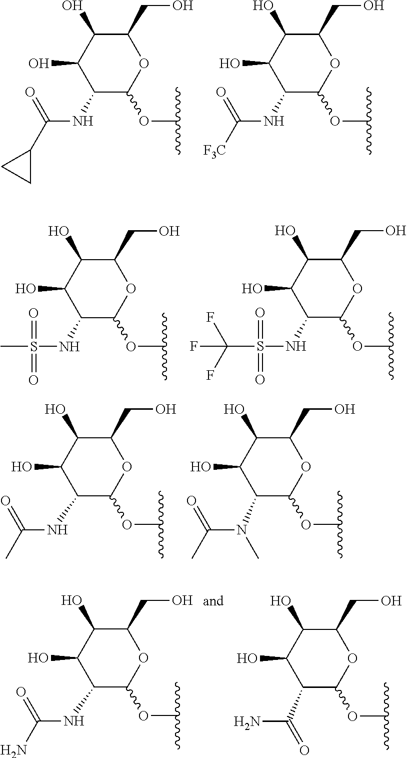
or a salt thereof.

5. The compound of any one of claims 2-4, wherein the saccharide is: 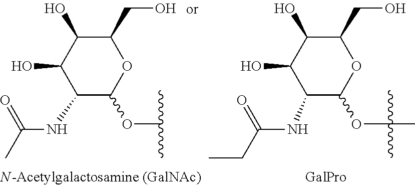 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]
or a salt thereof.

6. The compound of claim 1, wherein the compound of formula I is selected from the group consisting of: 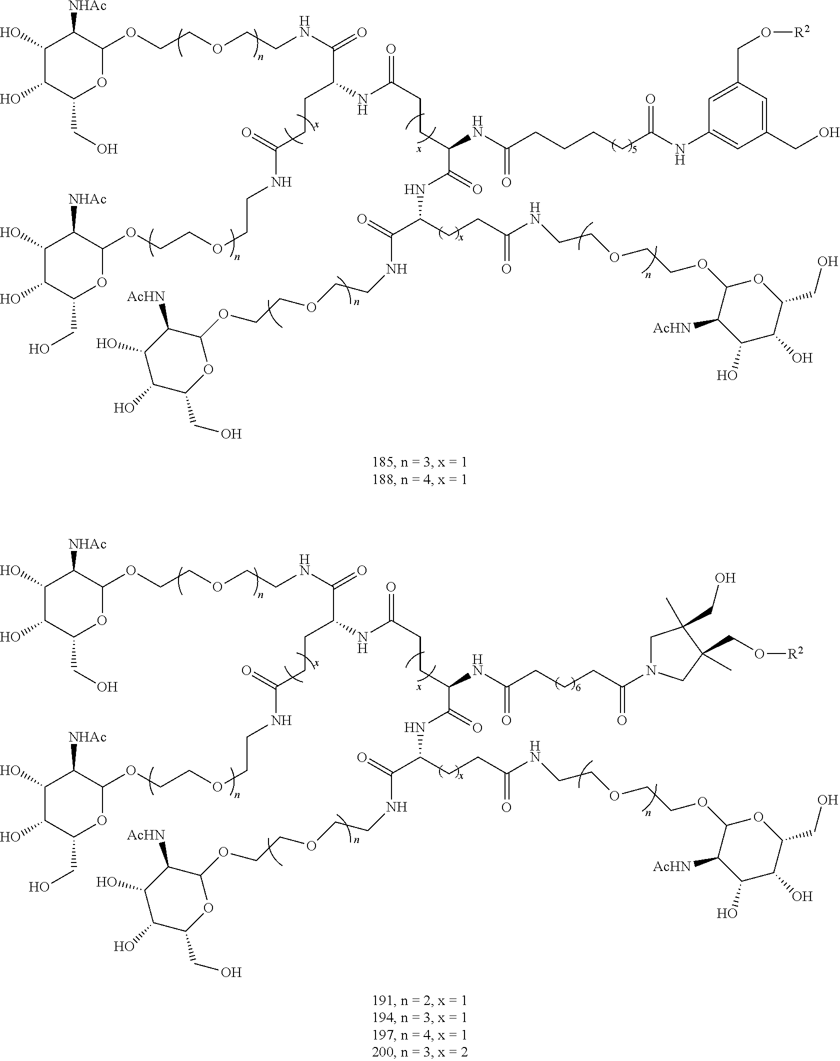 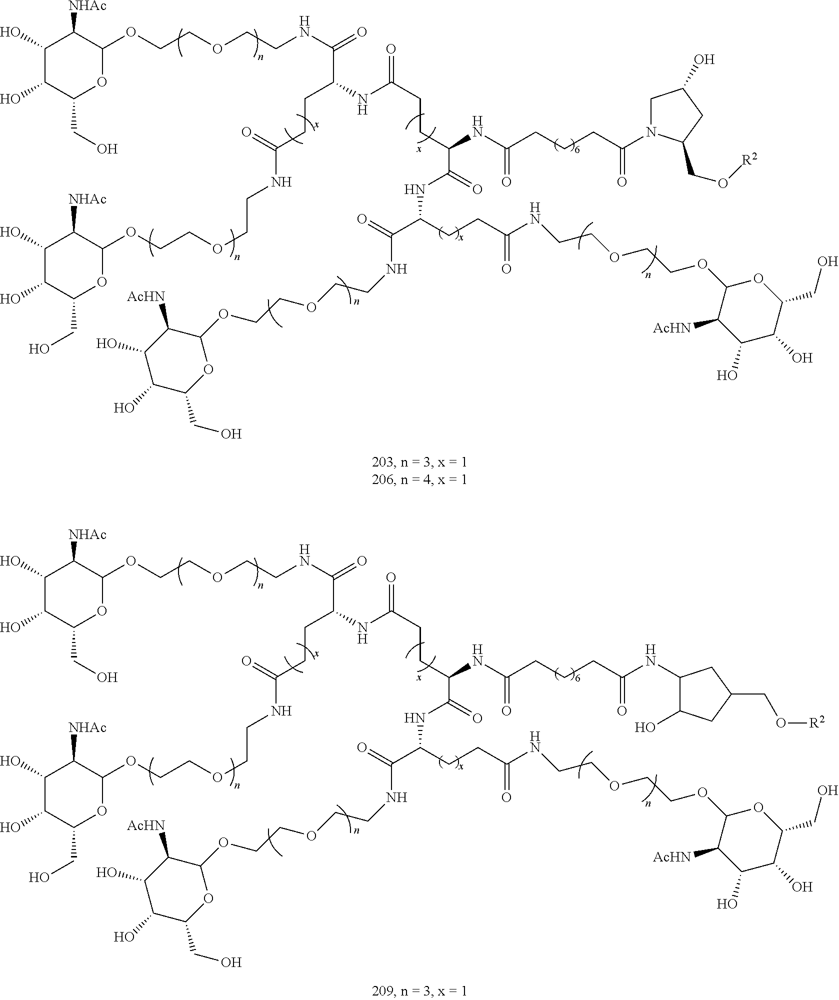 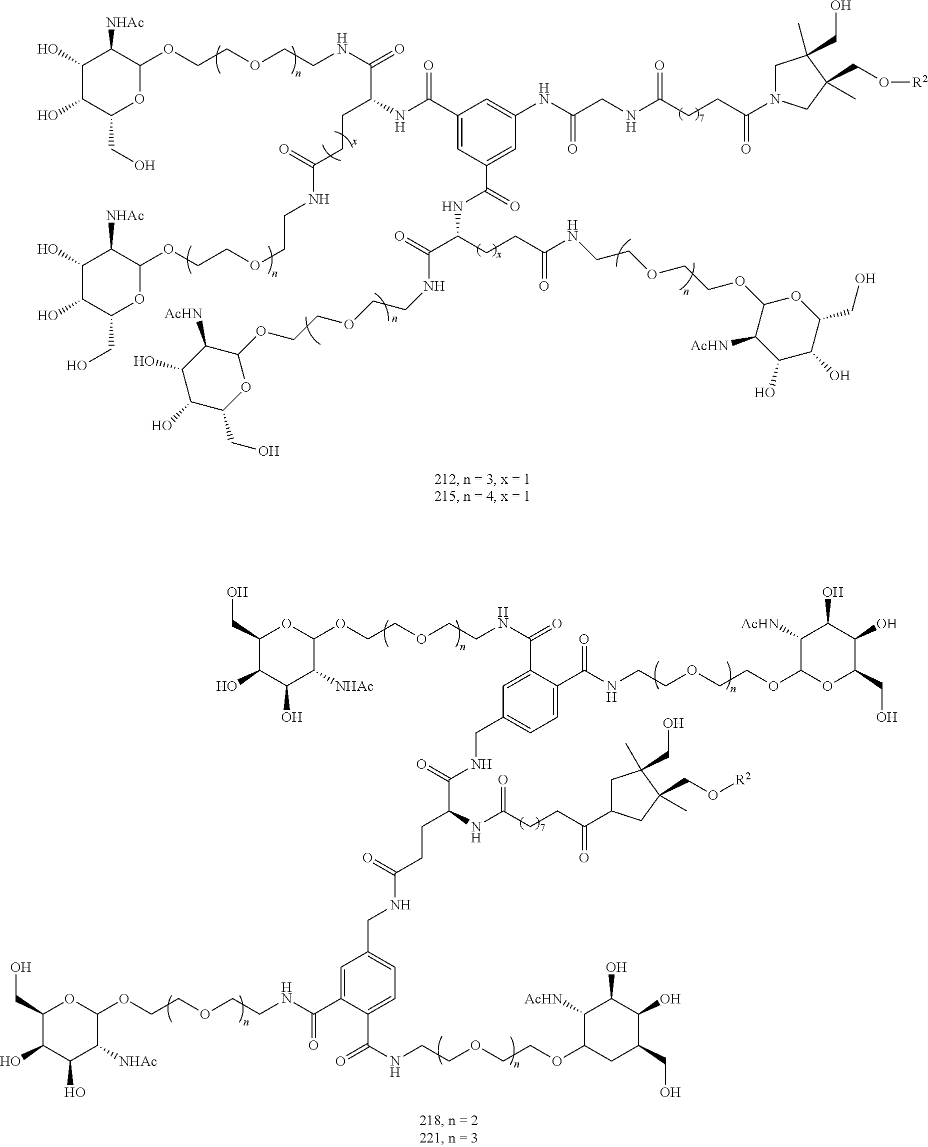 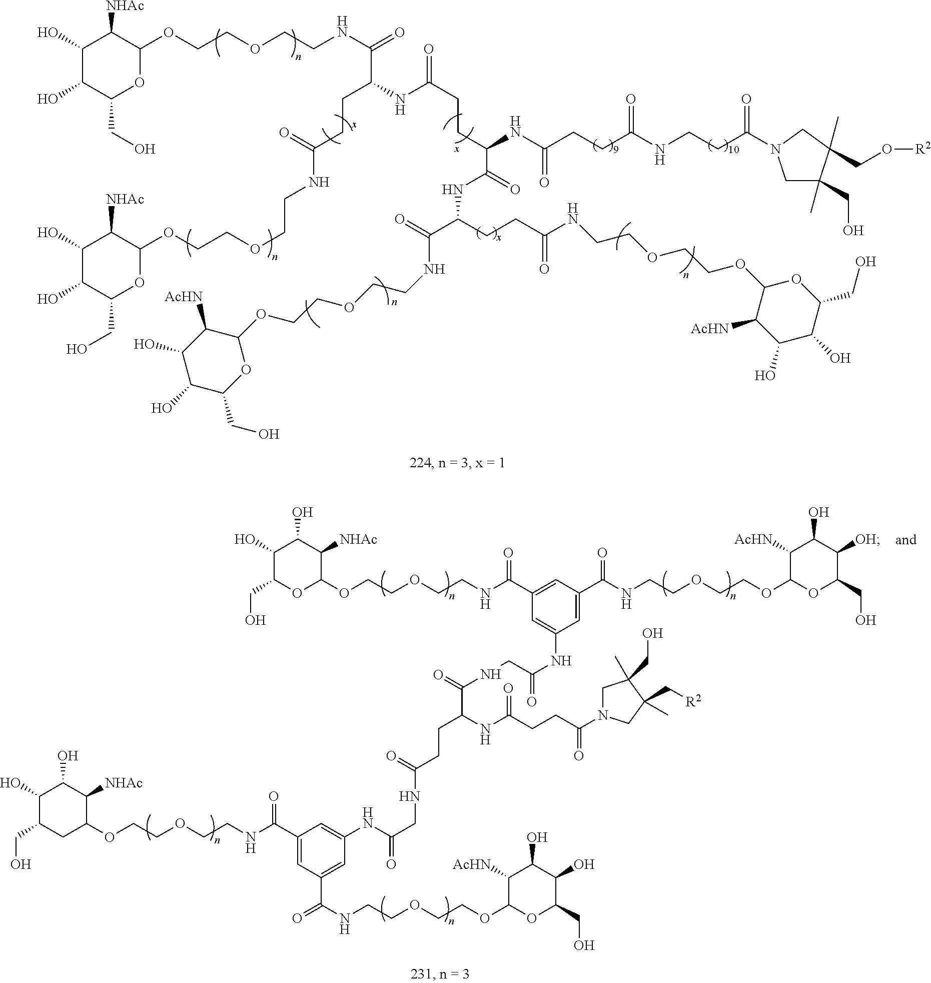
and pharmaceutically acceptable salts thereof.

7. The compound of claim 1, wherein the compound of formula (I) is: 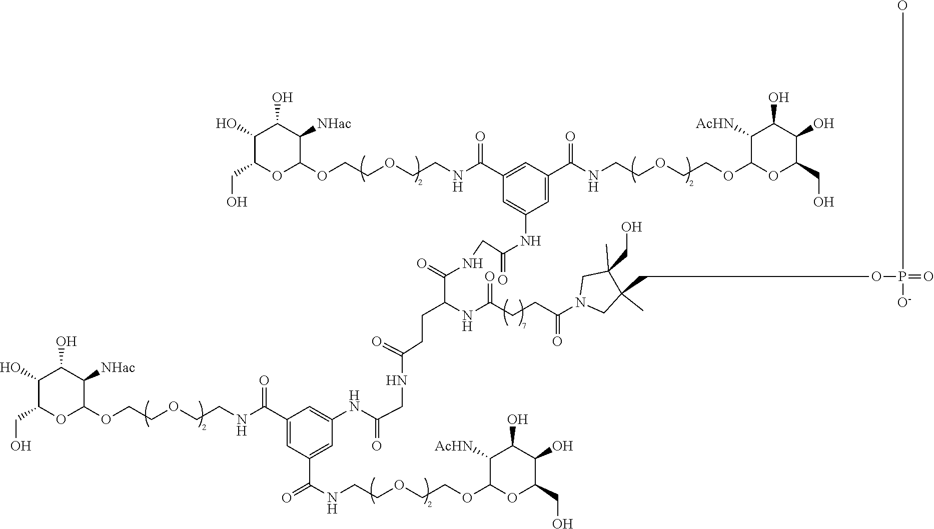
or a pharmaceutically acceptable salt thereof, wherein the siRNA depicted is selected from any one of siRNA 1-siRNA 119.

8. The compound of any one of claims 1-7, wherein the siRNA sequence comprises chemically modified nucleotides.

9. The compound of claim 8, wherein the siRNA comprises at least one 2′ Ome modification or a 2′F modification.

10. The compound of claim 9, wherein the siRNA comprises at least one 2′ Ome modification and at least one 2′F modification.

11. The compound of claim 9, wherein the siRNA comprises at least one 2′ Ome modification and at least one 2′F modification.

12. A method of treating liver fibrosis, comprising administering to a patient in need thereof an effective amount of the compound of any one of claims 1-11.

13. A method of treating non-alcoholic steatohepatitis (NASH), comprising administering to a patient in need thereof an effective amount of the compound of any one of claims 1-11.

14. A method of treating liver fibrosis associated with non-alcoholic steatohepatitis (NASH), comprising administering to a patient in need thereof an effective amount of the compound of any one of claims 1-11.

15. A method of treating alcoholic steatohepatitis (ASH), comprising administering to a patient in need thereof an effective amount of the compound of any one of claims 1-11.

16. A method of treating liver fibrosis associated with alcoholic steatohepatitis (ASH), comprising administering to a patient in need thereof an effective amount of the compound of any one of claims 1-11.

17. The use of an effective amount of the compound of any one of claims 1-11 to treat liver fibrosis.

18. The use of an effective amount of the compound of any one of claims 1-11 to treat non-alcoholic steatohepatitis (NASH) or alcoholic steatohepatitis (ASH).

19. The use of an effective amount of the compound of any one of claims 1-11 to treat liver fibrosis associated non-alcoholic steatohepatitis (NASH) or alcoholic steatohepatitis (ASH).

20. The method or use of any one of claims 12-19, wherein the compound of formula (I) is administered subcutaneously.

21. A double stranded siRNA molecule selected from the group consisting of siRNA 1-siRNA 119.

22. A composition comprising a double stranded siRNA molecule of claim 21.

23. An invention as described herein.
